SEQUENCE LISTING (250 sequences)

SEQ ID NO: 1 - DNA, 6 bp, Artificial Sequence
  Description of Artificial Sequence Synthetic oligonucleotide
aagctt

SEQ ID NO: 2 - DNA, 6 bp, Artificial Sequence
  Description of Artificial Sequence Synthetic oligonucleotide
aatatt

SEQ ID NO: 3 - DNA, 4 bp, Artificial Sequence
  Description of Artificial Sequence Synthetic oligonucleotide
aatt

SEQ ID NO: 4 - DNA, 6 bp, Artificial Sequence
  Description of Artificial Sequence Synthetic oligonucleotide
acatgt

SEQ ID NO: 5 - DNA, 6 bp, Artificial Sequence
  Description of Artificial Sequence Synthetic oligonucleotide
accggt

SEQ ID NO: 6 - DNA, 6 bp, Artificial Sequence
  Description of Artificial Sequence Synthetic oligonucleotide
acctgc

SEQ ID NO: 7 - DNA, 7 bp, Artificial Sequence
  Description of Artificial Sequence Synthetic oligonucleotide
accwggt

SEQ ID NO: 8 - DNA, 6 bp, Artificial Sequence
  Description of Artificial Sequence Synthetic oligonucleotide
acgcgt

SEQ ID NO: 9 - DNA, 5 bp, Artificial Sequence
  Description of Artificial Sequence Synthetic oligonucleotide
acggc

SEQ ID NO: 10 - DNA, 4 bp, Artificial Sequence
  Description of Artificial Sequence Synthetic oligonucleotide
acgt

SEQ ID NO: 11 - DNA, 5 bp, Artificial Sequence
  Description of Artificial Sequence Synthetic oligonucleotide
  modified_base (3)..(3): a, c, t, g, unknown or other
acngt

SEQ ID NO: 12 - DNA, 11 bp, Artificial Sequence
  Description of Artificial Sequence Synthetic oligonucleotide
  modified_base (3)..(6): a, c, t, g, unknown or other
acnnnngtayc

SEQ ID NO: 13 - DNA, 11 bp, Artificial Sequence
  Description of Artificial Sequence Synthetic oligonucleotide
  modified_base (3)..(7): a, c, t, g, unknown or other
acnnnnnctcc

SEQ ID NO: 14 - DNA, 6 bp, Artificial Sequence
  Description of Artificial Sequence Synthetic oligonucleotide
acrygt

SEQ ID NO: 15 - DNA, 6 bp, Artificial Sequence
  Description of Artificial Sequence Synthetic oligonucleotide
actagt

SEQ ID NO: 16 - DNA, 5 bp, Artificial Sequence
  Description of Artificial Sequence Synthetic oligonucleotide
actgg

SEQ ID NO: 17 - DNA, 6 bp, Artificial Sequence
  Description of Artificial Sequence Synthetic oligonucleotide
actggg

SEQ ID NO: 18 - DNA, 6 bp, Artificial Sequence
  Description of Artificial Sequence Synthetic oligonucleotide
agatct

SEQ ID NO: 19 - DNA, 6 bp, Artificial Sequence
  Description of Artificial Sequence Synthetic oligonucleotide
agcgct

SEQ ID NO: 20 - DNA, 4 bp, Artificial Sequence
  Description of Artificial Sequence Synthetic oligonucleotide
agct

SEQ ID NO: 21 - DNA, 6 bp, Artificial Sequence
  Description of Artificial Sequence Synthetic oligonucleotide
aggcct

SEQ ID NO: 22 - DNA, 6 bp, Artificial Sequence
  Description of Artificial Sequence Synthetic oligonucleotide
agtact

SEQ ID NO: 23 - DNA, 6 bp, Artificial Sequence
  Description of Artificial Sequence Synthetic oligonucleotide
atcgat

SEQ ID NO: 24 - DNA, 30 bp, Artificial Sequence
  Description of Artificial Sequence Synthetic oligonucleotide
atctatgtcgggtgcggagaaagaggtaat

SEQ ID NO: 25 - DNA, 6 bp, Artificial Sequence
  Description of Artificial Sequence Synthetic oligonucleotide
atgcat

SEQ ID NO: 26 - DNA, 6 bp, Artificial Sequence
  Description of Artificial Sequence Synthetic oligonucleotide
attaat

SEQ ID NO: 27 - DNA, 8 bp, Artificial Sequence
  Description of Artificial Sequence Synthetic oligonucleotide
atttaaat

SEQ ID NO: 28 - DNA, 12 bp, Artificial Sequence
  Description of Artificial Sequence Synthetic oligonucleotide
  modified_base (4)..(8): a, c, t, g, unknown or other
caannnnngtgg

SEQ ID NO: 29 - DNA, 6 bp, Artificial Sequence
  Description of Artificial Sequence Synthetic oligonucleotide
caattg

SEQ ID NO: 30 - DNA, 6 bp, Artificial Sequence
  Description of Artificial Sequence Synthetic oligonucleotide
cacgag

SEQ ID NO: 31 - DNA, 6 bp, Artificial Sequence
  Description of Artificial Sequence Synthetic oligonucleotide
cacgtc

SEQ ID NO: 32 - DNA, 6 bp, Artificial Sequence
  Description of Artificial Sequence Synthetic oligonucleotide
cacgtg

SEQ ID NO: 33 - DNA, 9 bp, Artificial Sequence
  Description of Artificial Sequence Synthetic oligonucleotide
  modified_base (4)..(6): a, c, t, g, unknown or other
cacnnngtg

SEQ ID NO: 34 - DNA, 10 bp, Artificial Sequence
  Description of Artificial Sequence Synthetic oligonucleotide
  modified_base (4)..(7): a, c, t, g, unknown or other
cacnnnngtg

SEQ ID NO: 35 - DNA, 6 bp, Artificial Sequence
  Description of Artificial Sequence Synthetic oligonucleotide
cagcag

SEQ ID NO: 36 - DNA, 6 bp, Artificial Sequence
  Description of Artificial Sequence Synthetic oligonucleotide
cagctg

SEQ ID NO: 37 - DNA, 9 bp, Artificial Sequence
  Description of Artificial Sequence Synthetic oligonucleotide
  modified_base (4)..(6): a, c, t, g, unknown or other
cagnnnctg

SEQ ID NO: 38 - DNA, 5 bp, Artificial Sequence
  Description of Artificial Sequence Synthetic oligonucleotide
cagtg

SEQ ID NO: 39 - DNA, 6 bp, Artificial Sequence
  Description of Artificial Sequence Synthetic oligonucleotide
catatg

SEQ ID NO: 40 - DNA, 4 bp, Artificial Sequence
  Description of Artificial Sequence Synthetic oligonucleotide
catg

SEQ ID NO: 41 - DNA, 4 bp, Artificial Sequence
  Description of Artificial Sequence Synthetic oligonucleotide
catg

SEQ ID NO: 42 - DNA, 10 bp, Artificial Sequence
  Description of Artificial Sequence Synthetic oligonucleotide
  modified_base (4)..(7): a, c, t, g, unknown or other
caynnnnrtg

SEQ ID NO: 43 - DNA, 2 bp, Artificial Sequence
  Description of Artificial Sequence Synthetic oligonucleotide
cc

SEQ ID NO: 44 - DNA, 15 bp, Artificial Sequence
  Description of Artificial Sequence Synthetic oligonucleotide
  modified_base (4)..(12): a, c, t, g, unknown or other
ccannnnnnnnntgg

SEQ ID NO: 45 - DNA, 12 bp, Artificial Sequence
  Description of Artificial Sequence Synthetic oligonucleotide
  modified_base (4)..(9): a, c, t, g, unknown or other
ccannnnnntgg

SEQ ID NO: 46 - DNA, 11 bp, Artificial Sequence
  Description of Artificial Sequence Synthetic oligonucleotide
  modified_base (4)..(8): a, c, t, g, unknown or other
ccannnnntgg

SEQ ID NO: 47 - DNA, 5 bp, Artificial Sequence
  Description of Artificial Sequence Synthetic oligonucleotide
ccatc

SEQ ID NO: 48 - DNA, 6 bp, Artificial Sequence
  Description of Artificial Sequence Synthetic oligonucleotide
ccatgg

SEQ ID NO: 49 - DNA, 6 bp, Artificial Sequence
  Description of Artificial Sequence Synthetic oligonucleotide
cccagc

SEQ ID NO: 50 - DNA, 5 bp, Artificial Sequence
  Description of Artificial Sequence Synthetic oligonucleotide
cccgc

SEQ ID NO: 51 - DNA, 6 bp, Artificial Sequence
  Description of Artificial Sequence Synthetic oligonucleotide
cccggg

SEQ ID NO: 52 - DNA, 6 bp, Artificial Sequence
  Description of Artificial Sequence Synthetic oligonucleotide
cccggg

SEQ ID NO: 53 - DNA, 3 bp, Artificial Sequence
  Description of Artificial Sequence Synthetic oligonucleotide
ccd

SEQ ID NO: 54 - DNA, 4 bp, Artificial Sequence
  Description of Artificial Sequence Synthetic oligonucleotide
ccdg

SEQ ID NO: 55 - DNA, 4 bp, Artificial Sequence
  Description of Artificial Sequence Synthetic oligonucleotide
ccgc

SEQ ID NO: 56 - DNA, 6 bp, Artificial Sequence
  Description of Artificial Sequence Synthetic oligonucleotide
ccgcgg

SEQ ID NO: 57 - DNA, 6 bp, Artificial Sequence
  Description of Artificial Sequence Synthetic oligonucleotide
ccgctc

SEQ ID NO: 58 - DNA, 4 bp, Artificial Sequence
  Description of Artificial Sequence Synthetic oligonucleotide
ccgg

SEQ ID NO: 59 - DNA, 5 bp, Artificial Sequence
  Description of Artificial Sequence Synthetic oligonucleotide
  modified_base (3)..(3): a, c, t, g, unknown or other
ccngg

SEQ ID NO: 60 - DNA, 5 bp, Artificial Sequence
  Description of Artificial Sequence Synthetic oligonucleotide
  modified_base (3)..(3): a, c, t, g, unknown or other
ccngg

SEQ ID NO: 61 - DNA, 6 bp, Artificial Sequence
  Description of Artificial Sequence Synthetic oligonucleotide
  modified_base (3)..(4): a, c, t, g, unknown or other
ccnngg

SEQ ID NO: 62 - DNA, 11 bp, Artificial Sequence
  Description of Artificial Sequence Synthetic oligonucleotide
  modified_base (3)..(9): a, c, t, g, unknown or other
ccnnnnnnngg

SEQ ID NO: 63 - DNA, 6 bp, Artificial Sequence
  Description of Artificial Sequence Synthetic oligonucleotide
ccrygg

SEQ ID NO: 64 - DNA, 5 bp, Artificial Sequence
  Description of Artificial Sequence Synthetic oligonucleotide
ccsgg

SEQ ID NO: 65 - DNA, 6 bp, Artificial Sequence
  Description of Artificial Sequence Synthetic oligonucleotide
cctagg

SEQ ID NO: 66 - DNA, 4 bp, Artificial Sequence
  Description of Artificial Sequence Synthetic oligonucleotide
cctc

SEQ ID NO: 67 - DNA, 7 bp, Artificial Sequence
  Description of Artificial Sequence Synthetic oligonucleotide
cctcagc

SEQ ID NO: 68 - DNA, 7 bp, Artificial Sequence
  Description of Artificial Sequence Synthetic oligonucleotide
cctcagc

SEQ ID NO: 69 - DNA, 8 bp, Artificial Sequence
  Description of Artificial Sequence Synthetic oligonucleotide
cctgcagg

SEQ ID NO: 70 - DNA, 7 bp, Artificial Sequence
  Description of Artificial Sequence Synthetic oligonucleotide
  modified_base (4)..(4): a, c, t, g, unknown or other
cctnagc

SEQ ID NO: 71 - DNA, 7 bp, Artificial Sequence
  Description of Artificial Sequence Synthetic oligonucleotide
  modified_base (4)..(4): a, c, t, g, unknown or other
cctnagg

SEQ ID NO: 72 - DNA, 11 bp, Artificial Sequence
  Description of Artificial Sequence Synthetic oligonucleotide
  modified_base (4)..(8): a, c, t, g, unknown or other
cctnnnnnagg

SEQ ID NO: 73 - DNA, 5 bp, Artificial Sequence
  Description of Artificial Sequence Synthetic oligonucleotide
ccttc

SEQ ID NO: 74 - DNA, 5 bp, Artificial Sequence
  Description of Artificial Sequence Synthetic oligonucleotide
ccwgg

SEQ ID NO: 75 - DNA, 5 bp, Artificial Sequence
  Description of Artificial Sequence Synthetic oligonucleotide
ccwgg

SEQ ID NO: 76 - DNA, 6 bp, Artificial Sequence
  Description of Artificial Sequence Synthetic oligonucleotide
ccwwgg

SEQ ID NO: 77 - DNA, 12 bp, Artificial Sequence
  Description of Artificial Sequence Synthetic oligonucleotide
  modified_base (4)..(9): a, c, t, g, unknown or other
cgannnnnntgc

SEQ ID NO: 78 - DNA, 6 bp, Artificial Sequence
  Description of Artificial Sequence Synthetic oligonucleotide
cgatcg

SEQ ID NO: 79 - DNA, 4 bp, Artificial Sequence
  Description of Artificial Sequence Synthetic oligonucleotide
cgcg

SEQ ID NO: 80 - DNA, 6 bp, Artificial Sequence
  Description of Artificial Sequence Synthetic oligonucleotide
cggccg

SEQ ID NO: 81 - DNA, 7 bp, Artificial Sequence
  Description of Artificial Sequence Synthetic oligonucleotide
cggwccg

SEQ ID NO: 82 - DNA, 6 bp, Artificial Sequence
  Description of Artificial Sequence Synthetic oligonucleotide
cgrycg

SEQ ID NO: 83 - DNA, 6 bp, Artificial Sequence
  Description of Artificial Sequence Synthetic oligonucleotide
cgtacg

SEQ ID NO: 84 - DNA, 6 bp, Artificial Sequence
  Description of Artificial Sequence Synthetic oligonucleotide
cgtctc

SEQ ID NO: 85 - DNA, 5 bp, Artificial Sequence
  Description of Artificial Sequence Synthetic oligonucleotide
cgwcg

SEQ ID NO: 86 - DNA, 6 bp, Artificial Sequence
  Description of Artificial Sequence Synthetic oligonucleotide
cmgckg

SEQ ID NO: 87 - DNA, 22 bp, Artificial Sequence
  Description of Artificial Sequence Synthetic oligonucleotide
  modified_base (2)..(21): a, c, t, g, unknown or other
cnnnnnnnnnnnnnnnnnnnng

SEQ ID NO: 88 - DNA, 4 bp, Artificial Sequence
  Description of Artificial Sequence Synthetic oligonucleotide
  modified_base (2)..(3): a, c, t, g, unknown or other
cnnr

SEQ ID NO: 89 - DNA, 8 bp, Artificial Sequence
  Description of Artificial Sequence Synthetic oligonucleotide
crccggyg

SEQ ID NO: 90 - DNA, 4 bp, Artificial Sequence
  Description of Artificial Sequence Synthetic oligonucleotide
ctag

SEQ ID NO: 91 - DNA, 5 bp, Artificial Sequence
  Description of Artificial Sequence Synthetic oligonucleotide
ctcag

SEQ ID NO: 92 - DNA, 6 bp, Artificial Sequence
  Description of Artificial Sequence Synthetic oligonucleotide
ctcgag

SEQ ID NO: 93 - DNA, 6 bp, Artificial Sequence
  Description of Artificial Sequence Synthetic oligonucleotide
ctcttc

SEQ ID NO: 94 - DNA, 6 bp, Artificial Sequence
  Description of Artificial Sequence Synthetic oligonucleotide
ctgaag

SEQ ID NO: 95 - DNA, 6 bp, Artificial Sequence
  Description of Artificial Sequence Synthetic oligonucleotide
ctgcag

SEQ ID NO: 96 - DNA, 6 bp, Artificial Sequence
  Description of Artificial Sequence Synthetic oligonucleotide
ctggag

SEQ ID NO: 97 - DNA, 5 bp, Artificial Sequence
  Description of Artificial Sequence Synthetic oligonucleotide
  modified_base (3)..(3): a, c, t, g, unknown or other
ctnag

SEQ ID NO: 98 - DNA, 6 bp, Artificial Sequence
  Description of Artificial Sequence Synthetic oligonucleotide
ctryag

SEQ ID NO: 99 - DNA, 6 bp, Artificial Sequence
  Description of Artificial Sequence Synthetic oligonucleotide
cttaag

SEQ ID NO: 100 - DNA, 6 bp, Artificial Sequence
  Description of Artificial Sequence Synthetic oligonucleotide
cttgag

SEQ ID NO: 101 - DNA, 6 bp, Artificial Sequence
  Description of Artificial Sequence Synthetic oligonucleotide
ctyrag

SEQ ID NO: 102 - DNA, 6 bp, Artificial Sequence
  Description of Artificial Sequence Synthetic oligonucleotide
cycgrg

SEQ ID NO: 103 - DNA, 5 bp, Artificial Sequence
  Description of Artificial Sequence Synthetic oligonucleotide
gaaga

SEQ ID NO: 104 - DNA, 6 bp, Artificial Sequence
  Description of Artificial Sequence Synthetic oligonucleotide
gaagac

SEQ ID NO: 105 - DNA, 10 bp, Artificial Sequence
  Description of Artificial Sequence Synthetic oligonucleotide
  modified_base (4)..(7): a, c, t, g, unknown or other
gaannnnttc

SEQ ID NO: 106 - DNA, 6 bp, Artificial Sequence
  Description of Artificial Sequence Synthetic oligonucleotide
gaatgc

SEQ ID NO: 107 - DNA, 6 bp, Artificial Sequence
  Description of Artificial Sequence Synthetic oligonucleotide
gaattc

SEQ ID NO: 108 - DNA, 5 bp, Artificial Sequence
  Description of Artificial Sequence Synthetic oligonucleotide
gacgc

SEQ ID NO: 109 - DNA, 6 bp, Artificial Sequence
  Description of Artificial Sequence Synthetic oligonucleotide
gacgtc

SEQ ID NO: 110 - DNA, 6 bp, Artificial Sequence
  Description of Artificial Sequence Synthetic oligonucleotide
gacgtc

SEQ ID NO: 111 - DNA, 9 bp, Artificial Sequence
  Description of Artificial Sequence Synthetic oligonucleotide
  modified_base (4)..(6): a, c, t, g, unknown or other
gacnnngtc

SEQ ID NO: 112 - DNA, 10 bp, Artificial Sequence
  Description of Artificial Sequence Synthetic oligonucleotide
  modified_base (4)..(7): a, c, t, g, unknown or other
gacnnnngtc

SEQ ID NO: 113 - DNA, 11 bp, Artificial Sequence
  Description of Artificial Sequence Synthetic oligonucleotide
  modified_base (4)..(8): a, c, t, g, unknown or other
gacnnnnngtc

SEQ ID NO: 114 - DNA, 12 bp, Artificial Sequence
  Description of Artificial Sequence Synthetic oligonucleotide
  modified_base (4)..(9): a, c, t, g, unknown or other
gacnnnnnngtc

SEQ ID NO: 115 - DNA, 6 bp, Artificial Sequence
  Description of Artificial Sequence Synthetic oligonucleotide
gagctc

SEQ ID NO: 116 - DNA, 6 bp, Artificial Sequence
  Description of Artificial Sequence Synthetic oligonucleotide
gagctc

SEQ ID NO: 117 - DNA, 6 bp, Artificial Sequence
  Description of Artificial Sequence Synthetic oligonucleotide
gaggag

SEQ ID NO: 118 - DNA, 5 bp, Artificial Sequence
  Description of Artificial Sequence Synthetic oligonucleotide
gagtc

SEQ ID NO: 119 - DNA, 5 bp, Artificial Sequence
  Description of Artificial Sequence Synthetic oligonucleotide
gagtc

SEQ ID NO: 120 - DNA, 5 bp, Artificial Sequence
  Description of Artificial Sequence Synthetic oligonucleotide
gagtc

SEQ ID NO: 121 - DNA, 5 bp, Artificial Sequence
  Description of Artificial Sequence Synthetic oligonucleotide
  modified_base (3)..(3): a, c, t, g, unknown or other
gantc

SEQ ID NO: 122 - DNA, 6 bp, Artificial Sequence
  Description of Artificial Sequence Synthetic oligonucleotide
gatatc

SEQ ID NO: 123 - DNA, 4 bp, Artificial Sequence
  Description of Artificial Sequence Synthetic oligonucleotide
gatc

SEQ ID NO: 124 - DNA, 4 bp, Artificial Sequence
  Description of Artificial Sequence Synthetic oligonucleotide
gatc

SEQ ID NO: 125 - DNA, 10 bp, Artificial Sequence
  Description of Artificial Sequence Synthetic oligonucleotide
  modified_base (4)..(7): a, c, t, g, unknown or other
gatnnnnatc

SEQ ID NO: 126 - DNA, 5 bp, Artificial Sequence
  Description of Artificial Sequence Synthetic oligonucleotide
gawtc

SEQ ID NO: 127 - DNA, 6 bp, Artificial Sequence
  Description of Artificial Sequence Synthetic oligonucleotide
gcaatg

SEQ ID NO: 128 - DNA, 5 bp, Artificial Sequence
  Description of Artificial Sequence Synthetic oligonucleotide
gcagc

SEQ ID NO: 129 - DNA, 6 bp, Artificial Sequence
  Description of Artificial Sequence Synthetic oligonucleotide
gcagtg

SEQ ID NO: 130 - DNA, 11 bp, Artificial Sequence
  Description of Artificial Sequence Synthetic oligonucleotide
  modified_base (4)..(8): a, c, t, g, unknown or other
gcannnnntgc

SEQ ID NO: 131 - DNA, 5 bp, Artificial Sequence
  Description of Artificial Sequence Synthetic oligonucleotide
gcatc

SEQ ID NO: 132 - DNA, 6 bp, Artificial Sequence
  Description of Artificial Sequence Synthetic oligonucleotide
gcatgc

SEQ ID NO: 133 - DNA, 8 bp, Artificial Sequence
  Description of Artificial Sequence Synthetic oligonucleotide
gcccgggc

SEQ ID NO: 134 - DNA, 6 bp, Artificial Sequence
  Description of Artificial Sequence Synthetic oligonucleotide
gccgag

SEQ ID NO: 135 - DNA, 6 bp, Artificial Sequence
  Description of Artificial Sequence Synthetic oligonucleotide
gccggc

SEQ ID NO: 136 - DNA, 6 bp, Artificial Sequence
  Description of Artificial Sequence Synthetic oligonucleotide
gccggc

SEQ ID NO: 137 - DNA, 11 bp, Artificial Sequence
  Description of Artificial Sequence Synthetic oligonucleotide
  modified_base (4)..(8): a, c, t, g, unknown or other
gccnnnnnggc

SEQ ID NO: 138 - DNA, 8 bp, Artificial Sequence
  Description of Artificial Sequence Synthetic oligonucleotide
gcgatcgc

SEQ ID NO: 139 - DNA, 6 bp, Artificial Sequence
  Description of Artificial Sequence Synthetic oligonucleotide
gcgatg

SEQ ID NO: 140 - DNA, 4 bp, Artificial Sequence
  Description of Artificial Sequence Synthetic oligonucleotide
gcgc

SEQ ID NO: 141 - DNA, 4 bp, Artificial Sequence
  Description of Artificial Sequence Synthetic oligonucleotide
gcgc

SEQ ID NO: 142 - DNA, 6 bp, Artificial Sequence
  Description of Artificial Sequence Synthetic oligonucleotide
gcgcgc

SEQ ID NO: 143 - DNA, 8 bp, Artificial Sequence
  Description of Artificial Sequence Synthetic oligonucleotide
gcggccgc

SEQ ID NO: 144 - DNA, 5 bp, Artificial Sequence
  Description of Artificial Sequence Synthetic oligonucleotide
  modified_base (3)..(3): a, c, t, g, unknown or other
gcngc

SEQ ID NO: 145 - DNA, 6 bp, Artificial Sequence
  Description of Artificial Sequence Synthetic oligonucleotide
  modified_base (3)..(4): a, c, t, g, unknown or other
gcnngc

SEQ ID NO: 146 - DNA, 11 bp, Artificial Sequence
  Description of Artificial Sequence Synthetic oligonucleotide
  modified_base (3)..(9): a, c, t, g, unknown or other
gcnnnnnnngc

SEQ ID NO: 147 - DNA, 6 bp, Artificial Sequence
  Description of Artificial Sequence Synthetic oligonucleotide
gctagc

SEQ ID NO: 148 - DNA, 6 bp, Artificial Sequence
  Description of Artificial Sequence Synthetic oligonucleotide
gctagc

SEQ ID NO: 149 - DNA, 7 bp, Artificial Sequence
  Description of Artificial Sequence Synthetic oligonucleotide
gctcttc

SEQ ID NO: 150 - DNA, 7 bp, Artificial Sequence
  Description of Artificial Sequence Synthetic oligonucleotide
gctcttc

SEQ ID NO: 151 - DNA, 7 bp, Artificial Sequence
  Description of Artificial Sequence Synthetic oligonucleotide
  modified_base (4)..(4): a, c, t, g, unknown or other
gctnagc

SEQ ID NO: 152 - DNA, 5 bp, Artificial Sequence
  Description of Artificial Sequence Synthetic oligonucleotide
gcwgc

SEQ ID NO: 153 - DNA, 6 bp, Artificial Sequence
  Description of Artificial Sequence Synthetic oligonucleotide
gdgchc

SEQ ID NO: 154 - DNA, 5 bp, Artificial Sequence
  Description of Artificial Sequence Synthetic oligonucleotide
ggatc

SEQ ID NO: 155 - DNA, 5 bp, Artificial Sequence
  Description of Artificial Sequence Synthetic oligonucleotide
ggatc

SEQ ID NO: 156 - DNA, 6 bp, Artificial Sequence
  Description of Artificial Sequence Synthetic oligonucleotide
ggatcc

SEQ ID NO: 157 - DNA, 5 bp, Artificial Sequence
  Description of Artificial Sequence Synthetic oligonucleotide
ggatg

SEQ ID NO: 158 - DNA, 5 bp, Artificial Sequence
  Description of Artificial Sequence Synthetic oligonucleotide
ggatg

SEQ ID NO: 159 - DNA, 4 bp, Artificial Sequence
  Description of Artificial Sequence Synthetic oligonucleotide
ggcc

SEQ ID NO: 160 - DNA, 8 bp, Artificial Sequence
  Description of Artificial Sequence Synthetic oligonucleotide
ggccggcc

SEQ ID NO: 161 - DNA, 13 bp, Artificial Sequence
  Description of Artificial Sequence Synthetic oligonucleotide
  modified_base (5)..(9): a, c, t, g, unknown or other
ggccnnnnnggcc

SEQ ID NO: 162 - DNA, 6 bp, Artificial Sequence
  Description of Artificial Sequence Synthetic oligonucleotide
ggcgcc

SEQ ID NO: 163 - DNA, 6 bp, Artificial Sequence
  Description of Artificial Sequence Synthetic oligonucleotide
ggcgcc

SEQ ID NO: 164 - DNA, 6 bp, Artificial Sequence
  Description of Artificial Sequence Synthetic oligonucleotide
ggcgcc

SEQ ID NO: 165 - DNA, 6 bp, Artificial Sequence
  Description of Artificial Sequence Synthetic oligonucleotide
ggcgcc

SEQ ID NO: 166 - DNA, 8 bp, Artificial Sequence
  Description of Artificial Sequence Synthetic oligonucleotide
ggcgcgcc

SEQ ID NO: 167 - DNA, 6 bp, Artificial Sequence
  Description of Artificial Sequence Synthetic oligonucleotide
ggcgga

SEQ ID NO: 168 - DNA, 5 bp, Artificial Sequence
  Description of Artificial Sequence Synthetic oligonucleotide
gggac

SEQ ID NO: 169 - DNA, 6 bp, Artificial Sequence
  Description of Artificial Sequence Synthetic oligonucleotide
gggccc

SEQ ID NO: 170 - DNA, 6 bp, Artificial Sequence
  Description of Artificial Sequence Synthetic oligonucleotide
gggccc

SEQ ID NO: 171 - DNA, 5 bp, Artificial Sequence
  Description of Artificial Sequence Synthetic oligonucleotide
  modified_base (3)..(3): a, c, t, g, unknown or other
ggncc

SEQ ID NO: 172 - DNA, 6 bp, Artificial Sequence
  Description of Artificial Sequence Synthetic oligonucleotide
  modified_base (3)..(4): a, c, t, g, unknown or other
ggnncc

SEQ ID NO: 173 - DNA, 6 bp, Artificial Sequence
  Description of Artificial Sequence Synthetic oligonucleotide
ggtacc

SEQ ID NO: 174 - DNA, 6 bp, Artificial Sequence
  Description of Artificial Sequence Synthetic oligonucleotide
ggtacc

SEQ ID NO: 175 - DNA, 6 bp, Artificial Sequence
  Description of Artificial Sequence Synthetic oligonucleotide
ggtctc

SEQ ID NO: 176 - DNA, 5 bp, Artificial Sequence
  Description of Artificial Sequence Synthetic oligonucleotide
ggtga

SEQ ID NO: 177 - DNA, 7 bp, Artificial Sequence
  Description of Artificial Sequence Synthetic oligonucleotide
  modified_base (4)..(4): a, c, t, g, unknown or other
ggtnacc

SEQ ID NO: 178 - DNA, 5 bp, Artificial Sequence
  Description of Artificial Sequence Synthetic oligonucleotide
ggwcc

SEQ ID NO: 179 - DNA, 6 bp, Artificial Sequence
  Description of Artificial Sequence Synthetic oligonucleotide
ggyrcc

SEQ ID NO: 180 - DNA, 6 bp, Artificial Sequence
  Description of Artificial Sequence Synthetic oligonucleotide
gkgcmc

SEQ ID NO: 181 - DNA, 6 bp, Artificial Sequence
  Description of Artificial Sequence Synthetic oligonucleotide
grcgyc

SEQ ID NO: 182 - DNA, 6 bp, Artificial Sequence
  Description of Artificial Sequence Synthetic oligonucleotide
grgcyc

SEQ ID NO: 183 - DNA, 4 bp, Artificial Sequence
  Description of Artificial Sequence Synthetic oligonucleotide
gtac

SEQ ID NO: 184 - DNA, 4 bp, Artificial Sequence
  Description of Artificial Sequence Synthetic oligonucleotide
gtac

SEQ ID NO: 185 - DNA, 6 bp, Artificial Sequence
  Description of Artificial Sequence Synthetic oligonucleotide
gtatac

SEQ ID NO: 186 - DNA, 6 bp, Artificial Sequence
  Description of Artificial Sequence Synthetic oligonucleotide
gtatcc

SEQ ID NO: 187 - DNA, 6 bp, Artificial Sequence
  Description of Artificial Sequence Synthetic oligonucleotide
gtcgac

SEQ ID NO: 188 - DNA, 5 bp, Artificial Sequence
  Description of Artificial Sequence Synthetic oligonucleotide
gtctc

SEQ ID NO: 189 - DNA, 5 bp, Artificial Sequence
  Description of Artificial Sequence Synthetic oligonucleotide
gtctc

SEQ ID NO: 190 - DNA, 6 bp, Artificial Sequence
  Description of Artificial Sequence Synthetic oligonucleotide
gtgcac

SEQ ID NO: 191 - DNA, 6 bp, Artificial Sequence
  Description of Artificial Sequence Synthetic oligonucleotide
gtgcag

SEQ ID NO: 192 - DNA, 6 bp, Artificial Sequence
  Description of Artificial Sequence Synthetic oligonucleotide
gtmkac

SEQ ID NO: 193 - DNA, 6 bp, Artificial Sequence
  Description of Artificial Sequence Synthetic oligonucleotide
  modified_base (3)..(4): a, c, t, g, unknown or other
gtnnac

SEQ ID NO: 194 - DNA, 5 bp, Artificial Sequence
  Description of Artificial Sequence Synthetic oligonucleotide
gtsac

SEQ ID NO: 195 - DNA, 6 bp, Artificial Sequence
  Description of Artificial Sequence Synthetic oligonucleotide
gttaac

SEQ ID NO: 196 - DNA, 8 bp, Artificial Sequence
  Description of Artificial Sequence Synthetic oligonucleotide
gtttaaac

SEQ ID NO: 197 - DNA, 6 bp, Artificial Sequence
  Description of Artificial Sequence Synthetic oligonucleotide
gtyrac

SEQ ID NO: 198 - DNA, 6 bp, Artificial Sequence
  Description of Artificial Sequence Synthetic oligonucleotide
gwgcwc

SEQ ID NO: 199 - DNA, 9 bp, Artificial Sequence
  Description of Artificial Sequence Synthetic oligonucleotide
  modified_base (1)..(2): a, c, t, g, unknown or other
  modified_base (8)..(9): a, c, t, g, unknown or other
nncastgnn

SEQ ID NO: 200 - DNA, 6 bp, Artificial Sequence
  Description of Artificial Sequence Synthetic oligonucleotide
raatty

SEQ ID NO: 201 - DNA, 6 bp, Artificial Sequence
  Description of Artificial Sequence Synthetic oligonucleotide
rcatgy

SEQ ID NO: 202 - DNA, 6 bp, Artificial Sequence
  Description of Artificial Sequence Synthetic oligonucleotide
rccggy

SEQ ID NO: 203 - DNA, 6 bp, Artificial Sequence
  Description of Artificial Sequence Synthetic oligonucleotide
rgatcy

SEQ ID NO: 204 - DNA, 6 bp, Artificial Sequence
  Description of Artificial Sequence Synthetic oligonucleotide
rgcgcy

SEQ ID NO: 205 - DNA, 4 bp, Artificial Sequence
  Description of Artificial Sequence Synthetic oligonucleotide
rgcy

SEQ ID NO: 206 - DNA, 7 bp, Artificial Sequence
  Description of Artificial Sequence Synthetic oligonucleotide
  modified_base (4)..(4): a, c, t, g, unknown or other
rggnccy

SEQ ID NO: 207 - DNA, 7 bp, Artificial Sequence
  Description of Artificial Sequence Synthetic oligonucleotide
rggwccy

SEQ ID NO: 208 - DNA, 27 bp, Artificial Sequence
  Description of Artificial Sequence Synthetic oligonucleotide
taactataacggtcctaaggtagcgaa

SEQ ID NO: 209 - DNA, 6 bp, Artificial Sequence
  Description of Artificial Sequence Synthetic oligonucleotide
tacgta

SEQ ID NO: 210 - DNA, 18 bp, Artificial Sequence
  Description of Artificial Sequence Synthetic oligonucleotide
tagggataacagggtaat

SEQ ID NO: 211 - DNA, 6 bp, Artificial Sequence
  Description of Artificial Sequence Synthetic oligonucleotide
tcatga

SEQ ID NO: 212 - DNA, 6 bp, Artificial Sequence
  Description of Artificial Sequence Synthetic oligonucleotide
tccgga

SEQ ID NO: 213 - DNA, 6 bp, Artificial Sequence
  Description of Artificial Sequence Synthetic oligonucleotide
tccrac

SEQ ID NO: 214 - DNA, 4 bp, Artificial Sequence
  Description of Artificial Sequence Synthetic oligonucleotide
tcga

SEQ ID NO: 215 - DNA, 6 bp, Artificial Sequence
  Description of Artificial Sequence Synthetic oligonucleotide
tcgcga

SEQ ID NO: 216 - DNA, 5 bp, Artificial Sequence
  Description of Artificial Sequence Synthetic oligonucleotide
  modified_base (3)..(3): a, c, t, g, unknown or other
tcnga

SEQ ID NO: 217 - DNA, 6 bp, Artificial Sequence
  Description of Artificial Sequence Synthetic oligonucleotide
  modified_base (3)..(4): a, c, t, g, unknown or other
tcnnga

SEQ ID NO: 218 - DNA, 6 bp, Artificial Sequence
  Description of Artificial Sequence Synthetic oligonucleotide
tctaga

SEQ ID NO: 219 - DNA, 6 bp, Artificial Sequence
  Description of Artificial Sequence Synthetic oligonucleotide
tgatca

SEQ ID NO: 220 - DNA, 4 bp, Artificial Sequence
  Description of Artificial Sequence Synthetic oligonucleotide
tgca

SEQ ID NO: 221 - DNA, 6 bp, Artificial Sequence
  Description of Artificial Sequence Synthetic oligonucleotide
tgcgca

SEQ ID NO: 222 - DNA, 30 bp, Artificial Sequence
  Description of Artificial Sequence Synthetic oligonucleotide
tggcaaacagctattatgggtattatgggt

SEQ ID NO: 223 - DNA, 6 bp, Artificial Sequence
  Description of Artificial Sequence Synthetic oligonucleotide
tggcca

SEQ ID NO: 224 - DNA, 6 bp, Artificial Sequence
  Description of Artificial Sequence Synthetic oligonucleotide
tgtaca

SEQ ID NO: 225 - DNA, 4 bp, Artificial Sequence
  Description of Artificial Sequence Synthetic oligonucleotide
ttaa

SEQ ID NO: 226 - DNA, 8 bp, Artificial Sequence
  Description of Artificial Sequence Synthetic oligonucleotide
ttaattaa

SEQ ID NO: 227 - DNA, 6 bp, Artificial Sequence
  Description of Artificial Sequence Synthetic oligonucleotide
ttataa

SEQ ID NO: 228 - DNA, 6 bp, Artificial Sequence
  Description of Artificial Sequence Synthetic oligonucleotide
ttcgaa

SEQ ID NO: 229 - DNA, 6 bp, Artificial Sequence
  Description of Artificial Sequence Synthetic oligonucleotide
tttaaa

SEQ ID NO: 230 - DNA, 8 bp, Artificial Sequence
  Description of Artificial Sequence Synthetic oligonucleotide
vctcgagb

SEQ ID NO: 231 - DNA, 6 bp, Artificial Sequence
  Description of Artificial Sequence Synthetic oligonucleotide
wccggw

SEQ ID NO: 232 - DNA, 6 bp, Artificial Sequence
  Description of Artificial Sequence Synthetic oligonucleotide
yacgtr

SEQ ID NO: 233 - DNA, 6 bp, Artificial Sequence
  Description of Artificial Sequence Synthetic oligonucleotide
yggccr

SEQ ID NO: 234 - DNA, 14 bp, Artificial Sequence
  Description of Artificial Sequence Synthetic oligonucleotide
gcttcgatcgaagc

SEQ ID NO: 235 - DNA, 430 bp, Artificial Sequence
  Description of Artificial Sequence Synthetic polynucleotide
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatta

SEQ ID NO: 236 - DNA, 381 bp, Artificial Sequence
  Description of Artificial Sequence Synthetic polynucleotide
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcgg

SEQ ID NO: 237 - DNA, 329 bp, Artificial Sequence
  Description of Artificial Sequence Synthetic polynucleotide
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggtttaacactc

SEQ ID NO: 238 - DNA, 329 bp, Artificial Sequence
  Description of Artificial Sequence Synthetic polynucleotide
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggtttaacactc

SEQ ID NO: 239 - DNA, 56 bp, Artificial Sequence
  Description of Artificial Sequence Synthetic oligonucleotide
tatcagcacacaattgcccattatacgcgcgtataatggactattgtgtgctgata

SEQ ID NO: 240 - DNA, 6 bp, Artificial Sequence
  Description of Artificial Sequence Synthetic oligonucleotide
gtaatg

SEQ ID NO: 241 - DNA, 6 bp, Artificial Sequence
  Description of Artificial Sequence Synthetic oligonucleotide
gtatac

SEQ ID NO: 242 - DNA, 6 bp, Artificial Sequence
  Description of Artificial Sequence Synthetic oligonucleotide
catatg

SEQ ID NO: 243 - DNA, 39 bp, Artificial Sequence
  Description of Artificial Sequence Synthetic oligonucleotide
ccattctgttccgcatgattcctctgcggaacagaatgg

SEQ ID NO: 244 - DNA, 199 bp, Artificial Sequence
  Description of Artificial Sequence Synthetic polynucleotide
ggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 245 - DNA, 402 bp, Artificial Sequence
  Description of Artificial Sequence Synthetic polynucleotide
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 246 - DNA, 212 bp, Artificial Sequence
  Description of Artificial Sequence Synthetic polynucleotide
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccag

SEQ ID NO: 247 - DNA, 180 bp, Artificial Sequence
  Description of Artificial Sequence Synthetic polynucleotide
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtg

SEQ ID NO: 248 - DNA, 103 bp, Artificial Sequence
  Description of Artificial Sequence Synthetic polynucleotide
caggctttcactttctcgccaacttacaaggcctttctgtgtaaacaatacctgaacctttaccccgttgcccggcaacggccaggtctgtgccaagtgtttg

SEQ ID NO: 249 - DNA, 305 bp, Artificial Sequence
  Description of Artificial Sequence Synthetic polynucleotide
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 250 - DNA, 11 bp, Artificial Sequence
  Description of Artificial Sequence Synthetic oligonucleotide
  modified_base (1)..(5): a, c, t, g, unknown or other
nnnnngagacc

(19) United States
(12) Patent Application Publication
(10) Pub. No.: US 2022/0243212 A1
(43) Pub. Date: Aug. 4, 2022

(54) PRODUCTION OF VECTORS USING PHAGE ORIGIN OF REPLICATION

(71) Applicant: ASKLEPIOS BIOPHARMACEUTICAL, INC., Research Triangle Park, NC (US)

(72) Inventors: Richard Jude SAMULSKI, Research Triangle Park, NC (US); Lester SUAREZ, Research Triangle Park, NC (US)

(73) Assignee: ASKLEPIOS BIOPHARMACEUTICAL, INC., Research Triangle Park, NC (US)

(21) Appl. No.: 17/620,557

(22) PCT Filed: Jun. 19, 2020
(86) PCT No.: PCT/US20/38651

§ 371 Date: Dec. 17, 2021

Related U.S. Application Data
(60) Provisional application No. 62/864,689, filed on Jun. 21, 2019.

(51) Int. Cl. C12N 15/64; C12N 15/86; C12N 9/64

(52) U.S. Cl. CPC C12N 15/64; C12N 15/86; C12N 9/644; C12Y 304/21022; A61K 48/00; C12N 2750/14122; C12N 2750/14151; C12N 2800/10; C12N 2820/60; C12N 2750/14143

(57) ABSTRACT

The present invention provides a method of manufacturing circular nucleic acid vectors containing a transgene comprising: (a) contacting a host system with a template, wherein the template comprises at least one flanking cleavage site(s), and (i) at least one phage origin of replication (ORI); (ii) at least one Terminal Repeat (TR), and; (iii) a promoter sequence operatively linked to a transgene; (b) incubating the host system for a time sufficient for replication to occur resulting in circular nucleic acid production; and (c) recovering the circular nucleic acid production, wherein the circular nucleic acid self-anneals.

### CROSS-REFERENCE TO RELATED APPLICATIONS

This application claims benefit under 35 U.S.C. § 119(e) of the U.S. provisional application No. 62/864,689 filed Jun. 21, 2019, the contents of which are incorporated herein by reference in their entirety.

### FIELD OF THE INVENTION

The present invention relates to methods and cell lines for generating vectors for gene expression.

### BACKGROUND OF THE INVENTION

It is desirable to introduce exogenous DNA to a cell in a manner such that it provides for long-term expression of the protein encoded by the exogenous DNA. Viral based protocols have been developed, in which a viral vector is employed to introduce exogenous DNA into a cell that can subsequently integrate the introduced DNA into the target cell's genome or remain episomally. Viral based vectors finding use include retroviral vectors, e.g., Moloney murine leukemia viral based vectors, adenovirus derived vectors, adeno-associated virus (AAV) derived vectors, HSV derived vectors, sindbis derived vectors, etc. A great deal of interest has focused on the use of AAV vectors. However, methods that are efficient for large scale AAV production remain elusive.

A phagemid makes recombinant displayed protein using a phage-derived origin of replication (ORI). A phage ORI replicates single-stranded circular DNA with very high efficiency. However, phage ORI replication require the additional proteins provided by helper phage to create phage particles that display recombinant protein. Helper phage are essential for phagemid systems as they supply all the other proteins required to make functional phage. Helper phage are normal Ff phages with a number of modifications: they contain an additional origin of replication, they usually carry antibiotic resistance genes and their packaging signal is severely disabled.

When a bacterium is infected with helper phage, the disabled packaging signal does not prevent the production of phage particles. However, when a bacterium is infected with both phagemid and helper phage, the phagemid DNA (containing an optimal packaging signal) is packaged in preference. As a result, phagemid preparations are both phenotypically and genotypically heterogeneous: the display protein may be either wild type (derived from the helper phage) or recombinant (derived from the phagemid), and the packaged genome may be either phage or phagemid. In theory, the disabled packaging signal should significantly reduce the number of helper phage particles in any phagemid preparation. However, the number of helper phage can sometimes equal, or exceed, the number of phagemid particles, which can significantly compromise subsequent selections. Described herein are methods that harness the efficiency of a phagemid for generating a nucleic acid to be utilized in viral production, but do not require helper phage.

### SUMMARY OF INVENTION

One aspect of the invention described herein provides a method of manufacturing circular nucleic acid vectors containing a transgene comprising: (a) contacting a host system with a template, wherein the template comprises at least one flanking cleavage site(s), and within those site(s): (i) at least one phage origin of replication (ORI); (ii) at least one Terminal Repeat (TR), and; (iii) a promoter sequence operatively linked to a transgene; (b) incubating the host system for a time sufficient for replication to occur resulting in circular nucleic acid production; and (c) recovering the circular nucleic acid production, wherein the circular nucleic acid self-anneals.

In one embodiment of any aspect, the template comprises at least two cleavage sites. In one embodiment of any aspect, the template further comprises at least one additional cleavage site immediately downstream of the at least one ORI (see e.g., FIG. 5). In one embodiment of any aspect, the method further comprises the step of cutting at least one cleavage site of the recovered circular nucleic acid (see e.g., FIG. 5).

In one embodiment of any aspect, the method further comprises, following recovery, the step of in vitro replication, e.g., of the circular nucleic acid.

In one embodiment of any aspect, the template further comprises at least one adapter sequence or at least two adapter sequence. In one embodiment of any aspect, the adaptor sequence induces closure of cleaved DNA (see e.g., FIGS. 1-5, 7, and 9). In one embodiment of any aspect, the adaptor sequence further comprises a cleavage site.

In one embodiment of any aspect, the recovered circular nucleic acid is used for delivery of the transgene.

In one embodiment of any aspect, the recovered circular nucleic acid is used for recombinant viral vector production. In one embodiment of any aspect, the viral vector is an adeno associated virus (AAV), a lentivirus (LV), a herpes simplex virus (HSV), an adeno virus (AV), or a pox virus (PV). In one embodiment of any aspect, the vector is a DNA or RNA virus. In one embodiment of any aspect, the virus is an AAV and has a mutant ITR, wherein the mutant ITR is a Double D mutant ITR.

In one embodiment of any aspect, the circular nucleic acid is self-annealed and double-stranded. In one embodiment of any aspect, the vector is single-stranded.

In one embodiment of any aspect, there is a second TR and the promoter sequence operably linked to a transgene is flanked on both sides by a TR.

In one embodiment of any aspect, the ORI is upstream of the left TR. In one embodiment of any aspect, the ORI is flanked by the TRs and upstream of the promoter sequence operably linked to a transgene.

In one embodiment of any aspect, the host system is a bacterial packaging cell. In one embodiment of any aspect, the host system is a cell-free system. In one embodiment of any aspect, the host system is a cell-free system and contains helper phage particles.

In one embodiment of any aspect, the host system is a host cell. Exemplary host cells include a mammalian cell, a bacterial cell, or an insect cell.

In one embodiment of any aspect, the at least one TR is a mutant ITR, a synthetic ITR, a wild-type ITR, or a non-functional ITR.

In one embodiment of any aspect, the vector has flanking DD-ITRs, and in between the flanking is a promoter operatively linked to a sense strand of the transgene, a replication defective ITR, and an anti-sense complement of the transgene.

In one embodiment of any aspect, wherein the ITR is an AAV ITR

In one embodiment of any aspect, the ORI is located upstream of the ITR, and immediately downstream of the upstream ITR.

In one embodiment of any aspect, the at least one phage ORI is an M13 derived ORI, an F1 derived ORI, or an Fd derived ORI.

In one embodiment of any aspect, the template further comprises a second ORI that is a truncated ORI that does not initiate replication. In one embodiment of any aspect, the truncated ORI is ORIΔ29.

In one embodiment of any aspect, the at least two cleavage sites are a restriction site. In one embodiment of any aspect, the at least two restriction sites are identical or different. In one embodiment of any aspect, the restriction site is not found within the transgene sequence.

In one embodiment of any aspect, the cleavage site is cleaved by a nuclease.

In one embodiment of any aspect, the promotor is selected from the group consisting of: a constitutive promoter, a repressible promoter, a ubiquitous promoter, an inducible promoter, a viral promoter, a tissue specific promoter, and a synthetic promoter.

In one embodiment of any aspect, the transgene is a therapeutic gene.

Another aspect of the invention described herein provides a method of manufacturing circular nucleic acid vectors containing a transgene comprising: (a) transforming a host system with a plasmid template, wherein the plasmid template comprises: (i) a phage origin of replication (ORI); (ii) a truncated phage ORI (e.g., ORIΔ29); (iii) at least one Terminal Repeat (TR), and; (iv) a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand; (b) incubating the host system for a time sufficient for replication to occur resulting in circular nucleic acid production; and (c) recovering the circular nucleic acid production, wherein the circular nucleic acid self-anneals.

In one embodiment of any aspect, the plasmid template further comprises a linker and a self-complement linker flanking the ORI.

In one embodiment of any aspect, the transgene contains the sense sequences and the anti-sense complement thereof separated by a linker sequences that will permit the sense and anti-sense strands to bind as a double strand. For example, a linker is a holliday sequence or a replication defective TR.

Another aspect of the invention described herein provides a circular nucleic acid vector manufactured by any of the methods described herein.

Another aspect of the invention described herein provides a circular nucleic acid vector comprising at least one flanking cleavage site(s), and within those site(s): (i) at least one phage origin of replication (ORI); (ii) at least one Terminal Repeat (TR); and (iii) a promoter sequence operatively linked to a transgene.

Yet another aspect of the invention described herein provides a circular nucleic acid vector comprising: (i) a phage origin of replication (ORI); (ii) a truncated phage ORI (e.g., ORIΔ29); (iii) at least one Terminal Repeat (TR), and; (iv) a promoter sequence operatively linked to a transgene, wherein the vector comprises, in the 5′ to 3′ direction, the sense sequence and the anti-sense sequence separated by a hairpin sequence that allows for annealing of the sense and anti-sense strand.

Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “therapeutic gene” refers to a gene or functional fragment thereof encoding a molecule which has a desired therapeutic effect. For example, a gene which either by its absence or mutation causes an increase in pathological cell growth or proliferation of cells. A therapeutic gene as used herein would replace such an absent or mutated gene. Therapeutic genes may give rise to their therapeutic effect either by remaining extrachromosomal such that the gene will be expressed by the cell from the extrachromosomal location or the gene may be incorporated into the genome of the cell such that it recombines with the endogenous gene.

As used herein, “contacting” refers broadly to placing the template or plasmid template into a host system such that it is present in the host system. Less broadly, contacting refers to any appropriate means of placing the template or plasmid template in a host system described herein. Contacting can be by such means that the template is appropriate transported into the interior of the cell such that, e.g., circular nucleic acid is produced by the host cell machinery. Such contacting may involve, for example transformation, transfection, electroporation, or lipofection.

As used herein, the terms “nucleotide sequence”, “nucleic acid sequence”, and “DNA sequence,” are used interchangeably herein and refer to a sequence of a nucleic acid, e.g., a circular nucleic acid that is to be delivered into a target cell. Generally, the nucleic acid comprises an open reading frame that encodes a polypeptide or nontranslated RNA of interest (e.g., for delivery to a cell or subject). The nucleic acid sequence may further comprise regulatory sequences, the combination of which may be referred to as a transgene or expression construct. Preferably the nucleic acid is heterologous, that is not naturally occurring in conjunction with the ITR (e.g. not naturally occurring in a virus from which an ITR is derived). Such a nucleic acid is referred to as heterologous.

As used herein, the term “promoter” refers to a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

As used herein, the term “operably linked” refers to an arrangement of elements wherein the components so described are configured so as to perform their usual function. Thus, a given promoter operably linked to a nucleic acid sequence with a coding sequence is capable of effecting the expression of that sequence when the proper enzymes are present. The promoter need not be contiguous with the sequence, so long as it functions to direct the expression thereof Thus, for example, intervening untranslated yet transcribed sequences can be present between the promoter sequence and the nucleic acid sequence and the promoter sequence can still be considered “operably linked” to a nucleic acid with a coding sequence. Thus, the term “operably linked” is intended to encompass any spacing or orientation of the promoter element and the coding sequence of interest which allows for initiation of transcription of the coding sequence of interest upon recognition of the promoter element by a transcription complex.

As used herein, the term “expression” refers to the cellular processes involved in producing RNA and proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “complement” refers to a DNA sequence having bases that are complementary to that of a given example of DNA, e.g., the template from which its produced from. It is understood that T is complementary to A, and C to G.

As used herein, “self-complementary” refers to a single-strand DNA having a DNA sequence in which the sequence read from the 5′-end and the sequence read from the 3′-end are complementary. Such a sequence can form a double-strand DNA by itself. For example, 5′-GCTTCGATCGAAGC-3′ (SEQ ID NO: 234) is a self-complementary sequence.

As used herein, “plasmid fragment” refers to the double-stranded linear DNA of the plasmid excised via cutting of at least two cleavage sites. For example, the plasmid fragment of the present invention is the single-stranded linear DNA comprising all elements comprised within the at least two cleavage sites, e.., the ORI, the ITRs, and the promoter operatively linked to the transgene. A plasmid fragment is considered a “template” when at least one adaptor is annealed to at least one ends.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

### BRIEF DESCRIPTION OF THE DRAWINGS

FIG. 1 presents a schematic for manufacturing a circular nucleic acid having, in the 5′ to 3′ direction, a BAMHI restriction site, an F1 ORI, an ITR-L, a promotor linked to a transgene (indicated by star), a DD-ITR (mutant), a promotor linked to a transgene (indicated by star), a ITR-R, and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 2 presents a schematic for manufacturing a circular nucleic acid having, in the 5′ to 3′ direction, a BAMHI restriction site, an F1 ORI, an ITR-L, a promotor linked to a transgene (indicated by star), a ITR-R, and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 3 presents a schematic for manufacturing a circular nucleic acid having, in the 5′ to 3′ direction, a BAMHI restriction site, an ITR-L, an F1 ORI, a promotor linked to a transgene (indicated by star), a DD-ITR (mutant), a promotor linked to a transgene (indicated by star), a ITR-R, and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 4 presents a schematic for manufacturing a circular nucleic acid having a, in the 5′ to 3′ direction, BAMHI restriction site, an ITR-L, an F 1 ORI, a promotor linked to a transgene (indicated by star), a ITR-R, and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 5 presents a schematic for manufacturing a circular nucleic acid having, in the 5′ to 3′ direction, a BAMHI restriction site, an F1 ORI, a PVUII restriction site, an ITR-L, a promotor linked to a transgene (indicated by star), a ITR-R, and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells. Following replication, the circular nucleic acid is further cut with a PVUII restriction enzyme, removing an adaptor sequence and the ORI, and resulting in one open end, and one closed end.

FIG. 6 presents a schematic of bioproduction of closed circle linear rAAV genome. The plasmid template is transformed into E. coli cells and undergoes replication. AAV nucleic acid vectors, which are closed circular ssDNA that self-anneals into closed linear DNA, are replicated.

FIG. 7 presents a schematic of manufacturing a vector having, in the 5′ to 3′ direction, a Sfi1 or PvuII restriction site, an F1 ORI, an ITR-L, a promotor linked to a transgene (indicated by star), a DD-ITR (mutant), a promotor linked to a transgene (indicated by star), a ITR-R, and a second Sfi1 or PvuII restriction site, with adaptor sequences ligated to the end via the restriction sites. Prior to ligation of the adaptor sequences, the vector is excised via cutting with a Sfi1 or PvuII restriction enzyme. This vector can be replicated in vitro, e.g., in bacterial packaging cells.

FIG. 8 presents a schematic of manufacturing a self-complementary, single stranded DNA vector having in the 5′ to 3′ direction, an F1 ORI, an ITR-L, a promotor linked to a transgene (indicated by star), a DD-ITR (mutant), a promotor linked to a transgene (indicated by star), a ITR-R, a hairpin sequence, a complementary ITR-R, a complementary promotor linked to a transgene (indicated by star), a complementary DD-ITR (mutant), a complementary promotor linked to a transgene (indicated by star), a complementary ITR-L, and a ORIΔ29. This method uses a bacterial packaging cell and a helper phage. Asterisk indicates a complementary sequence, e.g., a complementary TR or transgene sequence.

FIG. 9 presents a schematic of generating a single stranded vector. (1) Shows a vector having flanking PvuII restriction site, an F1 ORI (e.g., M13), ITRs, including at least one double stranded ITR with adaptor sequences ligated to the end via the restriction sites. The plasmid is cut with a PvuII restriction enzyme and adaptor sequences are annealed, circularizing the DNA. (2) Shows that intermediate dimers from viral genome replication in a host cell of template with an M13 ORI can also be isolated by Hirt extraction, used as a template for more replication, and used for rAAV viral production or in vivo delivery of transgene. A Double D ITR (DD-TR) is the preferred substrate. (3) Indicates downstream in vivo applications can be performed.

FIG. 10 presents a schematic for manufacturing a circular nucleic acid having, in the 5′ to 3′ direction, a BAMHI restriction site, an F1 ORI, an DD-ITR (mutant), a promotor linked to a transgene (indicated by star), a DD-ITR (mutant), a promotor linked to a transgene (indicated by star), a DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

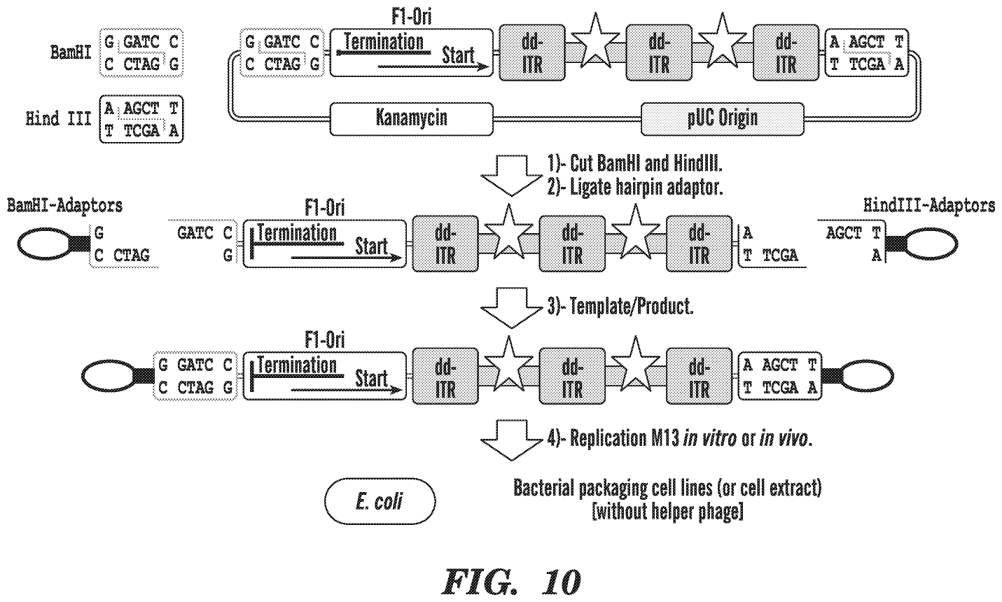

FIG. 11 presents a schematic for manufacturing a circular nucleic acid having, in the 5′ to 3′ direction, a BAMHI restriction site, an F1 ORI, an DD-ITR (mutant), a promotor linked to a transgene (indicated by star), a DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 12 presents a schematic for manufacturing a circular nucleic acid having, in the 5′ to 3′ direction, a BAMHI restriction site, an DD-ITR (mutant), an F1 ORI, a promotor linked to a transgene (indicated by star), a DD-ITR (mutant), a promotor linked to a transgene (indicated by star), a DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 13 presents a schematic for manufacturing a circular nucleic acid having a, in the 5′ to 3′ direction, BAMHI restriction site, an DD-ITR (mutant), an F1 ORI, a promotor linked to a transgene (indicated by star), a DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells.

FIG. 14 presents a schematic for manufacturing a circular nucleic acid having, in the 5′ to 3′ direction, a BAMHI restriction site, an F1 ORI, a PVUII restriction site, an DD-ITR (mutant), a promotor linked to a transgene (indicated by star), DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells. Following replication, the circular nucleic acid is further cut with a PVUII restriction enzyme, removing an adaptor sequence and the ORI, and resulting in one open end, and one closed end.

FIG. 15 presents a schematic of manufacturing a vector having, in the 5′ to 3′ direction, a Sfi1 or PvuII restriction site, an F1 ORI, an DD-ITR (mutant), a promotor linked to a transgene (indicated by star), a DD-ITR (mutant), a promotor linked to a transgene (indicated by star), a DD-ITR (mutant), and a second Sfi1 or PvuII restriction site, with adaptor sequences ligated to the end via the restriction sites. Prior to ligation of the adaptor sequences, the vector is excised via cutting with a Sfi1 or PvuII restriction enzyme. This vector can be replicated in vitro, e.g., in bacterial packaging cells.

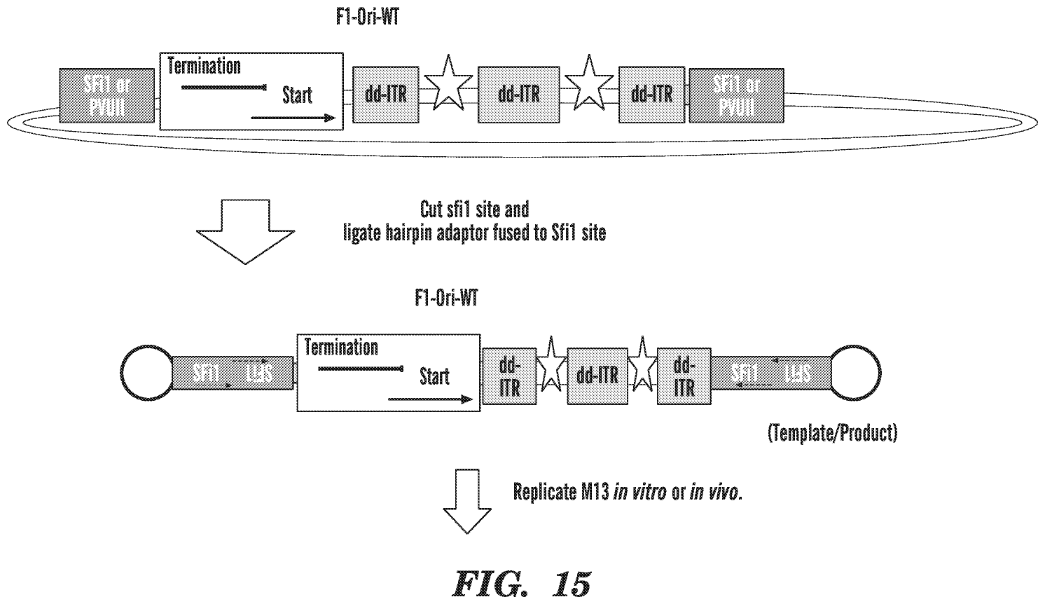

FIG. 16 presents a schematic of manufacturing a self-complementary, single stranded DNA vector having in the 5′ to 3′ direction, an F1 ORI, an DD-ITR (mutant), a promotor linked to a transgene (indicated by star), a DD-ITR (mutant), a promotor linked to a transgene (indicated by star), a DD-ITR (mutant), a hairpin sequence, a complementary DD-ITR (mutant), a complementary promotor linked to a transgene (indicated by star), a complementary DD-ITR (mutant), a complementary promotor linked to a transgene (indicated by star), a complementary DD-ITR (mutant), and a ORIΔ29. This method uses a bacterial packaging cell and a helper phage. Asterisk indicates a complementary sequence, e.g., a complementary TR or transgene sequence.

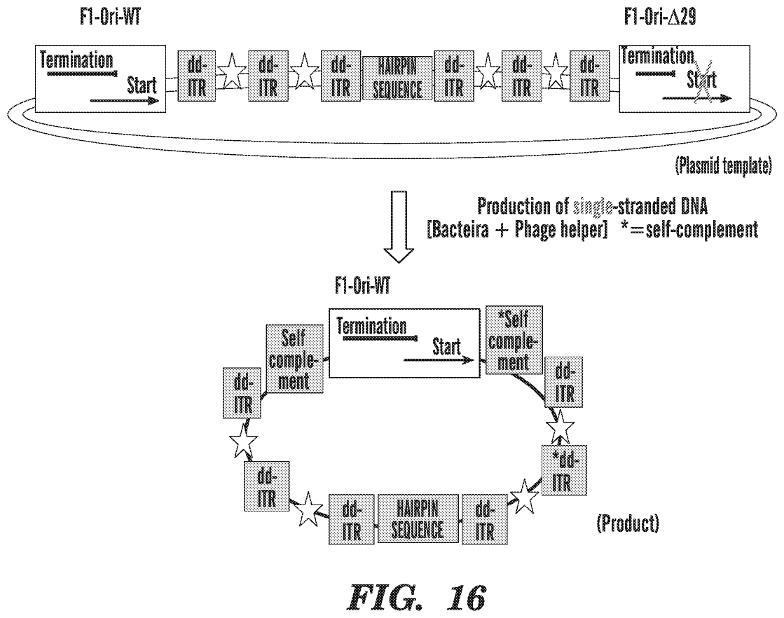

### DETAILED DESCRIPTION OF THE INVENTION

One aspect of the invention described herein provides a method of manufacturing circular nucleic acid vectors containing a transgene comprising: (a) contacting a host system with a template, wherein the template comprises at least one flanking cleavage site(s), and (i) at least one phage origin of replication (ORI); (ii) at least one Terminal Repeat (TR), and; (iii) a promoter sequence operatively linked to a transgene; (b) incubating the host system for a time sufficient for replication to occur resulting in circular nucleic acid production; and (c) recovering the circular nucleic acid production, wherein the circular nucleic acid self-anneals.

Another aspect of the invention described herein provides a method of manufacturing circular nucleic acid vectors containing a transgene comprising: (a) transforming a host system with a plasmid template, wherein the plasmid template comprises: (i) a phage origin of replication (ORI); (ii) a truncated phage ORI (e.g., ORIΔ29); (iii) at least one Terminal Repeat (TR), and; (iv) a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand; (b) incubating the host system for a time sufficient for replication to occur resulting in circular nucleic acid production; and (c) recovering the circular nucleic acid production, wherein the circular nucleic acid self-anneals.

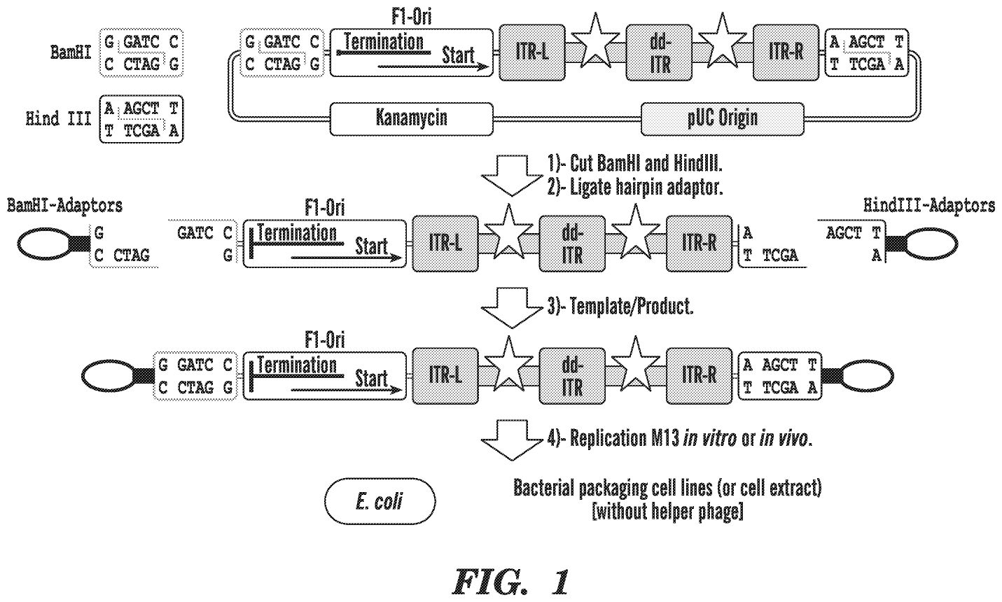

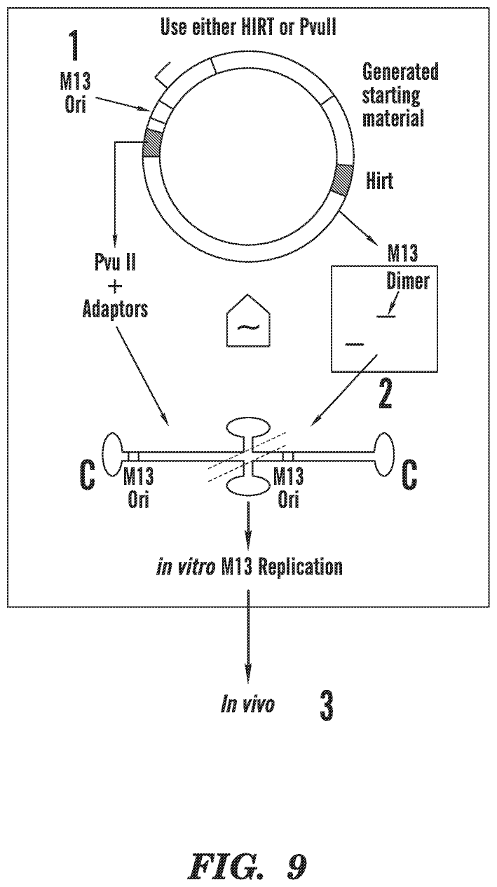

In one embodiment, a template used to produce circular nucleic acids is generated by cutting double-stranded plasmid DNA which comprises the components of the template (for example, see FIGS. 1-5, 7, and 9) with a nuclease that specifically targets the cleavage site present on the plasmid, e.g., a restriction enzyme. In an alternative embodiment, double-stranded plasmid template can be used to produce circular nucleic acids. A plasmid comprising components of a template, or a plasmid template described herein can be generated using standard cloning techniques known in the art. Cutting of the cleavage sites excises the plasmid fragment, i.e., a single-stranded linear DNA found between the two cleavage sites. In one embodiment, the plasmid fragment is than annealed to adaptor proteins at the cut ends. For example, if a cleavage site is cut with a restriction enzyme, “sticky” ends (i.e., an end of a DNA double helix at which a few unpaired nucleotides of one strand extend beyond the other) are produced. An adaptor sequence having a complementary sticky end would be capable of annealing to the sticky end using standard techniques known in the art, for example, a ligation reaction using a T4 ligase and ATP. Annealing the adaptor sequences to the ends of the plasmid fragment circularizes the DNA, creating a closed-end DNA structure, referred to herein as a template.

A template can be replicated in vitro or in vivo in a host system. For example, in E. coli cells using standard methods, e.g., as described in Shepherd, et al. Scientific Reports 9, Article number: 6121 (2019); cell extracts, e.g., E. coli cell extracts, as described in Wang, G., et al. Cell Research 7, 1-12(1997); or in a bacterial packaging cell line known in the art (the contents of these citations are incorporated herein by reference in their entireties). A bacterial packaging cell line can express M13-based helper plasmids, e.g., as described in Chastenn, L., et al. Nucleic Acids Res. 2006 December; 34(21): e145, the contents of which are incorporated herein by reference in its entirety.

Alternatively, a template described herein need not undergo replication, and can be used to directly contact a host system, for example, an in vitro cell line.

The phage ORI located on the template initiates the replication of a single-stranded, complementary circle DNA, referred to herein as circular nucleic acid. In one embodiment, the template is incubated in the host system for a time sufficient to replicate circular nucleic acid. In one embodiment, the phage ORI initiates replication without requiring any additional components, e.g., helper phage. In an alternative embodiment, phage ORI-initiated replication occurs in the presence of additional components, e.g., helper phage. A host system used for replication of the circular nucleic acid can be, e.g., an in vitro or in vivo host system.

In one embodiment, the template is single-stranded and in vitro or in vivo replication of the template generates single-stranded circular nucleic acids. The single-stranded circular DNA can self-anneal, for example, at the transgene sequence, and become double stranded.

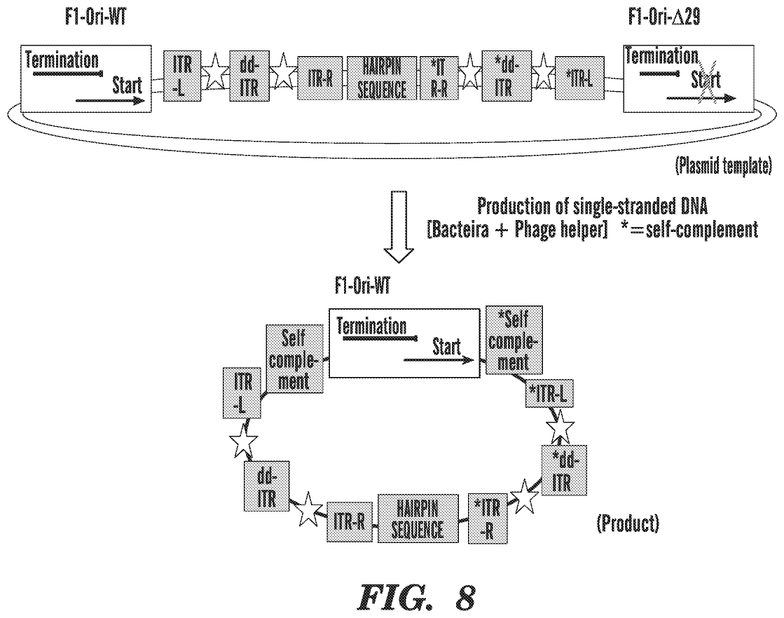

When an ORI is present on both sides of the plasmid template, for example, a plasmid template having an F1 ORI and a ORIΔ29 flanking the other elements of the template (see, e.g., FIG. 8), the single-stranded circular nucleic acid contains a self-complementary transgene, e.g., a therapeutic transgene. In one embodiment, the single-stranded circular nucleic acid contains the sense sequence of the transgene and the anti-sense sequence of the transgene on one strand. In one embodiment, the sense and the anti-sense sequences are separated by a linker, e.g., a Holliday linker or a defective ITR, that allows the strand to bend and binding of the sense and antisense sequences to occur. It is specifically contemplated herein that the linker can be any sequence that allows for the bending of the strand that facilitates the binding of the sense and anti-sense sequences of the transgene. In one embodiment, the single-stranded circular nucleic acids further comprise a complement region and self-complement region flanking the ORI. See, e.g., FIG. 8.

Circular nucleic acids are released (i.e., set free) from the host system using standard techniques known for a specific host system, such as mechanical-mediated release (sonication) or chemical-mediated release (detergents). Following release, circular nucleic acids are recovered using standard methods, for example, via purification using column chromatography.

A circular nucleic acid generated herein can be closed-ended, open-ended, or both open-ended and closed ended. In one embodiment, the circular nucleic acid is closed-ended. A closed-ended DNA vector can have any configuration, for example, doggie bone, dumbbell, circular, closed-ended linear duplex, etc.

In one embodiment, the circular nucleic acid contains at least a third, unique cleavage site downstream and adjacent to the ORI. Following replication of the circular nucleic acid, this unique cleavage site can be cut, removing the ORI from the circular nucleic acid and resulting in an open end. This nucleic acid is both open-ended and close-ended. An open- and closed-ended nucleic can be administered to a subject for, e.g., gene delivery via transgene expression.

A circular nucleic acid replicate generated using methods described herein can be used for delivery of the transgene it expresses, or to generate more circular nucleic acids, e.g., via additional in vitro or in vivo replication. A circular nucleic acid replicate can additionally be used in recombinant viral vector production, e.g., for the production of an adeno-associated viral vector in an HEK293 cell.

Further, a circular nucleic acid can be packaged, e.g., into a capsid or liposome, for use in downstream applications.

In one embodiment, circular nucleic acids manufactured using methods described herein can be used in the production of recombinant vectors, e.g., a recombinant viral vector. By way of example, the circular nucleic acid having at least one ITR can be used in place of a plasmid expressing the at least one ITR in the production of an AAV vector. Replication of AAV genome using a template recombinant plasmid is further discussed in, for example, Samulski, R J, et al. Journal of Viol. October 1987, the contents of which are incorporated herein by reference in its entirety. Protocols for producing recombinant vectors and for using vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Further, production of AAV vectors is further described, e.g., in U.S. Pat. No. 9,441,206, the contents of which is incorporated herein by reference in its entirety.

Non-limiting examples of vectors employed in the methods of this invention include any nucleotide construct used to deliver nucleic acid into cells, e.g., a plasmid, a template, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus vector can then be used to infect and thereby deliver a therapeutic transgene of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). Incubation conditions (e.g., timing, climate, medium, etc.) for a given condition are known in the art and can be readily identified by a skilled practitioner.

Viral vectors produced in a cell can be released (i.e. set free from the cell that produced the vector) using any standard technique. For example, viral vectors can be released via mechanical methods, for example microfluidization, centrifugation, or sonication, or chemical methods, for example lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

In one embodiment, the vector can be, but is not limited to a nonviral vector or a viral vector. In one embodiment of any aspect, the vector is a DNA or RNA virus. Non-limiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors produced by the method of the invention may comprise the genome, in part or entirety, of any naturally occurring and/or recombinant viral vector nucleotide sequence (e.g., AAV, AV, LV, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

Variant viral vector sequences can be used to produce viral vectors in the host system described herein. For example, or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, AV, LV, etc.).

It is to be understood that a viral expression system will further be modified to include any necessary elements required to complement a given viral vector during its production using methods described herein. For example, in certain embodiment, the nucleic acid cassette is flanked by terminal repeat sequences. In one embodiment, for the production of rAAV vectors, the AAV expression system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

A viral expression system for manufacturing an AAV vector (e.g., an AAV expression system) could further comprise Replication (Rep) genes and/or Capsid (Cap) genes in trans, for example, under the control of an inducible promoter. Expression of Rep and Cap can be under the control of one inducible promoter, such that expression of these genes are turned “on” together, or under control of two separate inducible promoters that are turned “on” by distinct inducers. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties.

In one embodiment, the cells for producing an AAV vector are cultured in suspension. In another embodiment, the cells are cultured in animal component-free conditions. The animal component-free medium can be any animal component-free medium (e.g., serum-free medium) compatible with a given cell line, for example, HEK293 cells. Any cell line known in the art to be capable of propagating an AAV vector can be used for AAV production using methods described herein. Exemplary cell lines that can be used to generate an AAV vector include, without limitation, HEK293, CHO, Cos-7, and NSO.

In one embodiment, a cell line for producing an AAV vector stably expresses any of the components required for AAV vector production, e.g., Rep, Cap, VP1, etc. In one embodiment, a cell line for producing an AAV vector transiently expresses any of the components required for AAV vector production, e.g., Rep, Cap, VP1, etc.

In the event that a cell line for producing an AAV vectors does not stably or transiently express rep or cap, these sequences are to be provided to the AAV expression system, e.g., via a circular nucleic acid produced using methods described herein. AAV rep and cap sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV rep/ cap sequences will not be flanked by the TRs, to prevent rescue and/or packaging maintain of these sequences.

A viral expression system for manufacturing a lentivirus using methods described herein would further comprise long terminal repeats (LTRs) flanking the nucleic acid cassette. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase the host chromosome. LTRs and methods for manufacturing lentiviral vectors are further described, e.g., in U.S. Pat. Nos. 7,083,981B2; 6,207,455B1; 6,555,107B2; 8,349,606B2; 7,262,049B2; and U.S. Patent Application Numbers US20070025970A1; US20170067079A1; US20110028694A1; the contents of each are incorporated herein by reference in their entireties.

A viral expression system for manufacturing an adenovirus using methods described herein would further comprise identical Inverted Terminal Repeats (ITR) of approximately 90-140 base pairs (exact length depending on the serotype) flanking the nucleic acid cassette. The viral origins of replication are within the ITRs exactly at the genome ends. The adenovirus genome is a linear double-stranded DNA molecule of approximately 36000 base pairs. Often, adenoviral vectors used in gene therapy have a deletion in the E1 region, where novel genetic information can be introduced; the E1 deletion renders the recombinant virus replication defective. ITRs and methods for manufacturing adenovirus vectors are further described, e.g., in U.S. Pat. Nos. 7,510,875B2; 7,820,440B2; 7,749,493B2; 7,820,440B2; 10,041,049B2; International Patent Application Numbers WO2000070071A1; and U.S. Patent Application Numbers WO2000070071A1; US20030022356A1; US20080050770A1 the contents of each are incorporated herein by reference in their entireties.

In one embodiment, the viral expression system can be a host cell, such as a virus, a mammalian cell or an insect cell. Exemplary insect cells include but are not limited to Sf9, Sf21, Hi-5, and S2 insect cell lines. For example, a viral expression system for manufacturing an AAV vector could further comprise a baculovirus expression system, for example, if the viral expression system is an insect cell. The baculovirus expression system is designed for efficient large-scale viral production and expression of recombinant proteins from baculovirus-infected insect cells. Baculovirus expression systems are further described in, e.g., U.S. Pat. Nos. 6,919,085B2; 6,225,060B1; 5,194,376A; the contents of each are incorporated herein by reference in their entireties.

In another embodiment, the viral expression system is a cell-free system. Cell-free systems for viral vector production are further described in, for example, Cerqueira A., et al. Journal of Virology, 2016; Sheng J., et al. The Royal Society of Chemistry, 2017; and Svitkin Y. V., and Sonenberg N. Journal of Virology, 2003; the contents of which are incorporated herein by reference in their entireties.

One aspect provided herein is a vector manufactured using any of the methods described herein.

Another aspect provided herein is a circular nucleic acid vector comprising: at least one flanking cleavage sites, and (i) at least one phage origin of replication (ORI); (ii) at least one Terminal Repeat (TR), and; (iii) a promoter sequence operatively linked to a transgene.

Yet another aspect provided herein is a circular nucleic acid vector comprising: (i) a phage origin of replication (ORI); (ii) a truncated phage ORI (e.g., ORIΔ29); (iii) at least one Terminal Repeat (TR), and; (iv) a promoter sequence operatively linked to a transgene, wherein the vector comprises, in the 5′ to 3′ direction, the sense sequence and the anti-sense sequence separated by a hairpin sequence that allows for annealing of the sense and anti-sense strand.

It is understood that a host system would further comprise components necessary for a given vector. For example, production of an AAV requires the presence of at least one Replication (Rep) genes and/or at least Capsid (Cap) genes. In one embodiment, the vector is an AAV and the host system constitutively expresses at least one Replication (Rep) genes and/or at least Capsid (Cap) genes. In another embodiment, the vector is an AAV and a nucleic acid expressing at least on Rep gene and a nucleic acid expressing at least one Cap gene are transformed in the host system prior to step (a) of the method described herein, or co-transformed with step (a) of the method described herein. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties. In various embodiments, nucleic acids expressing Rep and/or Cap genes are transformed using standard methods, for example, by a plasmid, a virus, a liposome, a microcapsule, a non-viral vector, or as naked DNA.

In one embodiment, the host system can be a host cell, such as an insect cell, a mammalian cell, a virus, or a bacterial packaging cell. For example, a host system for manufacturing an AAV vector could further comprise a baculovirus expression system, for example, if the host system is an insect cell. The baculovirus expression system is designed for efficient large-scale viral production and expression of recombinant proteins from baculovirus-infected insect cells. Baculovirus expression systems are further described in, e.g., U.S. Pat. Nos. 6,919,085B2; 6,225,060B1; 5,194,376A; the contents of each are incorporated herein by reference in their entireties. Exemplary insect cells include but are not limited to Sf9, Sf21, Hi-5, and S2 insect cell lines.

In another embodiment, the host system is a cell-free system. For example, the vectors can be synthesized and assembled in an in vitro system. One can prepare cassettes that will express the necessary enzymatic protein, e.g., for lentivirus, pol; for AAV, Rep. In one embodiment, the cell-free system comprises helper phage particles. Helper phage particles, for example, M13K07, provide the necessary gene products for particle formation when using phage vectors. Helper phage particles are further reviewed in, for example, in (2005) Helper Phage. In: Encyclopedic Reference of Genomics and Proteomics in Molecular Medicine. Springer, Berlin, Heidelberg; the contents of which are incorporated herein by reference in its entirety.

Other cassettes can be assembled that will express the necessary structural proteins, e.g., for lentivirus, gag and env; for AAV, the cap gene that expresses VP1, VP2, and VP3. Another vector will be synthesized having a gene operably linked to the desired transgene, which is ultimately flanked between packaging sequences, such as a LTR or an ITR. Various methods to accomplish this are known in the art. Cell-free systems for vector production are further described in, for example, Cerqueira A., et al. Journal of Virology, 2016; Sheng J., et al. The Royal Society of Chemistry, 2017; and Svitkin Y.V., and Sonenberg N. Journal of Virology, 2003; the contents of which are incorporated herein by reference in their entireties.

Origin of Replication

Templates described herein comprise at least one origin of replication (ORI), i.e., the site in which replication is initiated, derived from filamentous phage (Ff phage). A filamentous phage ORI is a region of the phage genome, as is well known, that defines sites for initiation of replication, termination of replication and packaging of the replicative form produced by replication. A plasmid having an ORI derived only from phage, i.e., does not comprise an ORI derived from an organism other than phage, is known as a phagemid. Replication of a phagemid via a filamentous ORI is further reviewed in, e.g., Specthrie, L, et al. Journal of Mol Biol. V. 228(3), 1992; and Nafisi, P M, et al. Synthetic Biol. 2018, the contents of each of which are incorporated herein by reference in their entireties. Suitable filamentous phage ORI for use in the present invention is a M13, f1 or fd phage origin of replication.

Use of a phage ORI described herein is advantageous as it does not necessarily require the presence of a helper phage to initiate replication, eliminating the likelihood of helper phage contamination in the replicate. Phage ORIs described herein independently initiate replication of single-stranded circle, i.e., circular nucleic acids.

The ORI of the present invention is not limited with respect to its location on the template. An ORI can be located upstream or downstream of the at least one ITR or the at least one cleavage site. In one embodiment, the ORI is upstream of the left TR. In one embodiment, the ORI is flanked by the TRs and upstream of the promoter sequence operable linked to a transgene.

In one embodiment, the template contains an F1 ORI. F1 is a phage-derived ORI that allows for the replication and packaging of ssDNA into phage particles. In one embodiment, the ORI derived from F1 has the nucleotide sequence of SEQ ID NO: 235.

(SEQ ID NO: 235)
ACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCT
CGACCCCAAAAAACTTGATTAGGGTGATGGTTCACGTAGTGGGCCATCG
CCCTGATAGACGGTTTTTCGCCCTTTGACGTTGGAGTCCACGTTCTTTA
ATAGTGGACTCTTGTTCCAAACTGGAACAACACTCAACCCTATCTCGGT
CTATTCTTTTGATTTATAAGGGATTTTGCCGATTTCGGCCTATTGGTTA
AAAAATGAGCTGATTTAACAAAAATTTAACGCGAATTA

In another embodiment, the ORI is derived from M13. The M13 ORI facilitates M13 helper-dependent replication of the template. In one embodiment, the ORI derived from M13 has the nucleotide sequence of SEQ ID NO: 236.

(SEQ ID NO: 236)
ACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCT
CGACCCCAAAAAACTTGATTTGGGTGATGGTTCACGTAGTGGGCCATCG
CCCTGATAGACGGTTTTTCGCCCTTTGACGTTGGAGTCCACGTTCTTTA
ATAGTGGACTCTTGTTCCAAACTGGAACAACACTCAACCCTATCTCGGG
CTATTCTITTGATTTATAAGGGATTTTGCCGATTTCGG

In one embodiment, the at least one ORI includes a second ORI that is mutated as compared to a wild-type ORI. A mutated ORI can comprise single nucleotide mutations, e.g., nucleotide deletion, insertion, or substitutions) or can be truncated to lack at least a portion (e.g., at least five nucleotides) of the wild-type ORI sequence. A mutant ORI can be a non-functional ORI. For example, a non-functional ORI would have reduced or a complete loss of the function of a wild-type ORI, e.g., initiate replication.

In one embodiment, the mutant ORI is a mutant F1 ORI, F1-ORIΔ29. Mutant ORIΔ29 is a truncated F1 ORI that lacks the capacity to initiate replication. ORIΔ29 is further reviewed in, e.g., Specthrie, L, et al. Journal of Mol Biol. V. 228(3), 1992. In one embodiment, ORIΔ29 has the nucleotide sequence of SEQ ID NO: 237.

(SEQ ID NO: 237)
ACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCT
CGACCCCAAAAAACTTGATTAGGGTGATGGTTCACGTAGTGGGCCATCG
CCCTGATAGACGGTTTTTCGCCCTTTGACGTTGGAGTCCACGTTCTTTA
ATAGTGGACTCTTGTTCCAAACTGGTTTAACACTC

In one embodiment, the mutant ORI is a mutant M13 ORI, M13-ORIΔ29. Mutant ORIΔ29 is a truncated M13 ORI that lacks the capacity to initiate replication. In one embodiment, ORIΔ29 has the nucleotide sequence of SEQ ID NO: 238.

(SEQ ID NO: 238)
ACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCT
CGACCCCAAAAAACTTGATTTGGGTGATGGTTCACGTAGTGGGCCATCG
CCCTGATAGACGGTTTTTCGCCCTTTGACGTTGGAGTCCACGTTCTTTA
ATAGTGGACTCTTGTTCCAAACTGGTTTAACACTC

Circular nucleic acids described herein do not comprise other types or species of ORI, for example, the vector does not comprise bacterial or mammalian ORI.

In certain instances, the ORI is cut out of the template following replication. In one embodiment, the ORI is flanked by at least two cleavage sites, i.e., a cleavage site is located just upstream of the ORI, and a second cleavage site is located just downstream of the ORI. A template having this configuration is cut following replication to remove the ORI from the template. It is specifically contemplated herein that a template for use in transgene delivery to a subject would not comprise a phage ORI.

Certain ORIs require that additional cellular components be present in order to initiate replication. For example, the M13 ORI requires a M13-derived helper phage. In one embodiment, the phage derived ORI requires the presence of a helper phage, for example during in vitro replication of a single-stranded template. In one embodiment, a host system transiently expresses a helper phage. For example, a helper phage can be expressed in a host system prior to, following, or at substantially the same time as the template expression. In an alternative embodiment, the host system constitutively expresses the helper phage. One skilled in the art will be able to assess whether additional components, e.g., helper genes, are required for initiation of replication at a particular ORI.

Terminal Repeats

The template described herein comprises at least one terminal repeat (TR), e.g., an inverted terminal repeat (ITR). For example, the template can comprise at least 1, at least 2, at least 3, at least 4, at least 5, or more TR. In one embodiment, the there is a second TR and the promoter sequence operably linked to a transgene is flanked on both sides by a TR.

In various embodiments, the TR is an ITR. An ITR includes any viral terminal repeat or synthetic sequence that forms a hairpin structure and functions as an inverted terminal repeat (i.e., mediates the desired functions such as replication, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, e.g., as described in U.S. Pat. No. 9,169,494, the contents of which are incorporated by reference in their entirety. Typically, the ITR is 145 nucleotides. The terminal 125 nucleotides form a palindromic double stranded T-shaped hairpin structure. In the structure the A-A′ palindrome forms the stem, and the two smaller palindromes B-B′ and C-C′ form the cross-arms of the T. The other 20 nucleotides in the D sequence remain single-stranded. In the context of an AAV genome, there would be two ITR's, one at each end of the genome.

An AAV ITR may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered. An AAV ITR need not have the native terminal repeat sequence (e.g., a native AAV ITR sequence may be altered by insertion, deletion, truncation and/or missense mutations), as long as the terminal repeat mediates the desired functions, e.g., replication, or, integration.

In one embodiment, the ITR is a wild-type ITR. In another embodiment, the ITR is a mutant ITR. A mutant ITR can be a non-functional ITR. For example, a non-functional ITR would have reduced or a complete loss of the function of a wild-type ITR, e.g., mediates replication, integration and/or provirus rescue.

In one embodiment, the mutant ITR is a DD mutant ITR (DD-ITR). A DD-ITR has the same sequence the ITR from which it is derived, but includes a second D sequence adjacent the A sequence, so there are D and D′. The D and D′ can anneal (e.g., as described in U.S. Pat. No. 5,478,745, the contents of which are incorporated herein by reference). Each D is typically about 20 nt in length, but can be as small as 5 nucleotides. Shorter D regions preserve the A-D junction (e.g., are generated by deletions at the 3′ end that preserve the A-D junction). Preferably the D region retains the nicking site and/or the A-D junction. The DD-ITR is typically about 165 nucleotides. The DD-ITR has the ability to provide information in cis for replication of the DNA construct. Thus, a DD-ITR has an inverted palindromic sequence with flanking D and D′ elements, e.g. a (+) strand 5′to 3′ sequence of 5′-DABB′CC′A′D′-3′ and a (−) strand complimentary to the (+) strand that has a 5′ to 3′ sequence of 5′ -DACC′BB′A′D′-3′ that can form a Holiday structure, e.g. as illustrated in FIG. 1. In certain embodiments, the DD-ITR may have deletions in its components (e.g. A-C), while still retaining the D and D′ element. In certain embodiments, the ITR comprises deletions while still retaining the ability to form a Holliday structure and retaining two copies of the D element (D and D′). The DD-ITR can be generated from a native AAV ITR or from a synthetic ITR. In certain embodiments, the deletion is in the B region element. In certain embodiments, the deletion is in the C region element. In certain embodiments, a deletion within both the B and C element of the ITR. In one embodiment, the entire B and/or C element is deleted, and e.g. replaced with a single hairpin element. In one embodiment, the template comprises at least two DD-ITRs.

A synthetic ITR refers to a non-naturally occurring ITR that differs in nucleotide sequence from wild-type ITRs, e.g., the AAV serotype 2 ITR (ITR2) sequence due to one or more deletions, additions, substitutions, or any combination thereof The difference between the synthetic and wild-type ITR (e.g., ITR2) sequences may be as little as a single nucleotide change, e.g., a change in 1, 2, 3, 4, 5, 6, 7, 8, 9, 10, 15, 20, 25, 30, 35, 40, 45, 50, 60, 60, 70, 80, 90, or 100 or more nucleotides or any range therein. In some embodiments, the difference between, the synthetic and wild-type ITR (e.g., ITR2) sequences may be no more than about 100, 90, 80, 70, 60, 50, 45, 40, 35, 30, 25, 20, 15, 10, 9, 8, 7, 6, 5, 4, 3, 2, or 1 nucleotide or any range therein.

Additional TRs can be used in the current invention, for example a long terminal repeat (LTR).

In one embodiment, the ITRs present on the template can be used, for example, for the production of an AAV vector. Methods for producing AAV vectors are described herein above.

Cleavage Site

A template described herein comprises at least one cleavage sites, which flank other elements of the template. A cleavage site is a nucleotide sequence in which the phosphodiester backbone is selectively broken. For example, a nucleotide sequence recognized by a nuclease is a cleavage site because the enzyme will cut the phosphodiester backbone at selective sites within the sequence. Such cleavage sites may be single or double-stranded, depending on the endonuclease. Also included are chemical cleavage sites such as pyrimidine and purine cleavage reactions performed in Maxam and Gilbert sequencing, or cleavage through chemical methods such as oxidation as described in U.S. Pat. No. 4,795,700, which is incorporated herein by reference

In one embodiment, the template further comprises at least a second cleavage, and within the sites are the additional elements contained on the template, e.g., at least on ORI, at least one TR, and promotor operatively linked to a therapeutic transgene, such that the at least two cleavage sites flank these elements. In one embodiment, a third cleavage site is located immediately downstream of the ORI.

In one embodiment, the cleavage site is cut by a nuclease. As used herein, the term “nuclease” refers to molecules which possesses activity for DNA cleavage. Particular examples of nuclease agents include zinc finger proteins, meganucleases, TAL domains, TALENs, yeast assembly, recombinases, leucine zippers, CRISPR/Cas, endonucleases, and other nucleases known to those in the art. Nucleases can be selected or designed for specificity in cleaving at a given target site, e.g., a cleavage site. For example, nucleases can be selected for cleavage at a target site that create overlapping ends between the cleaved polynucleotide and a different polynucleotide. As used herein, the term “recognition site for a nuclease” refers to a DNA sequence at which a nick or double-strand break is induced by a nuclease.

In one embodiment, the nuclease is protelomerase and the cleavage site is a protelomerase target sequence, e.g., the TelN recognition site. A protelomerase target sequence is any DNA sequence whose presence in a DNA template allows for its conversion into a closed linear DNA by the enzymatic activity of protelomerase. In other words, the protelomerase target sequence is required for the cleavage and religation of double stranded DNA by protelomerase to form covalently closed linear DNA.

Typically, a protelomerase target sequence comprises any perfect palindromic sequence i.e., any double-stranded DNA sequence having two-fold rotational symmetry, also described herein as a perfect inverted repeat. As shown in U.S. Pat. No. 9,109,250, the contents of which are incorporated by reference in their entirety, the protelomerase target sequences from various mesophilic bacteriophages, and a bacterial plasmid all share the common feature of comprising a perfect inverted repeat. The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g., E. coli N15, the central perfect inverted palindrome is flanked by inverted repeat sequences, i.e., forming part of a larger imperfect inverted palindrome.

In one embodiment, the protelomerase has a sequence of SEQ ID NO: 239.

SEQ ID NO: 239 is a nucleotide sequence of a protelomerase.

(SEQ ID NO: 239)
TATCAGCACACAATTGCCCATTATACGCGCGTATAATGGACTATTGTGT
GCTGATA

In one embodiment, the nuclease is a restriction endonuclease and the cleavage site is a recognition site for the endonuclease (i.e., a restriction site). Restriction endonucleases are hydrolytic enzymes capable of catalyzing site-specific cleavage of DNA molecules. The locus of restriction endonuclease action is determined by the existence of a specific nucleotide sequence. Such a sequence is termed the recognition site for the restriction endonuclease. Restriction endonucleases from a variety of sources have been isolated and characterized in terms of the nucleotide sequence of their recognition sites (i.e., restriction site). Some restriction endonucleases hydrolyze the phosphodiester bonds on both strands at the same point, producing blunt ends. Others catalyze hydrolysis of bonds separated by a few nucleotides from each other, producing free single-stranded regions at each end of the cleaved molecule. Such single-stranded ends are self-complementary, hence cohesive, and may be used to rejoin the hydrolyzed DNA. Since any DNA susceptible of cleavage by such an enzyme must contain the same recognition site, the same cohesive ends will be produced, so that it is possible to join heterologous sequences of DNA which have been treated with a restriction endonuclease to other sequences similarly treated. See Roberts, R. J., Crit. Rev. Biochem. 4, 123 (1976). Restriction sites are relatively rare, however the general utility of restriction endonucleases has been greatly amplified by the chemical synthesis of double stranded oligonucleotides bearing the restriction site sequence. Therefore, virtually any segment of DNA can be coupled to any other segment simply by attaching the appropriate restriction oligonucleotide to the ends of the molecule, and subjecting the product to the hydrolytic action of the appropriate restriction endonuclease, thereby producing the requisite cohesive ends. See Heyneker, H. L., et al., Nature 263, 748 (1976) and Scheller, R. H., et al., Science 196, 177 (1977). An important feature of the distribution of restriction endonuclease recognition sites is that they are randomly distributed with respect to reading frame. Consequently, cleavage by restriction endonuclease may occur between adjacent codons or it may occur within a codon.

Restriction sites can be classified by the number of bases in its recognition site, e.g., usually between 4 and 8 bases. The number of bases in the sequence will determine how frequent the site will appear by chance in any given genome, e.g., a 4-base pair sequence would theoretically occur once every 4⁴ or 256 bp, 6 bases, 4⁶ or 4,096 bp, and 8 bases would be 4⁸ or 65,536bp. Restriction sites are often palindromic, meaning the base sequence reads the same backwards and forwards. The mirror-like palindrome is similar to those found in ordinary text, in which a sequence reads the same forward and backward on a single-strand of DNA, e.g., GTAATG (SEQ ID NO: 240). The inverted repeat palindrome is also a sequence that reads the same forward and backward, but the forward and backward sequences are found in complementary DNA strands (i.e., of double-stranded DNA), e.g., GTATAC (SEQ ID NO: 241) being complementary to CATATG (SEQ ID NO: 242)). Inverted repeat palindromes are more common and have greater biological importance than mirror-like palindromes.

In one embodiment, a restriction site in the template is an uncommon restriction site, i.e., it is not commonly found in the sequence of transgenes. For example, the restriction site is a mirror-like palindrome restriction site, or an 8 base pair restriction site. In one embodiment, the restriction site used in the template is not found in the transgene, i.e., therapeutic transgene, of the invention. One skilled in the art can assess whether a particular restriction site is found within a particular transgene sequence, for example, by performing a nucleotide alignment of the restriction site and transgene sequences using Basic Local Alignment Search Tool (BLAST).

In one embodiment, the restriction site is selected from Table 1. When a restriction site is selected from Table 1, the corresponding restriction enzyme is used to cleave the restriction site.

TABLE 1
Known restriction sites and the corresponding restriction enzyme that cleaves the site.

| Restriction site | SEQ ID NO: | Restriction Enzyme that cleaves |
|---|---|---|
| A/AGCTT | 1 | HindIII HindIII-HF ® |
| AAT/ATT | 2 | SspI SspI-HF ® |
| /AATT | 3 | MluCI |
| A/CATGT | 4 | PciI |
| A/CCGGT | 5 | AgeI AgeI-HF ® |
| ACCTGC(4/8) | 6 | BfuAI BspMI |
| A/CCWGGT | 7 | SexAI |
| A/CGCGT | 8 | MluI MluI-HF ® |
| ACGGC(12/14) | 9 | BceAI |
| A/CGT | 10 | HpyCH4IV |
| ACN/GT | 11 | HpyCH4III |
| (10/15)ACNNNNGTAYC(12/7) | 12 | BaeI |
| (9/12)ACNNNNNCTCC(10/7) | 13 | BsaXI |
| A/CRYGT | 14 | AflIII |
| A/CTAGT | 15 | SpeI SpeI-HF ® |
| ACTGG(1/-1) | 16 | BsrI |
| ACTGGG(5/4) | 17 | BmrI |
| A/GATCT | 18 | BglII |
| AGC/GCT | 19 | AfeI |
| AG/CT | 20 | AluI |
| AGG/CCT | 21 | StuI |
| AGT/ACT | 22 | ScaI-HF ® |
| AT/CGAT | 23 | BspDI ClaI |
| ATCTATGTCGGGTGCGGAGAAAGAGGTAAT | 24 | PI-SceI |
| (-15/-19) | | |
| ATGCA/T | 25 | NsiI NsiI-HF ® |
| AT/TAAT | 26 | AseI |
| ATTT/AAAT | 27 | SwaI |
| (11/13)CAANNNNNGTGG(12/10) | 28 | CspCI |
| C/AATTG | 29 | MfeI-HF ® MfeI |
| CACGAG(-5/-1) | 30 | BssSI-v2 |
| CACGTC(-3/-3) | 31 | BmgBI |
| CAC/GTG | 32 | PmlI |
| CACNNN/GTG | 33 | DraIII-HF ® |
| CACNN/NNGTG | 34 | AleI-v2 |
| CAGCAG(25/27) | 35 | EcoP15I |
| CAG/CTG | 36 | Pvuii-HF ® Pvuii |
| CAGNNN/CTG | 37 | AlwNI |
| CAGTG(2/0) | 38 | BtsIMutI |
| CA/TATG | 39 | NdeI |
| C/ATG | 40 | CviAII |
| CATG/ | 41 | NlaIII |
| CAYNN/NNRTG | 42 | MslI |
| CC(12/16) | 43 | FspEI |
| CCANNNNN/NNNNTGG | 44 | XcmI |
| CCANNNNN/NTGG | 45 | BstXI |
| CCANNNN/NTGG | 46 | PflMI |
| CCATC(4/5) | 47 | BccI |
| C/CATGG | 48 | NcoI NcoI-HF ® |
| CCCAGC(-5/-1) | 49 | BseYI |
| CCCGC(4/6) | 50 | FauI |
| CCC/GGG | 51 | SmaI |
| C/CCGGG | 52 | TspMI XmaI |
| (0/-1)CCD | 53 | Nt.CviPII |
| CCDG(10/14) | 54 | LpnPI |
| CCGC(-3/-1) | 55 | AciI |
| CCGC/GG | 56 | SacII |
| CCGCTC(-3/-3) | 57 | BsrBI |
| C/CGG | 58 | HpaII MspI |
| CC/NGG | 59 | ScrFI |
| /CCNGG | 60 | StyD4I |
| C/CNNGG | 61 | BsaJI |
| CCNNNNN/NNGG | 62 | BslI |
| C/CRYGG | 63 | BtgI |
| CC/SGG | 64 | NciI |
| C/CTAGG | 65 | AvrII |
| CCTC(7/6) | 66 | MnlI |
| CCTCAGC(-5/-7) | 67 | Nt.BbvCI |
| CCTCAGC(-5/-2) | 68 | BbvCI |
| CCTGCA/GG | 69 | SbfI SbfI-HF ® |
| CCTNAGC(-5/-2) | 70 | Bpu10I |
| CC/TNAGG | 71 | Bsu36I |
| CCTNN/NNNAGG | 72 | EcoNI |
| CCTTC(6/5) | 73 | HpyAV |
| /CCWGG | 74 | PspGI |
| CC/WGG | 75 | BstNI |
| C/CWWGG | 76 | StyI StyI-HF ® |
| (10/12)CGANNNNNNTGC(12/10) | 77 | BcgI |
| CGAT/CG | 78 | PvuI PvuI-HF ® |
| CG/CG | 79 | BstUI |
| C/GGCCG | 80 | EagI EagI-HF ® |
| CG/GWCCG | 81 | RsrII |
| CGRY/CG | 82 | BsiEI |
| C/GTACG | 83 | BsiWI BsiWI-HF ® |
| CGTCTC(1/5) | 84 | BsmBI Esp3I |
| CGWCG/ | 85 | Hpy99I |
| CMG/CKG | 86 | MspA1I |
| CNNNNNNG | 87 | AbaSI |
| CNNR(9/13) | 88 | MspJI |
| CR/CCGGYG | 89 | SgrAI |
| C/TAG | 90 | BfaI |
| CTCAG(9/7) | 91 | BspCNI |
| C/TCGAG | 92 | PaeR7I XhoI |
| CTCTTC(1/4) | 93 | EarI |
| CTGAAG(16/14) | 94 | AcuI |
| CTGCA/G | 95 | PstI PstI-HF ® |
| CTGGAG(16/14) | 96 | BpmI |
| C/TNAG | 97 | DdeI |
| C/TRYAG | 98 | SfcI |
| C/TTAAG | 99 | AflII |
| CTTGAG(16/14) | 100 | BpuEI |
| C/TYRAG | 101 | SmlI |
| C/YCGRG | 102 | AvaI BsoBI |
| GAAGA(8/7) | 103 | MboII |
| GAAGAC(2/6) | 104 | BbsI-HF ® BbsI |
| GAANN/NNTTC | 105 | XmnI |
| GAATGC(1/-1) | 106 | BsmI |
| G/AATTC | 107 | EcoRI-HF ® EcoRI |
| GACGC(5/10) | 108 | HgaI |
| GACGT/C | 109 | AatII |
| GAC/GTC | 110 | ZraI |
| GACN/NNGTC | 111 | Tth111I PflFI |
| GACNN/NNGTC | 112 | PshAI |
| GACNNN/NNGTC | 113 | AhdI |
| GACNNNN/NNGTC | 114 | DrdI |
| GAGCT/C | 115 | SacI SacI-HF ® |
| GAG/CTC | 116 | Eco53kI |
| GAGGAG(10/8) | 117 | BseRI |
| GAGTC(4/-5) | 118 | Nt.BstNBI |
| GAGTC(4/5) | 119 | PleI |
| GAGTC(5/5) | 120 | MlyI |
| G/ANTC | 121 | HinfI |
| GAT/ATC | 122 | EcoRV EcoRV-HF ® |
| GA/TC | 123 | DpnI |
| /GATC | 124 | Sau3AI MboI DpnII |
| GATNN/NNATC | 125 | BsaBI |
| G/AWTC | 126 | TfiI |
| GCAATG(2/0) | 127 | BsrDI |
| GCAGC(8/12) | 128 | BbvI |
| GCAGTG(2/0) | 129 | BtsI-v2 |
| GCANNNN/NTGC | 130 | BstAPI |
| GCATC(5/9) | 131 | SfaNI |
| GCATG/C | 132 | SphI SphI-HF ® |
| GCCC/GGGC | 133 | SrfI |
| GCCGAG(21/19) | 134 | NmeAIII |
| G/CCGGC | 135 | NgoMIV |
| GCC/GGC | 136 | NaeI |
| GCCNNNN/NGGC | 137 | BglI |
| GCGAT/CGC | 138 | AsiSI |
| GCGATG(10/14) | 139 | BtgZI |
| G/CGC | 140 | HinP1I |
| GCG/C | 141 | HhaI |
| G/CGCGC | 142 | BssHII |
| GC/GGCCGC | 143 | NotI NotI-HF ® |
| GC/NGC | 144 | Fnu4HI |
| GCN/NGC | 145 | Cac8I |
| GCNNNNN/NNGC | 146 | MwoI |
| G/CTAGC | 147 | NheI NheI-HF ® |
| GCTAG/C | 148 | BmtI BmtI-HF ® |
| GCTCTTC(1/-7) | 149 | Nt.BspQI |
| GCTCTTC(1/4) | 150 | BspQI SapI |
| GC/TNAGC | 151 | BlpI |
| G/CWGC | 152 | ApeKI TseI |
| GDGCH/C | 153 | Bsp1286I |
| GGATC(4/5) | 154 | AlwI |
| GGATC(4/-5) | 155 | Nt.AlwI |
| G/GATCC | 156 | BamHI BamHI-HF ® |
| GGATG(2/0) | 157 | BtsCI |
| GGATG(9/13) | 158 | FokI |
| GG/CC | 159 | HaeIII |
| GGCCGG/CC | 160 | FseI |
| GGCCNNNN/NGGCC | 161 | SfiI |
| GGC/GCC | 162 | SfoI |
| G/GCGCC | 163 | KasI |
| GG/CGCC | 164 | NarI |
| GGCGC/C | 165 | PluTI |
| GG/CGCGCC | 166 | AscI |
| GGCGGA(11/9) | 167 | EciI |
| GGGAC(10/14) | 168 | BsmFI |
| GGGCC/C | 169 | ApaI |
| G/GGCCC | 170 | PspOMI |
| G/GNCC | 171 | Sau96I |
| GGN/NCC | 172 | NlaIV |
| G/GTACC | 173 | Acc65I |
| GGTAC/C | 174 | KpnI KpnI-HF ® |
| GGTCTC(1/5) | 175 | BsaI BsaI-HF ® v2 |
| GGTGA(8/7) | 176 | HphI |
| G/GTNACC | 177 | BstEII BstEII-HF ® |
| G/GWCC | 178 | AvaII |
| G/GYRCC | 179 | BanI |
| GKGCM/C | 180 | BacGI |
| GR/CGYC | 181 | BsaHI |
| GRGCY/C | 182 | BanII |
| GT/AC | 183 | RsaI |
| G/TAC | 184 | CviQI |
| GTATAC | 185 | BstZ17I-HF ® |
| GTATCC(6/5) | 186 | BciVI |
| G/TCGAC | 187 | SalI SalI-HF ® |
| GTCTC(1/5) | 188 | BcoDI BsmAI |
| GTCTC(1/-5) | 189 | Nt.BsmAI |
| G/TGCAC | 190 | ApaLI |
| GTGCAG(16/14) | 191 | BsgI |
| GT/MKAC | 192 | AccI |
| GTN/NAC | 193 | Hpy166II |
| /GTSAC | 194 | Tsp45I |
| GTT/AAC | 195 | HpaI |
| GTTT/AAAC | 196 | PmeI |
| GTY/RAC | 197 | HincII |
| GWGCW/C | 198 | BsiHKAI |
| NNCASTGNN/ | 199 | TspRI |
| R/AATTY | 200 | ApoI-HF ApoI |
| RCATG/Y | 201 | NspI |
| R/CCGGY | 202 | BsrFI-v2 |
| R/GATCY | 203 | BstYI |
| RGCGC/Y | 204 | HaeII |
| RG/CY | 205 | CviKI-1 |
| RG/GNCCY | 206 | EcoO109I |
| RG/GWCCY | 207 | PpuMI |
| TAACTATAACGGTCCTAAGGTAGCGAA | 208 | I-CeuI |
| (-9/-13) | | |
| TAC/GTA | 209 | SnaBI |
| TAGGGATAACAGGGTAAT(-9/-13) | 210 | I-SceI |
| T/CATGA | 211 | BspHI |
| T/CCGGA | 212 | BspEI |
| TCCRAC(20/18) | 213 | MmeI |
| T/CGA | 214 | TaqI-v2 |
| TCG/CGA | 215 | NruI NruI-HF ® |
| TCN/GA | 216 | Hpy188I |
| TC/NNGA | 217 | Hpy188III |
| T/CTAGA | 218 | XbaI |
| T/GATCA | 219 | Bcli BclI-HF |
| TG/CA | 220 | HpyCH4V |
| TGC/GCA | 221 | FspI |
| TGGCAAACAGCTATTATGGGTATTATGGGT | 222 | PI-PspI |
| (-13/-17) | | |
| TGG/CCA | 223 | MscI |
| T/GTACA | 224 | BsrGI BsrGI-HF ® |
| T/TAA | 225 | MseI |
| TTAAT/TAA | 226 | PacI |
| TTA/TAA | 227 | PsiI |
| TT/CGAA | 228 | BstBI |
| TTT/AAA | 229 | DraI |
| VC/TCGAGB | 230 | PspXI |
| W/CCGGW | 231 | BsaWI |
| YAC/GTR | 232 | BsaAI |
| Y/GGCCR | 233 | EaeI |

In Table 1, all restriction sites are written 5′ to 3′ using the single letter code nomenclature with the point of cleavage indicated by a “/”. Numbers in parentheses indicate point of cleavage for non-palindromic enzymes. For example, GGTCTC(1/5) indicates cleavage at: 5′ . . . GGTCTCN/ . . . 3′ and its complement, 3′ . . . CCAGAGNNNNN/ . . . 5′.

In one embodiment, the template comprises at least one SwaI restriction site, e.g., at least 1, at least 2, or more SwaI restriction sites. A SwaI restriction site has a octanucleotide sequence of 5′-ATTTAAAT-3′ (SEQ ID NO: 27). The SwaI restriction enzyme cleaves in the center of the restriction sequence, creating blunt ended DNA fragments.

In one embodiment, the at least two restriction sites are identical. For example, a template can contain two Sf1 restriction sites. Alternatively, the at least two restriction sites are different. For example, a template can have a Sfi1 restriction site and a MwoI restriction site. In one embodiment, at least two complementary SwaI sequences are annealed to from a loop within the template sequence. A SwaI loop can be cleaved with SwaI restriction enzyme.

Typically, to cut a cleavage site, the nucleic acid is contacted by an enzyme that activates the cleavage site, e.g., a protelomerase or a restriction enzyme, for an amount of time, and under the conditions, sufficient to cut the cleavage site. One skilled in the art can determine the correct conditions, e.g., temperature, concentration of reagent in reaction, and timing of the contact, for a given enzyme. For example, the correct conditions for known restriction enzymes can be found on the world wide web at www.enzymefinder.neb.com.

Adaptor Sequences

An adaptor sequence is a short, synthesized, single-stranded or double-stranded oligonucleotide that can be ligated to the ends of other DNA or RNA molecules. In one embodiment, an adaptor sequence described herein is single-stranded and close the DNA end it is ligated to, e.g., through a hairpin loop. Adaptor sequences are added to one or both ends of the cut plasmid fragment as a means circularizing the DNA. In one embodiment, the adaptor sequence is ligated to the plasmid fragment and directs the closure at the end of the cleaved DNA which it is ligated to (see e.g., FIG. 1-5, 7, or 9). Exemplary adaptor proteins that can be used to close the DNA end include hairpin loops further described, e.g., in U.S. Application No. 2009/0098612; and U.S. Pat. Nos 6,369,038; 6,451,563; 6,849,725; the contents of which are incorporated herein by reference in their entireties. It is envisioned that any sequence that can circularize the DNA when added to the cut end of a plasmid fragment excised from a plasmid can be an adaptor sequence.

In various embodiments, an adaptor sequence or a complementary adaptor sequence is ligated to the sticky ends of a plasmid fragment cut by a nuclease, e.g., a restriction enzyme. Adaptor sequences can be hybridized to any plasmid fragment via methods described herein. In one embodiment, the adaptor sequence further comprises a restriction site sequence that facilitates its ligation/hybridization to the plasmid fragment having the same or complementary restriction sites following its excision from the plasmid. One skilled in the art will know how to add a restriction site sequence to an adaptor sequence, for example, using standard sub-cloning methods or PCR-based techniques. In order to ligate an adaptor sequence having a restriction site sequence, one must cut the restriction site, e.g., via contacting the adaptor sequence with a corresponding restriction enzymes. Methods for ligating restriction sites and complementary restriction sites are well known in the art, and can be found, e.g., on the world wide web at www.neb.com. For example, an excised vector and an adaptor protein are incubated in vitro in the presence of a ligase, e.g., T4 ligase, and ATP

By way of example, a hairpin loop adaptor sequence having the sequence of SEQ ID NO. 243 can further comprise an Sfi1 restriction site sequence (e.g., SEQ ID NO: 161). The adaptor sequence having a Sfi1 restriction site sequence can be digested with the restriction enzyme, Sfi1, for a time sufficient to cut the restriction site. This would create “sticky ends” on the adaptor sequence that can be used to hybridize the adaptor protein to a plasmid fragment excised via the Sfi1 restriction enzyme.

SEQ ID NO: 243 is the nucleotide sequence of a hairpin loop adaptor protein.

(SEQ ID NO: 243)
ccattctgttccgcatgattcctctgcggaacagaatgg

Promoters

In one embodiment, the transgene is operatively linked to a promoter. Various promoters that direct expression of the transgene are described herein. Examples include, but are not limited to, constitutive promoters, repressible promoters, and/or inducible promoters, some non-limiting examples of which include viral promoters (e.g., CMV, SV40), tissue specific promoters (e.g., muscle MCK), heart (e.g., NSE), eye (e.g., MSK) and synthetic promoters (SP1 elements) and chicken beta actin promoter (CB or CBA). The promoter can be present in any position on where it is in operable association with the nuclease sequence.

Inducible Promoters

An inducible promoter may be a promoter induced by the presence of an inducer, the absence of a repressor, or any other suitable physical or chemical condition that induces transcription from the inducible promoter. The terms “inducer”, “inducing conditions” and suchlike should be understood accordingly.

By way of non-limiting example, an inducible promoter for use in embodiments of the invention may be a small molecule-inducible promoter, a tetracycline-regulatable (e.g. inducible or repressible) promoter, an alcohol-inducible promoter, a steroid-inducible promoter, a mifepristone (RU486)-inducible promoter, an ecdysone-inducible promoter, a rapamycin-inducible promoter, a metallothionein-inducible promoter, a hormone-inducible promoter, a cumate-inducible promoter, a temperature-inducible promoter, a pH-inducible promoter and a metal-inducible promoter.

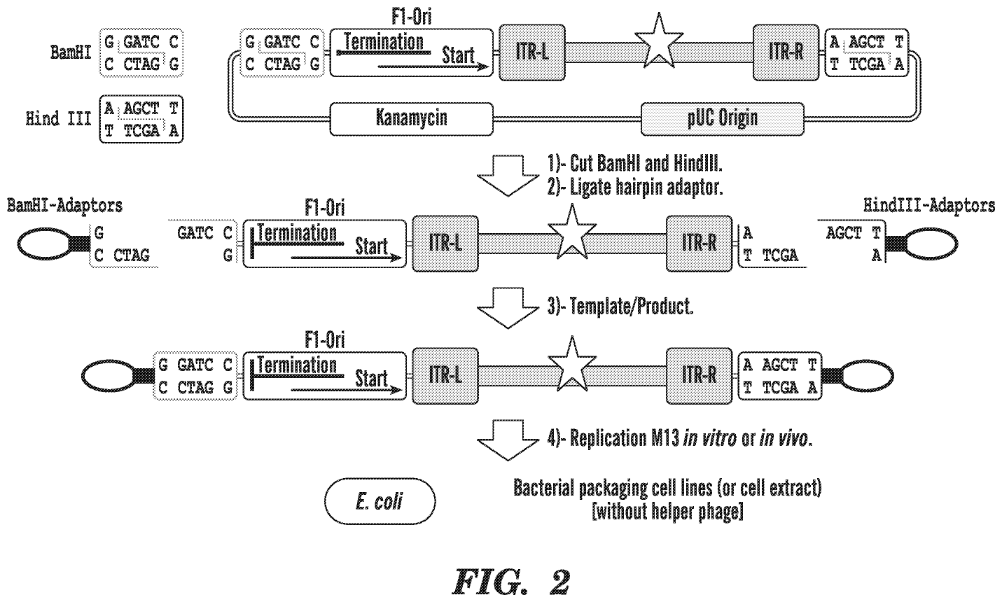

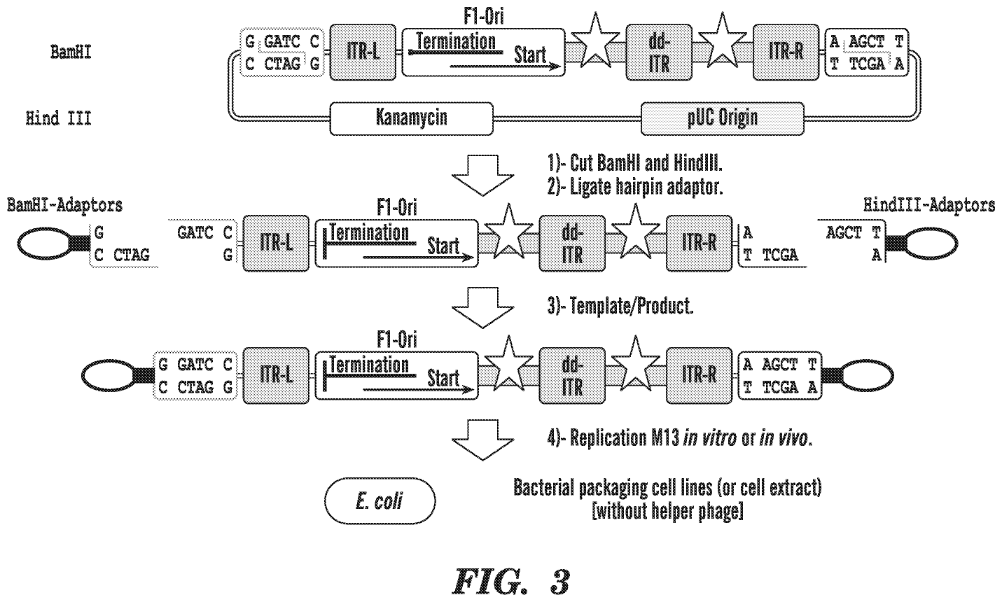

Temperature-Inducible Promoters—The inducible promoter may be induced by reduction of temperature, e.g. a cold-shock responsive promoter. In some embodiments, the inducible promoter is a synthetic cold-shock responsive promoter derived from the S1006a gene (calcyclin) of CHO cells. The temperature sensitivity of the S1006a gene (calcyclin) promoter was identified by Thaisuchat et al., 2011 (Thaisuchat, H. et al. (2011) ‘Identification of a novel temperature sensitive promoter in cho cells’, BMC Biotechnology, 11. doi: 10.1186/1472-6750-11-51), which is incorporated herein by reference. In some embodiments, the inducible promoter is one of the synthetic cold-shock responsive promoters shown in FIG. 2 of Thaisuchat et al., 2011. These promoters are induced by decrease of temperature as shown in FIG. 3 of Thaisuchat et al., 2011. Most of these synthetic promoter constructs show expression similar to the known promoter SV40 at 37° C. and are induced by 2-3 times when the temperature is reduced to 33° C. In some embodiments, the inducible promoter is sps5 from FIG. 2 of Thaisuchat et al., 2011. In some preferred embodiments, the inducible promoter is sps8 from FIG. 2 of Thaisuchat et al., 2011.

pH-Inducible Promoters—The inducible promoter may be induced by reduction or increase of pH to which cells comprising the promoter are exposed. Suitably, the inducible promoter may be induced by reduction of pH, i.e. a promoter inducible under acidic conditions. Suitable acid-inducible promoters are described in Hou et al., 2016 (Hou, J. et al. (2016) ‘Isolation and functional validation of salinity and osmotic stress inducible promoter from the maize type-II H+-pyrophosphatase gene by deletion analysis in transgenic tobacco plants’, PLoS ONE, 11(4), pp. 1-23. doi: 10.1371/journal.pone.0154041), which is incorporated herein by reference.

In some embodiments, the inducible promoter is a synthetic promoter inducible under acidic conditions derived from the YGP1 gene or the CCW14 gene. The inducibility by acidic conditions of the YGP1 gene or the CCW14 gene was studied and improved by modifying transcription factor binding sites by Rajkumar et al., 2016 (Rajkumar, A. S. et al. (2016) ‘Engineering of synthetic, stress-responsive yeast promoters’, 44(17). doi: 10.1093/nar/gkw553), which is incorporated herein by reference. In some embodiments, the inducible promoter is one of the synthetic promoter inducible under acidic conditions in FIGS. 1A, 2A, 3A and 4A of Rajkumar et al., 2016. These promoters are induced by decrease of pH as shown in FIGS. 1B, 2B, 3B and 4B of Rajkumar et al., 2016. Most of these synthetic promoters are induced by up to 10-15 times when the reduced from pH 6 to pH 3. In some preferred embodiments, the inducible promoter is YGP 1pr from FIG. 1 of Rajkumar et al., 2016. In other preferred embodiments, the inducible promoter is YGP 1pr from FIG. 1 of Rajkumar et al., 2016.

Osmolarity-Inducible Promoters—The inducible promoter may be osmolarity-induced. Suitable promoters induced by osmolarity are described in Zhang et al. (Molecular Biology Reports volume 39, pages7347-7353(2012)) which is incorporated herein by reference.

Carbon Source-Inducible Promoters—The inducible promoter may be induced by addition of a specific carbon source, e.g. a non-sugar carbon source. Alternatively, the inducible promoter may be induced by withdrawal or the absence of a carbon source. Suitable promoters induced by the presence or absence of various carbon sources are described in Weinhandl et al., 2014 (Weinhandl, K. et al. (2014) ‘Carbon source dependent promoters in yeasts’, Microbial Cell Factories, 13(1), pp. 1-17. doi: 10.1186/1475-2859-13-5), which is incorporated herein by reference.

Alcohol (e.g. Ethanol)-Inducible Promoters—The inducible promoter may be induced by addition of ethanol. Suitable promoters induced by ethanol are described in Matsuzawa et al. (Applied Microbiology and Biotechnology volume 97, pages6835-6843(2013)), which is incorporated herein by reference.

Amino Acid-Inducible Promoters—The inducible promoters may be induced by addition of one or more amino acids. Suitably, the amino acid may be an aromatic amino acid. Suitably, the amino acid may be GABA (gamma aminobutyric acid), which is also a neurotransmitter. Suitable promoter induced by aromatic amino acids and GABA are described in Kim et al. (Applied Microbiology and Biotechnology, volume 99, pages2705-2714(2015)) which is incorporated herein by reference.

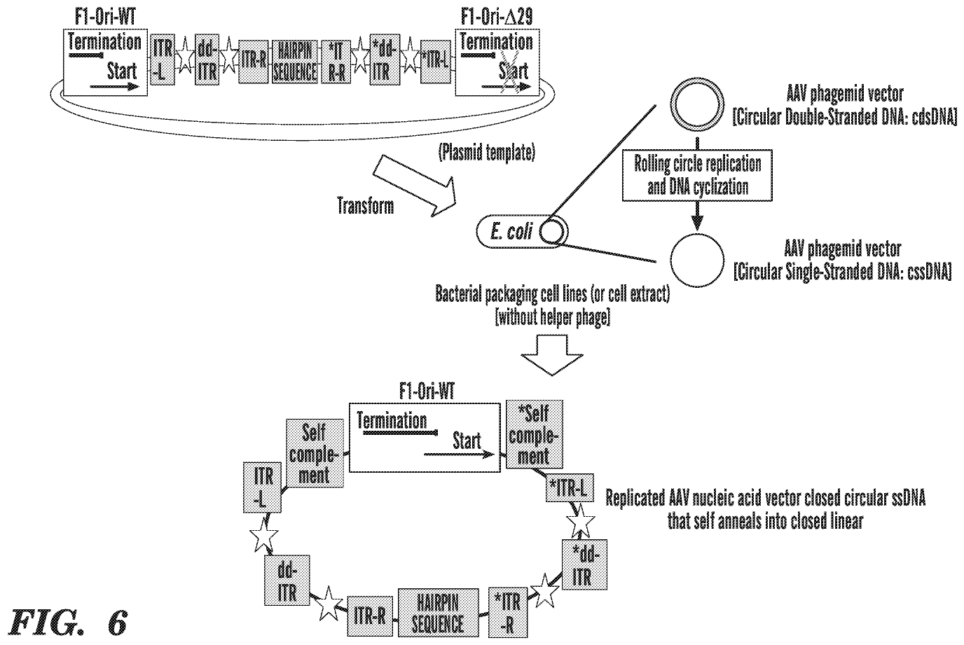

Hormone (e.g. Ecdysone)-Inducible Promoters—The inducible promoter may be the induced by a steroid hormone. Suitably, the steroid hormone may be ecdysone. A mammalian ecdysone-inducible system was created by No, Yao and Evans (No, D., Yao, T. P. and Evans, R. M. (1996) ‘Ecdysone-inducible gene expression in mammalian cells and transgenic mice’, Proceedings of the National Academy of Sciences of the United States of America, 93(8), pp. 3346-3351. doi: 10.1073/pnas.93.8.3346), which is incorporated herein by reference. Expression of a modified ecdysone receptor in mammalian cells allows expression from an ecdysone responsive promoter to be induced upon addition of ecdysone as shown in FIG. 2 of No, Yao and Evans, 1996. This system showed lower basal activity and higher inducibility than the tetracycline-inducible system as shown in FIG. 6 of No, Yao and Evans, 1996. A suitable commercially available inducible system is available from Agilent technologies and is described in Agilent Technologies (2015) ‘Complete Control Inducible Mammalian Expression System Instruction Manual’, 217460, which is incorporated herein by reference.

Tetracycline-Regulated Promoters—In some embodiments, the promoter may be induced by the presence or absence of tetracycline or its derivatives.

A suitable promoter induced in the absence of tetracycline or its derivatives is the promoter in the tet-OFF system. In the tet-OFF system, tetracycline-controlled transactivator (tTA) allows transcriptional activation of a tTA-dependent promoter in the absence of tetracycline or its derivatives. tTA and the tTA-dependent promoter were initially created by Gossen and Bujard, 1992 (Gossen, M. and Bujard, H. (1992) ‘Tight control of gene expression in mammalian cells by tetracycline-responsive promoters’, Proceedings of the National Academy of Sciences of the United States of America, 89(12), pp. 5547-5551. doi: 10.1073/pnas.89.12.5547), which is incorporated herein by reference. tTA was created by fusion of the tetracycline resistance operon (tet repressor) encoded in Tn10 of Escherichia coli with the activating cycline-controlled transactivator (tTA) and the tTA-dependent promoter was created by combining the tet operator sequence and a minimal promoter from the human cytomegalovirus promoter IE (hCMV-IE). When tetracycline or its derivatives are added, tTA can no longer bind its target sequence within the tTA-dependent promoter and there is no expression from the tTA-dependent promoter. This is shown in FIG. 1A and explained on page s96 of Jaisser, 2000 (Jaisser, F. (2000) ‘Inducible gene expression and gene modification in transgenic mice’, Journal of the American Society of Nephrology, 11(SUPPL. 16), pp. 95-100), which is incorporated herein by reference. The mechanism of the conformational change brought by binding of tetracycline or its derivatives to tTA is described in Orth et al., 2000 (Orth, P. et al. (2000) ‘Structural basis of gene regulation by the tetracycline inducible Tet repressor-operator system’, Nature Structural Biology, 7(3), pp. 215-219. doi: 10.1038/73324), which is incorporated herein by reference. Binding of tetracycline to TetR increases the separation of the attached DNA binding domains which abolishes the affinity of TetR for its operator DNA.

A suitable promoter induced by presence of tetracycline or its derivatives is the promoter in the tet-ON system. In the tet-ON system, a reverse tetracycline-controlled transactivator (rtTA) allows transcriptional activation of a tTA-dependent promoter in the presence of tetracycline or its derivatives as described in Gossen et al (Science 23 Jun. 1995: Vol. 268, Issue 5218, pp. 1766-1769 DOI: 10.1126/science.7792603), which is incorporated herein by reference. In the absence of tetracycline or its derivatives, tTA can no longer bind its target sequence within the tTA-dependent promoter and there is no expression from the tTA-dependent promoter. This is shown in FIG. 1B and explained on page s96 of Jaisser, 2000 (Jaisser, F. (2000) ‘Inducible gene expression and gene modification in transgenic mice’, Journal of the American Society of Nephrology, 11(SUPPL. 16), pp. 95-100), which is incorporated herein by reference.

Suitably, an improved variant of the reverse tetracycline-controlled transactivator (rtTA) is used.

Suitable improved variants are described in table 1 of Urlinger et al., 2000 (Urlinger, S. et al. (2000) ‘Exploring the sequence space for tetracycline-dependent transcriptional activators: Novel mutations yield expanded range and sensitivity’, Proceedings of the National Academy of Sciences of the United States of America, 97(14), pp. 7963-7968. doi: 10.1073/pnas.130192197), which is incorporated herein by reference. Variants rtTA-S2 and rtTA-M2 were shown to have lower basal activity in FIG. 3 of Urlinger et al., 2000, which indicates minimal background expression from the tTA-dependent promoter in the absence of tetracycline or its derivatives. Additionally rtTA-M2 showed an increased sensitivity towards tetracycline and its derivatives as shown in in FIG. 3 of Urlinger et al., 2000 and functions at 10 fold lower concentrations than rtTA. In some preferred embodiments, the improved variant of rtTA is rtTA-M2 from of Urlinger et al., 2000.

Alternative improved variants are described in Table 1 of Zhou et al., 2006 (Zhou, X. et al. (2006) ‘Optimization of the Tet-On system for regulated gene expression through viral evolution’, Gene Therapy, 13(19), pp. 1382-1390. doi: 10.1038/sj.gt.3302780), which is incorporated herein by reference. The majority of these variants were shown to have higher transcriptional activity and doxycycline sensitivity than rtTA as described in FIG. 3 of Zhou et al., 2006. The highest performing variants were seven-fold more active and 100 times more sensitive to doxycycline. In some preferred embodiments, the improved variant of rtTA is V14, V15 or V16 from Zhou et al., 2006.

Suitable commercially available tetracycline-inducible system is the T-Rex system from Life-Technologies (see e.g. Life-Technologies (2014) ‘Inducible Protein Expression Using the T-REx™ System’, 1, pp. 1-12. Available at: www.lifetechnologies.com/de/de/home/references/protocols/proteins-expression-isolation-and-analysis/protein-expression-protocol/inducible-protein-expression-using-the-trex- system.reg.us.html/).

Induction via, e.g. Tetracycline Absence and Estrogen Presence—The inducible promoter may be induced by absence of a molecule and presence of a different molecule. In some embodiments, the inducible promoter may be induced by removal of tetracycline and addition of estrogen as described in Iida et al., 1996 (Iida, A. et al. (1996) ‘Inducible gene expression by retrovirus-mediated transfer of a modified tetracycline-regulated system.’, Journal of virology, 70(9), pp. 6054-6059. doi: 10.1128/jvi.70.9.6054-6059.1996), which is incorporated herein by reference. This specific inducibility was achieved by the addition of the ligand-binding domain of the estrogen receptor to the carboxy terminal of the tTA transactivator. Such modified transactivator was shown result in high expression of the gene of interest in the absence of tetracycline and the presence of estrogen as shown in FIG. 3 of Iida et al., 1996.

Induction via Small Molecule Enhancers—The inducible promoter may be induced by small molecule enhancers. Suitable promoters induced by small molecule enhancers such as aromatic carboxylic acids, hydroxamic acids and acetamides are described in Allen et al. (Biotechnol. Bioeng. 2008;100: 1193-1204), which is incorporated herein by reference.

Mifepristone (RU-486)-Inducible Promoters—The inducible promoter may be induced by a synthetic steroid. In some embodiments, the inducible promoter may be induced by mifepristone, also known as RU-486. A hybrid mifespristone-responsive transcription factor, LexPR transactivator, was created by Emelyanov and Parinov, 2008 (Emelyanov, A. and Parinov, S. (2008) ‘Mifepristone-inducible LexPR system to drive and control gene expression in transgenic zebrafish’, Developmental Biology, 320(1), pp. 113-121. doi: 10.1016/j.ydbio.2008.04.042, which is incorporated herein by reference) by fusion of the DNA-binding domain of the bacterial LexA repressor, a truncated ligand-binding domain of the human progesterone receptor and the activation domain of the human NF-kB/p65 protein. Upon addition of mifepristone, LexPR induces expression from a promoter sequence harbouring LexA binding sites as shown in FIG. 1 and FIG. 2 of Emelyanov and Parinov, 2008. Suitable commercially available mifepristone-inducible system is the GeneSwitch System (see e.g., Fisher, T. (1994) ‘Inducible Protein Expression Using GeneSwitch™ Technology’, pp. 1-25).

Cumate-Inducible Promoters—In some embodiments, the inducible promoter may be induced by the presence or the absence of cumate.

In the cumate switch system from Mullick et al., 2006 (Mullick, A. et al. (2006) ‘The cumate gene-switch: A system for regulated expression in mammalian cells’, BMC Biotechnology, 6, pp. 1-18. doi: 10.1186/1472-6750-6-43, which is incorporated herein by reference), a repressor CymR blocks transcription from a promoter comprising CuO sequence placed downstream of the promoter. Once cumate is added, the CymR repressor is unable to bind to CuO and transcription from a promoter comprising CuO can proceed. This is shown in FIG. 1B and FIG. 2 from Mullick et al., 2006.

In an alternative cumate switch system, a chimeric transactivator (cTA) created from the fusion of CymR with the activation domain of VP16 does not prevent transcription from a promoter comprising CuO sequence upstream of a promoter in the presence of cumate. In the absence of cumate, the chimeric transactivator (cTA) binds to the CuO sequence and prevents transcription. This is shown in FIG. 1C and FIG. 3 from Mullick et al., 2006.

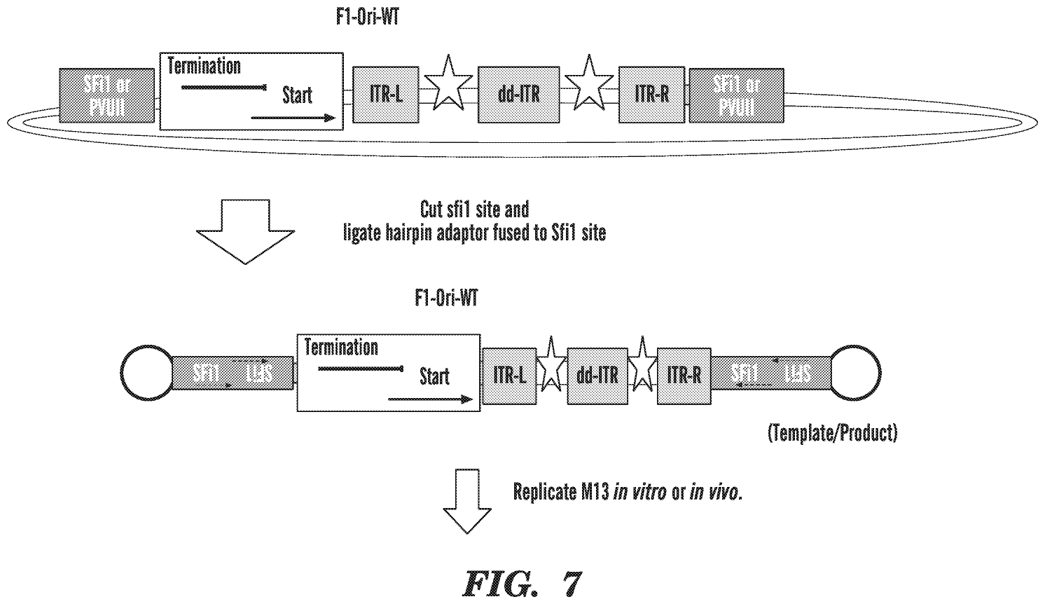

In a third configuration, a reverse chimeric transactivator (rcTA) prevents transcription from a promoter comprising CuO sequence upstream of a promoter in the absence of cumate. In the presence of cumate, the rcTA binds to the CuO sequence and transcription from the promoter comprising CuO sequence can proceed. This is shown in FIG. 1D and FIG. 7 from Mullick et al., 2006.

Suitable commercially available cumate-inducible systems is found from SBI Biosciences (see SBI (2020) ‘Cumate-inducible Systems For the ultimate in gene expression control , use SBI's cumate- CUMATE-INDUCIBLE SYSTEMS’, pp. 1-13, which is incorporated herein by reference).

4-hydroxytamoxifen (OHT)-Inducible Promoters—The inducible promoter may be induced by 4-hydroxytamoxifen (OHT). Suitable 4-hydroxytamoxifen inducible promoters are described by Feil et al. (Biochemical and Biophysical Research Communications Volume 237, Issue 3, 28 Aug. 1997, Pages 752), which is incorporated herein by reference.

Gas-Inducible Promoters—The inducible promoter may be a gas-inducible promoter, e.g. acetaldehyde-inducible. Suitable gas-inducible promoters are described in Weber et al., 2004 (Weber, W. et al. (2004) ‘Gas-inducible transgene expression in mammalian cells and mice’, Nature Biotechnology, 22(11), pp. 1440-1444. doi: 10.1038/nbt1021), which is incorporated herein by reference. The native acetaldehyde-inducible AlcR-PalcA system from Asperigillus nidulans has been adapted for mammalian use by introducing an AlcR-specific operator module to a human minimal promoter, together called P_{AIR}, as shown in FIG. 1A. When AlcR is constitutively expressed in the cell of interest, upon introduction of acetaldehyde, acetaldehyde binds to AlcR and, in turn, the gene of interest which is under the control of the PAIR promoter is expressed, as shown in FIG. 1C, FIG. 2 and FIG. 3. In the absence of acetaldehyde, there is no expression of the gene of interest.

Riboswitch, Ribozyme and Aptazyme-Inducible Promoters—The inducible promoter may be induced by the presence or absence of a ribozyme. The ribozyme can, in turn be, be induced by a ligand.

The inducible promoter may be induced in the absence of a metabolite. In some embodiments, the metabolite may be glucosamine-6-phosphate-responsive. Suitable ribozyme which acts as a glucosamine-6-phosphate-responsive gene repressor is described by Winkler et al., 2004 (Winkler, W. C. et al. (2004) ‘Control of gene expression by a natural metabolite-responsive ribozyme’, Nature, 428(6980), pp. 281-286. doi: 10.1038/nature02362), which is incorporated herein by reference. The ribozyme is activated by glucosamine-6-phosphate in a concentration dependent manner as shown in FIG. 2C and cleaves the messenger RNA of the glmS gene. Upon modification, it is possible that this natural system may be applied to control of a gene of interest other than the glmS gene.

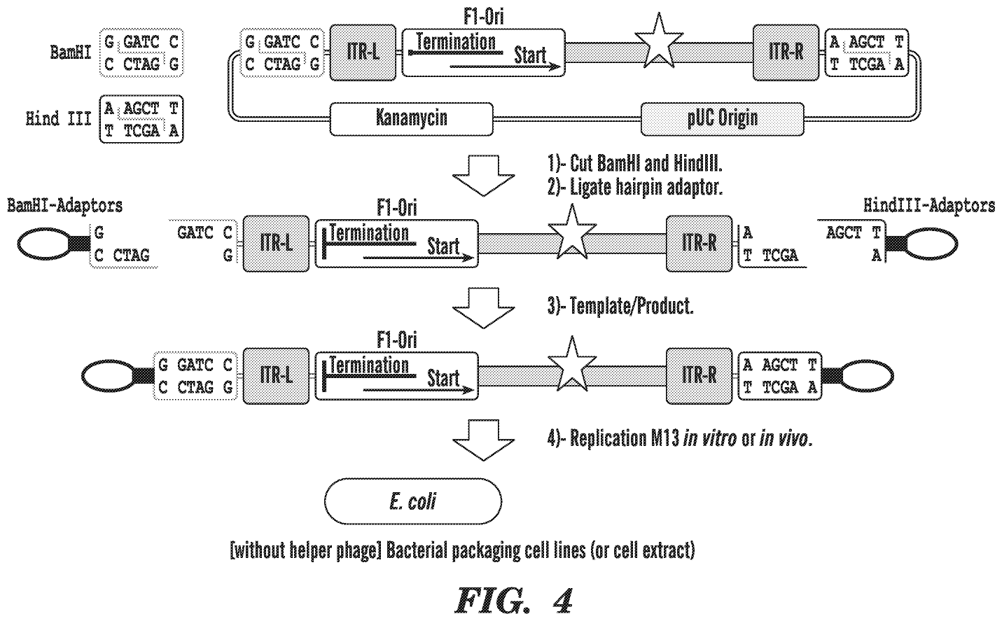

Protein expression can also be downregulated by ligand-inducible aptazyme. Protein expression can be downregulated by aptazyme which downregulate protein expression by small molecule-induced self-cleavage of the ribozyme resulting in mRNA degradation Zhong et al., 2016 (Zhong, G. et al. (2016) ‘Rational design of aptazyme riboswitches for efficient control of gene expression in mammalian cells’, eLife, 5(NOVEMBER 2016). doi: 10.7554/eLife.18858), which is incorporated herein by reference. Suitable aptazymes are shown in FIG. 4A of (Zhong et al., 2016). These aptazymes reduce relative expression of a gene of interest as shown in FIG. 4 of (Zhong et al., 2016).

Protein expression can also be upregulated by a small-molecule dependent ribozyme. The ribozyme may be tetracycline-dependent. Suitable tetracycline-dependent ribozymes which can switch on protein expression by preventing ribozyme cleavage which otherwise cleaves mRNA in the absence of ligand is described in Beilstein et al. (ACS Synth. Biol. 2015, 4, 5, 526-534), which is incorporated herein by reference.

Protein expression can also be regulated by a guanine dependent aptazyme as described by Nomura et al. (Chem. Commun., 2012,48, 7215-7217) which is incorporated herein by reference.

Additionally, an RNA architecture that combines a drug-inducible allosteric ribozyme with a microRNA precursor analogue that allows chemical induction of RNAi in mammalian cells is described in Kumar et al (J. Am. Chem. Soc. 2009, 131, 39, 13906-13907), which is incorporated herein by reference.

Metallothionein Inducible Promoters—Metallothionein-inducible promoters have been described in the literature. See for example Shinichiro Takahashi “Positive and negative regulators of the metallothionein gene” Molecular Medicine Reports Mar. 9, 2015, P795-799, which is incorporated herein by reference.

Rapamycin-Inducible Promoters—The inducible promoter may be induced by a small molecule drug such as rapamycin. A humanized system for pharmacologic control of gene expression using rapamycin is described in Rivera et al., 1996 (Rivera et al Nature Medicine volume 2, pages1028-1032(1996)), which is incorporated herein by reference. The natural ability of rapamycin to bind to FKBP12 and, in turn, for this complex to bind to FRAP was used by Rivera et al., 1996 to induce rapamycin-specific expression of a gene of interest. This was achieved by fusing one of the FKBP12/FRAP proteins to a DNA binding domain and the other protein to an activator domain. If the FKBP is fused with a DNA binding domain and FRAP is fused to an activator domain, there would be no transcription of the gene of interest in the absence of rapamycin since FKBP and FRAP do not interact, as shown in FIG. 1b. In the presence of rapamycin, FKBP and FRAP interact and the DNA binding domain and the activator domain are brought into close contact, resulting in transcription of the gene of interest as shown in FIG. 2 and FIG. 3.

Chemically Induced Proximity Inducible Promoters—The inducible promoter may be controlled by the chemically induced proximity. Suitable small molecule-based systems for controlling protein abundance or activities is described in Liang et al. (Sci Signal. 2011 Mar 15;4(164):rs2. doi: 10.1126/scisignal.2001449), which is incorporated herein by reference.

Gene expression may be induced by chemically induced proximity by a molecule combining two protein binding surfaces as shown in Belshaw et al., 1996 (Belshaw, P. J. et al. (1996) ‘Controlling protein association and subcellular localization with a synthetic ligand that induces heterodimerization of proteins’, Proceedings of the National Academy of Sciences of the United States of America, 93(10), pp. 4604-4607), which is incorporated herein by reference. Transcriptional activation of a gene of interest by chemically induced proximity by a molecule combining two protein binding surfaces is shown in FIG. 3 of Belshaw et al.

Rheoswitch® Inducible Promoters—The inducible promoter may be induced by small synthetic molecules. In some embodiments, these small synthetic molecules may be diacylhydrazine ligands. Suitable systems for inducible up- and down-regulation of gene expression is described in Cress et al. (Volume 66, Issue 8 Supplement, pp. 27) or Barrett et al. (Cancer Gene Therapy volume 25, pages106-116(2018)), which are incorporated herein by reference. The RheoSwitch® system consists of two chimeric proteins derived from the ecdysone receptor (EcR) and RXR that are fused to a DNA-binding domain and an acidic transcriptional activation domain, respectively. The nuclear receptors can heterodimerize to create a functional transcription factor upon binding of a small molecule synthetic ligand and activate transcription from a responsive promoter linked to a gene of interest.

CRISPR-Inducible Promoters—Gene expression may be induced by a on CRISPR-based transcription regulators. A nuclease-deficient Cas9 can be directed to a sequence of interest by designing its associated single guide RNA (sgRNA) and it can modulate the gene expression by tethering of effector domains on the sgRNA-Cas9 complex as shown in FIG. 1A of Ferry, Lyutova and Fulga, 2017 (Ferry, Q. R. V., Lyutova, R. and Fulga, T. A. (2017) ‘Rational design of inducible CRISPR guide RNAs for de novo assembly of transcriptional programs’, Nature Communications. Nature Publishing Group, 8, pp. 1-10. doi: 10.1038/ncomms14633), which is incorporated herein by reference. Suitable versatile inducible-CRISPR-TR platform based on minimal engineering of the sgRNA is described in Ferry, Lyutova and Fulga, 2017.

The CRISPR-based transcriptional regulation may in turn be induced by drugs. Suitable drug inducible CRISPR-based transcription regulators systems are shown in Zhang et al., 2019 (Zhang, J. et al. (2019) ‘Drug Inducible CRISPR/Cas Systems’, Computational and Structural Biotechnology Journal. Elsevier B.V., 17, pp. 1171-1177. doi: 10.1016/j.csbj.2019.07.015), which is incorporated herein by reference.

In one embodiment, contacting the cell with an inducer or applying a suitable inducing condition to the cell results in expression of the gene operatively linked to the inducible promoter.

Inducible promoters described herein can further control expression of an inducer or repressor of an inducible promoter, e.g., an inducer or repressor of a second, different promoter, or an inducer or repressor of itself. In one embodiment, the cell comprises a first inducible promoter that is operatively linked to a repressible element that can stop protein expression.

In one embodiment, the first inducible promoter that further encodes a protein that represses expression of the first inducible promoter.

In one embodiment, the cell comprises a first inducible promoter that further encodes a protein that induces expression of a second inducible promoter.

Tissue-Specific Promoters

In some embodiments of the methods and compositions as disclosed herein, the promoter is a liver specific promoter, and can be selected from promoters including, but not limited to, those disclosed in Table 2. Liver-specific” or “liver-specific expression” refers to the ability of a cis-regulatory element, cis-regulatory module or promoter to enhance or drive expression of a gene in the liver (or in liver-derived cells) in a preferential or predominant manner as compared to other tissues (e.g. spleen, muscle, heart, lung, and brain). Expression of the gene can be in the form of mRNA or protein. In preferred embodiments, liver-specific expression is such that there is negligible expression in other (i.e. non-liver) tissues or cells, i.e. expression is highly liver-specific.

A liver-specific promoter includes a liver-specific cis-regulatory element (CRE), a synthetic liver-specific cis-regulatory module (CRM) or a synthetic liver-specific promoter as disclosed herein, in Table 2. These liver-specific promoter elements include minimal liver-specific promoters.

Liver-specific promoter elements are further described in, e.g., International Application No. PCT/GB2019/053267, which is incorporated herein by reference in its entirety.

Table 2 shows exemplary liver-specific promoter sequences. The relatively small size of liver-specific promote sequence in Table 2 is advantageous because it takes up the minimal amount of the payload of the vector. This is particularly important when a CRE is used in a vector with limited capacity, such as an AAV-based vector.

TABLE 2
Liver-specific promoters (These are liver-specific promoters comprising cis-regulatory
modules (CRMs)):

| Liver-specific | |
|---|---|
| CRM Promoter | SEQUENCE |
| CRM_SP0131 | GGCCCGGGAGGCGCCCTTTGGACCTTTTGCAATCCTGGCGCACTGAACCCTTGACCCCTGCCCTG |
| (CRM_LVR_131) | CAGCCCCCGCAGCTTGCTGTTTGCCCACTCTATTTGCCCAGCCCCAGCCCTGGAGAGTCCTTTAG |
| | CAGGGCAAAGTGCAACATAGGCAGACCTTAAGGGATGACTCAGTAACAGATAAGCTTTGTGTGCC |
| | TGCA (SEQ ID NO: 244) |
| CRM_SP0239 | CAGGCTTTCACTTTCTCGCCAACTTACAAGGCCTTTCTGTGTAAACAATACCTGAACCTTTACCC |
| | CGTTGCCCGGCAACGGCCAGGTCTGTGCCAAGTGTTTGAGGTTAATTTTTAAAAAGCAGTCAAAA |
| | GTCCAAGTGGCCCTTGGCAGCATTTACTCTCTCTGTTTGCTCTGGTTAATAATCTCAGGAGCACA |
| | AACATTCCGGCCCGGGAGGCGCCCTTTGGACCTTTTGCAATCCTGGCGCACTGAACCCTTGACCC |
| | CTGCCCTGCAGCCCCCGCAGCTTGCTGTTTGCCCACTCTATTTGCCCAGCCCCAGCCCTGGAGAG |
| | TCCTTTAGCAGGGCAAAGTGCAACATAGGCAGACCTTAAGGGATGACTCAGTAACAGATAAGCTT |
| | TGTGTGCCTGCA (SEQ ID NO: 245) |
| CRM_SP0240 | CAGGCTTTCACTTTCTCGCCAACTTACAAGGCCTTTCTGTGTAAACAATACCTGAACCTTTACCC |
| | CGTTGCCCGGCAACGGCCAGGTCTGTGCCAAGTGTTTG (SEQ ID NO: 266) |
| CRM_SP0246 | AGGTTAATTTTTAAAAAGCAGTCAAAAGTCCAAGTGGCCCTTGGCAGCATTTACTCTCTCTGTTT |
| | GCTCTGGTTAATAATCTCAGGAGCACAAACATTCCGGCCCGGGAGGCGCCCTTTGGACCTTTTGC |
| | AATCCTGGCGCACTGAACCCTTGACCCCTGCCCTGCAGCCCCCGCAGCTTGCTGTTTGCCCACTC |
| | TATTTGCCCAGCCCCAG (SEQ ID NO: 246) |
| CRM_SP0265 | AGGTTAATTTTTAAAAAGCAGTCAAAAGTCCAAGTGGCCCTTGGCAGCATTTACTCTCTCTGTTT |
| (CRM_LVR_131_ | GCTCTGGTTAATAATCTCAGGAGCACAAACATTCCTGTACCGGCCCGGGAGGCGCCCTTTGGACC |
| A1) | TTTTGCAATCCTGGCGCACTGAACCCTTGACCCCTGCCCTGCAGCCCCCGCAGCTTGCTGTTTGC |
| | CCACTCTATTTGCCCAGCCCCAGCCCTGGAGAGTCCTTTAGCAGGGCAAAGTGCAACATAGGCAG |
| | ACCTTAAGGGATGACTCAGTAACAGATAAGCTTTGTGTGCCTGCA (SEQ ID NO: 286) |
| CRM_SP0412 | AGGTTAATTTTTAAAAAGCAGTCAAAAGTCCAAGTGGCCCTTGGCAGCATTTACTCTCTCTGTTT |
| | GCTCTGGTTAATAATCTCAGGAGCACAAACATTCCCCCTGTTCAAACATGTCCTAATACTCTGTC |
| | TCTGCAAGGGTCATCAGTAGTTTTCCATCTTACTCAACATCCTCCCAGTG (SEQ ID NO: |
| | 247) |

Other liver specific promoters include, but are not limited to promoters for the LDL receptor, Factor VIII, Factor IX, phenylalanine hydroxylase (PAH), ornithine transcarbamylase (OTC), and al-antitrypsin (hAAT), and HCB promoter. Other liver specific promoters include the AFP (alpha fetal protein) gene promoter and the albumin gene promoter, as disclosed in EP Patent Publication 0 415 731, the α-1 antitrypsin gene promoter, as disclosed in Rettenger, Proc. Natl. Acad. Sci. 91 (1994) 1460-1464, the fibrinogen gene promoter, the APO-Al (Apolipoprotein Al) gene promoter, and the promoter genes for liver transference enzymes such as, for example, SGOT, SGPT and y-glutamyle transferase. See also 2001/0051611 and PCT Patent Publications WO 90/07936 and WO 91/02805, which are incorporated herein in their entirety by reference. In some embodiments, the liver specific promoter is a recombinant liver specific promoter, e.g., as disclosed in US20170326256A1, which is incorporated herein in its entirety by reference.

In some embodiments, a liver specific promoter is the hepatitis B X-gene promoter and the hepatitis B core protein promoter. In some embodiments, liver specific promoters can be used with their respective enhancers. The enhancer element can be linked at either the 5′ or the 3′ end of the nucleic acid encoding the lysosomal enzyme. The hepatitis B X gene promoter and its enhancer can be obtained from the viral genome as a 332 base pair EcoRV-NcoI DNA fragment employing the methods described in Twu, J Virol. 61 (1987) 3448-3453. The hepatitis B core protein promoter can be obtained from the viral genome as a 584 base pair BamHI-BgIII DNA fragment employing the methods described in Gerlach, Virol 189 (1992) 59-66. It may be necessary to remove the negative regulatory sequence in the BamHI-BgIII fragment prior to inserting it.

Functional Variants of Liver-Specific Promoters

In some embodiments, a functional variant of a liver-specific promoter can be viewed as a promoter element which, when substituted in place of a reference promoter element in a promoter, substantially retains its activity. For example, a functional variant of liver-specific promoter which comprises a functional variant of a given promoter disclosed in Table 2 preferably retains at least 35%, or at least 40%, or at least 45%, or at least 50%, or at least 55%, or at least 60%, or at least 70% or at least 80% of its activity, more preferably at least 90% of its activity, more preferably at least 95% of the activity of the unchanged promoter, and yet more preferably 100% of the activity (as compared to the unchanged promoter sequence comprising the unmodified promoter element).

In some embodiments, a functional variant or a functional fragment of a liver-specific promoter disclosed in Table 2 has at least about 75% sequence identity to, or at least about 80% sequence identity to, at least about 90% sequence identity to, at least about 95% sequence identity to, at least about 98% sequence identity to the original unmodified sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence. Liver-specificity can be identified wherein the expression of a gene (e.g. a therapeutic or reporter gene) operatively linked to the promoter occurs preferentially or predominantly in liver-derived cells. Preferential or predominant expression can be defined, for example, where the level of expression is significantly greater in liver-derived cells than in other types of cells (i.e. non-liver-derived cells).

For example, a functional variant or a functional fragment of SEQ ID NO: 247 has at least about 75% sequence identity to SEQ ID NO: 247, or at least about 80% sequence identity to SEQ ID NO: 247, at least about 90% sequence identity to SEQ ID NO: 247, at least about 95% sequence identity to SEQ ID NO: 247, at least about 98% sequence identity to SEQ ID NO: 247, or the original unmodified sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence of SEQ ID NO: 247.

Suitably, functional variants of a promoter element retain a significant level of sequence identity to a reference promoter element. Suitably functional variants comprise a sequence that is at least 70% identical to the reference promoter element, more preferably at least 80%, 90%, 95% or 99% identical to the reference promoter element.

It should be noted that the sequences of a liver-specific promoter as disclosed herein in Table 2 can be altered without causing a substantial loss of activity. Thus, functional variants of a liver-specific promoter are discussed below can be prepared by modifying the sequence of a liver-specific promoter disclosed in Table 2, provided that modifications which are significantly detrimental to activity of the liver-specific promoter are avoided. In view of the information provided in the present disclosure, modification of a liver-specific promoter disclosed herein in Table 2 to provide functional variants is straightforward. Moreover, the present disclosure provides methodologies for simply assessing the functionality of any given liver-specific promoter variant.

Transgenes

The circular nucleic acids or vectors manufactured using methods of the present invention are useful for the delivery of nucleic acids to cells in vitro, ex vivo, and in vivo. In particular, the circular nucleic acids or vectors can be advantageously employed to deliver or transfer nucleic acids to animal, including mammalian, cells.

Any nucleic acid sequence(s) of interest may be delivered in the DNA constructs manufactured using the present invention. In one embodiment, nucleic acids of interest include nucleic acids encoding polypeptides, including viral polypeptides (e.g., any polypeptide expressed by a virus and/or required for viral particle production, such as Cap, Rep, Ad helper polypeptides, and the like), therapeutic polypeptides (e.g., for medical or veterinary uses), immunogenic polypeptides (e.g., for vaccines), or diagnostic polypeptides. In one embodiment, nucleic acids of interest include those nucleic acids encoding gene editing polypeptides, for example, CRISPR, Cas, TALENs, Meganucleases, or the like. In one embodiment, nucleic acids of interest include RNA interference nucleic acids, for example, a miRNA, a shRNA, an siRNA, a dsRNA, inhibitory oligonucleotides, or the like.

In one embodiment, the transgene is a therapeutic gene. Therapeutic genes include, but are not limited to, cystic fibrosis transmembrane regulator protein (CFTR), dystrophin (including mini-and micro-dystrophins (see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, beta-globin, alpha-globin, spectrin, alphas-antitrypsin, adenosine deaminase, hypoxanthine guanine phosphoribosyl transferase, beta-glucocerebrosidase, sphingomyelinase, lysosomal hexosaminidase A, branched-chain keto acid dehydrogenase, RP65 protein, cytokines (e.g., alpha-interferon, beta-interferon, interferon-gamma., interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor -3 and -4, brain-derived neurotrophic factor, bone morphogenic proteins [including RANKL and VEGF], glial derived growth factor, transforming growth factor -.alpha. and -.beta., and the like), lysosomal acid .alpha.-glucosidase, .alpha.-galactosidase A, receptors (e.g., the tumor necrosis growth factory soluble receptor), S100A1, parvalbumin, adenylyl cyclase type 6, a molecule that effects G-protein coupled receptor kinase type 2 knockdown such as a truncated constitutively active bARKet, anti-inflammatory factors such as TRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

Nucleic acid sequences encoding polypeptides include those encoding reporter polypeptides (e.g., an enzyme). Reporter polypeptides are known in the art and include, but are not limited to, Green Fluorescent Protein, beta-galactosidase, alkaline phosphatase, luciferase, and chloramphenicol acetyltransferase gene.

Alternatively, in particular embodiments of this invention, the nucleic acid, e.g., transgene, may encode an antisense nucleic acid, a ribozyme (e.g., as described in U.S. Pat. No. 5,877,022), RNAs that effect spliceosome-mediated trans-splicing (see, Puttaraju et al., (1999) Nature Biotech 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan (e.g., alpha., beta., gamma), RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the therapeutic transgene may encode protein phosphatase inhibitor I (I-1), serca2a, zinc finger proteins that regulate the phospholamban gene, Barkct, beta 2-adrenergic receptor, beta 2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenic proteins (including BNP 2, 7, etc., RANKL and/or VEGF).

The circular nucleic acids or vectors may also comprise a nucleic acid that shares homology with and recombines with a locus on a host chromosome. This approach can be utilized, for example, to correct a genetic defect in the host cell.

As a further alternative, the circular nucleic acids or vectors can encode any polypeptide that is desirably produced in a cell in vitro, ex vivo, or in vivo. For example, the circular nucleic acids or vectors may be introduced into cultured cells and the expressed gene product isolated therefrom.

In one embodiment, the therapeutic gene is operatively linked to a promoter. Various promoters that direct expression of the therapeutic transgene are described herein. Examples include, but are not limited to, constitutive promoters, repressible promoters, and/or inducible promoters, some non-limiting examples of which include viral promoters (e.g., CMV, SV40), tissue specific promoters (e.g., muscle MCK), heart (e.g., NSE), eye (e.g., MSK) and synthetic promoters (SP1 elements) and the chicken beta actin promoter (CB or CBA). The promoter can be present in any position on where it is in operable association with the nuclease sequence.

In addition, one or more promoters, which can be the same or different, can be present in the same nucleic acid molecule, either together or positioned at different locations on the nucleic acid molecule. Furthermore, an internal ribosome entry signal (IRES) and/or other ribosome-readthrough element can be present on the nucleic acid molecule. One or more such IRESs and/or ribosome readthrough elements, which can be the same or different, can be present in the same nucleic acid molecule, either together and/or at different locations on the nucleic acid molecule. Such IRESs and ribosome readthrough elements can be used to translate messenger RNA sequences via cap-independent mechanisms when multiple nuclease sequences are present on a nucleic acid molecule.

The circular nucleic acid vector of the present invention provides a means for enhanced transduction efficiency of nucleic acids into a broad range of cells, including a dividing cell, a non-dividing cells, a liver cell, a kidney cell, a CNS cell, a skin cell, a retinal cell, a cardiac cell, or the like, as compared to a standard DNA vector. In some embodiments, the transduction efficiency of a circular nucleic acid vector described herein is increased at least about 10% relative to a standard DNA vector, e.g., at least about 10%, 20%, 30%, 40%, 50%, 60%, 70%, 80%, 90%, 100%, 150%, 200%, 500%, or more, in comparison to plasmid vector or non-closed ended linear vector. As used herein, “transduction” refers to the transfer of genetic material into a cell. Transduction efficiency may be measured by techniques well known in the art, for example, a skilled person can measure determine the level of genetic material that has been transferred to a cell by PCR-based assays or westernblotting. In one embodiment, transduction efficiency can be measured against a viral vector containing similar nucleic acid, e.g., promoter, transgene, etc. The viral vector can be any vector known the art, including, AAV, lentiviral vector, adenovirus vector, parvovirus vector, and the like.

All publications, patent applications, patents, patent publications and other references cited herein are incorporated by reference in their entireties for the teachings relevant to the sentence and/or paragraph in which the reference is presented.

The examples, which follow, are set forth to illustrate the present invention, and are not to be construed as limiting thereof.

To the extent that any disclosure in International Application No. PCT/US2019/038515 (PCT Docket Number 046192-092620WOPT), in the name of Asklepios Biopharmaceuticals, Inc. and Richard Jude Samulski filed Jun. 21, 2019 falls within the invention as defined in any one or more of the claims of this application, or within any invention to be defined in amended claims that may in the future be filed in this application or in any patent derived therefrom, and to the extent that the laws of any relevant country or countries to which that or those claims apply provide that the disclosure of International Application No. PCT/US2019/038515 (PCT Docket Number 046192-092620WOPT) is part of the state of the art against that or those claims in or for that or those countries, we hereby reserve the right to disclaim the said disclosure from the claims of the present application or any patent derived therefrom to the extent necessary to prevent invalidation of the present application or any patent derived therefrom.

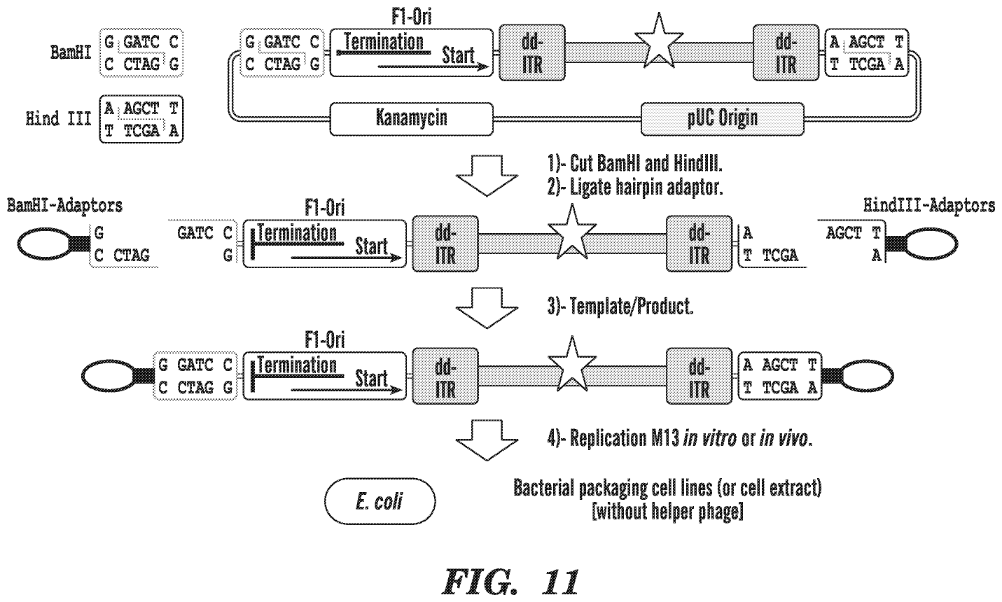

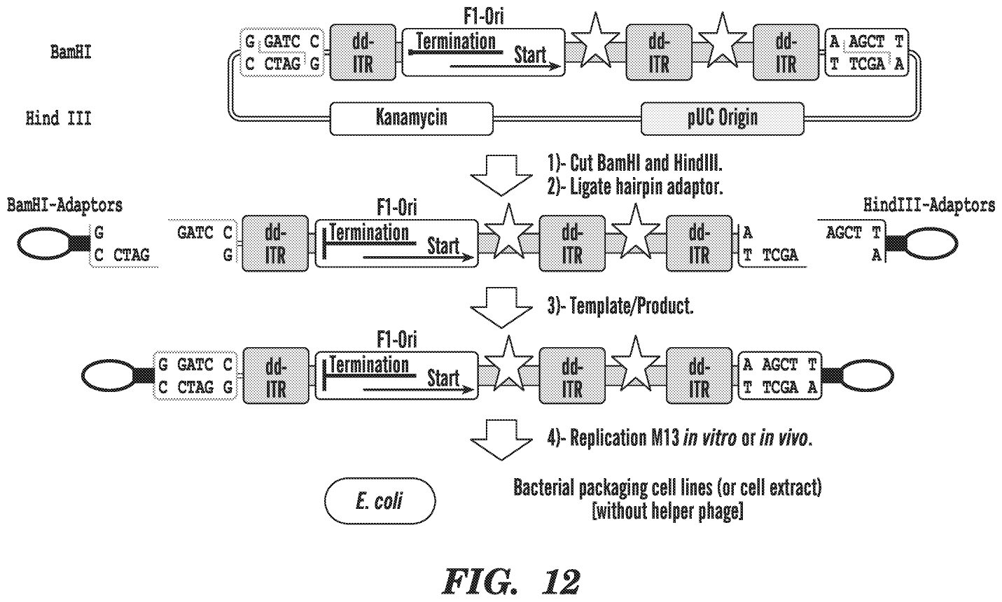

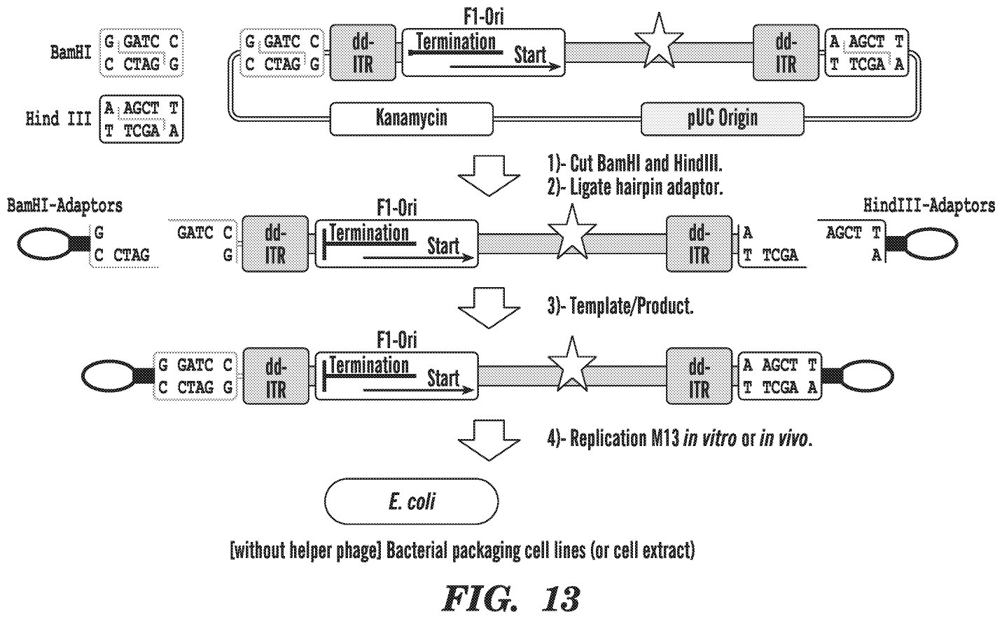

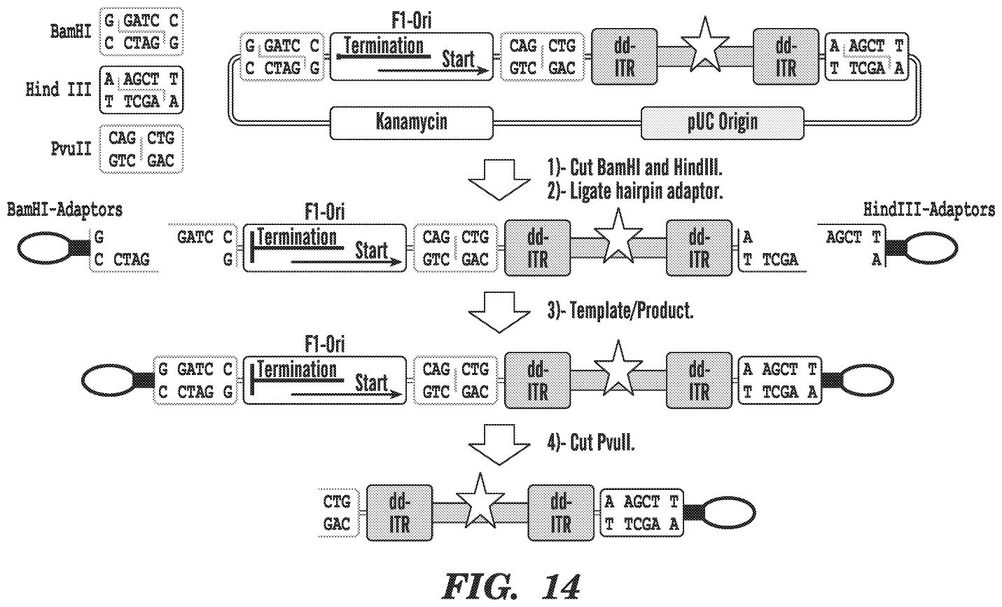

For example, and without limitation, we reserve the right to disclaim any one or more of the following subject-matters from any claim of the present application, now or as amended in the future, or any patent derived therefrom:
A. any subject-matter disclosed in Examples 7 and 8 of International Application No. PCT/US2019/038515 (PCT Docket Number 046192-092620WOPT); or
B. any subject-matter disclosed in FIGS. 11-14 of International Application No. PCT/US2019/038515 (PCT Docket Number 046192-092620WOPT); or
C. a method of manufacturing a circular nucleic acid vector, comprising contacting a host system with a template, wherein the template comprises at least one flanking cleavage sites and: (i) at least one phage origin of replication (ORI); (ii) at least one Terminal Repeat (TR), and; (iii) a promoter sequence operatively linked to a transgene, wherein at least one TR is an AAV Double D-ITR (dd-ITR); (b) incubating the host system for a time sufficient for replication to occur resulting in circular nucleic acid production; and (c) recovering the circular nucleic acid production, wherein the circular nucleic acid self-anneals; or
D. a method of manufacturing a circular nucleic acid vector, comprising contacting a host system with a template, wherein the template comprises at least two flanking cleavage sites and within the sites are: (i) at least one phage origin of replication (ORI); (ii) at least one Terminal Repeat (TR), and; (iii) a promoter sequence operatively linked to a transgene, wherein at least one TR is an AAV Double D-ITR (dd-ITR); (b) incubating the host system for a time sufficient for replication to occur resulting in circular nucleic acid production; and (c) recovering the circular nucleic acid production, wherein the circular nucleic acid self-anneals; or
E. a method of manufacturing circular nucleic acid vectors containing a transgene, the method comprising: (a) transforming a host system with a plasmid template, wherein the plasmid template comprises: (i) a phage origin of replication (ORI); (ii) a truncated phage ORI (e.g., ORIΔ29); (iii) at least one Terminal Repeat (TR), and; (iv) a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand, and wherein at least one TR is an AAV Double D-ITR (dd-ITR);; (b) incubating the host system for a time sufficient for replication to occur resulting in circular nucleic acid production; and (c) recovering the circular nucleic acid produced, wherein the circular nucleic acid self-anneals.
F. any of the circular nucleic acid selected from:
   a. having at least one cleavage site, a phage ORI, at least one dd-ITR, and at least one promotor linked to a transgene in any order from 5′ to 3′; or
   b. having in at least two cleavage sites, a phage ORI, at least one dd-ITR, and at least one promotor linked to a transgene in any order from 5′ to 3′; or
   c. having in at least three cleavage sites, a phage ORI, at least one dd-ITR, and at least one promotor linked to a transgene in any order from 5′ to 3′; or
   d. having at least one cleavage site, at least two phage ORIs, at least one dd-ITR, and at least one promotor linked to a transgene in any order from 5′ to 3′; or
   e. having in at least two cleavage sites, at least two phage ORIs, at least one dd-ITR, and at least one promotor linked to a transgene in any order from 5′ to 3′; or
   f. having in at least three cleavage sites, at least two phage ORIs, at least one dd-ITR, and at least one promotor linked to a transgene in any order from 5′ to 3′; or
   g. having at least one cleavage site, a phage ORI, at least two dd-ITRs, and at least one promotor linked to a transgene in any order from 5′ to 3′; or
   h. having in at least two cleavage sites, a phage ORI, at least two dd-ITRs, and at least one promotor linked to a transgene in any order from 5′ to 3′; or
   i. having in at least three cleavage sites, a phage ORI, at least two dd-ITRs, and at least one promotor linked to a transgene in any order from 5′ to 3′; or
   j. having at least one cleavage site, at least two phage ORIs, at least two dd-ITRs, and at least one promotor linked to a transgene in any order from 5′ to 3′; or
   k. having in at least two cleavage sites, at least two phage ORIs, at least two dd-ITRs, and at least one promotor linked to a transgene in any order from 5′ to 3′; or
l. having in at least three cleavage sites, at least two phage ORIs, at least two dd-ITRs, and at least one promotor linked to a transgene in any order from 5′ to 3′; or
m. having at least one cleavage site, a phage ORI, at least three dd-ITRs, and at least one promotor linked to a transgene in any order from 5′ to 3′; or
n. having in at least two cleavage sites, a phage ORI, at least three dd-ITRs, and at least one promotor linked to a transgene in any order from 5′ to 3′; or
o. having in at least three cleavage sites, a phage ORI, at least three dd-ITRs, and at least one promotor linked to a transgene in any order from 5′ to 3′; or
p. having at least one cleavage site, at least two phage ORIs, at least three dd-ITRs, and at least one promotor linked to a transgene in any order from 5′ to 3′; or
q. having in at least two cleavage sites, at least two phage ORIs, at least three dd-ITRs, and at least one promotor linked to a transgene in any order from 5′ to 3′; or
r. having in at least three cleavage sites, at least two phage ORIs, at least three dd-ITRs, and at least one promotor linked to a transgene in any order from 5′ to 3′; or
s. having at least one cleavage site, a phage ORI, at least four dd-ITRs, and at least one promotor linked to a transgene in any order from 5′ to 3′; or
t. having in at least two cleavage sites, a phage ORI, at least four dd-ITRs, and at least one promotor linked to a transgene in any order from 5′ to 3′; or
u. having in at least three cleavage sites, a phage ORI, at least four dd-ITRs, and at least one promotor linked to a transgene in any order from 5′ to 3′; or
v. having at least one cleavage site, at least two phage ORIs, at least four dd-ITRs, and at least one promotor linked to a transgene in any order from 5′ to 3′; or
   w. having in at least two cleavage sites, at least two phage ORIs, at least four dd-ITRs, and at least one promotor linked to a transgene in any order from 5′ to 3′; or
   x. having in at least three cleavage sites, at least two phage ORIs, at least four dd-ITRs, and at least one promotor linked to a transgene in any order from 5′ to 3′; or
   y. having at least one cleavage site, a phage ORI, at least five dd-ITRs, and at least one promotor linked to a transgene in any order from 5′ to 3′; or
   z. having in at least two cleavage sites, a phage ORI, at least five dd-ITRs, and at least one promotor linked to a transgene, wherein the at least two cleavage sites are flanking the other components in any order from 5′ to 3′; or
   aa. having in at least three cleavage sites, a phage ORI, at least five dd-ITRs, and at least one promotor linked to a transgene in any order from 5′ to 3′; or
   bb. having at least one cleavage site, at least two phage ORIs, at least five dd-ITRs, and at least one promotor linked to a transgene in any order from 5′ to 3′; or
   cc. having in at least two cleavage sites, at least two phage ORIs, at least five dd-ITRs, and at least one promotor linked to a transgene in any order from 5′ to 3′; or
   dd. having in at least three cleavage sites, at least two phage ORIs, at least five dd-ITRs, and at least one promotor linked to a transgene in any order from 5′ to 3′; or
   ee. having at least one cleavage site, a phage ORI, at least six dd-ITRs, and at least one promotor linked to a transgene in any order from 5′ to 3′; or
   ff. having in at least two cleavage sites, a phage ORI, at least six dd-ITRs, and at least one promotor linked to a transgene in any order from 5′ to 3′; or
   gg. having in at least three cleavage sites, a phage ORI, at least six dd-ITRs, and at least one promotor linked to a transgene, wherein the at least two cleavage sites are flanking the other components, and the third cleavage site is downstream of the at least one ORI
   hh. having at least one cleavage site, at least two phage ORIs, at least six dd-ITRs, and at least one promotor linked to a transgene in any order from 5′ to 3′; or
   ii. having in at least two cleavage sites, at least two phage ORIs, at least six dd-ITRs, and at least one promotor linked to a transgene in any order from 5′ to 3′; or
   jj. having in at least three cleavage sites, at least two phage ORIs, at least six dd-ITRs, and at least one promotor linked to a transgene in any order.
G. any of the circular nucleic acids from [E], further comprising at least one additional ITR that is not a dd-ITR.
H. any of the circular nucleic acids from [E], further comprising at least one adaptor sequence.
I. any of the circular nucleic acids from [E], further comprising at least two adaptor sequences.
J. any of the circular nucleic acids from [E], further comprising at least one additional promoter operatively linked to a transgene.

Without limitation, we state that the above reservation of a right of disclaimer applies at least to claims 1-73 as appended to this application and paragraphs 1-75 as set forth at [0150].

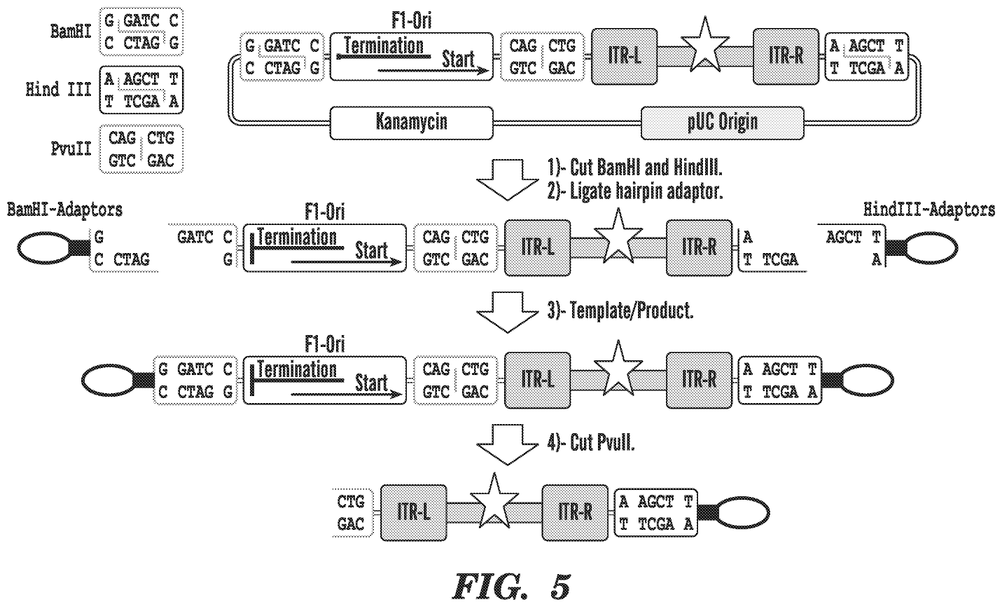

The invention described herein can further be described in the following numbered paragraphs:
1. A method of manufacturing circular nucleic acid vectors containing a transgene, the method comprising:
   a. contacting a host system with a template, wherein the template comprises at least one flanking cleavage sites, and:
      i. at least one phage origin of replication (ORI);
      ii. at least one Terminal Repeat (TR), and;
      iii. a promoter sequence operatively linked to a transgene;
   b. incubating the host system for a time sufficient for replication to occur resulting in circular nucleic acid production; and
   c. recovering the circular nucleic acid production, wherein the circular nucleic acid self-anneals.
2. The method of paragraph 1, wherein the template further comprises a second flanking cleavage sites, and within the two sites are (i)-(iii).
3. The method of any of the proceeding paragraphs, wherein the template further comprises at least one additional cleavage site immediately downstream of the at least one ORI (see e.g., FIG. 5).
4. The method of any of the proceeding paragraphs, further comprising the step of cutting at least one cleavage site of the recovered circular nucleic acid (see e.g., FIG. 5).
5. The method of any of the proceeding paragraphs, further comprising, following recovery, the step of in vitro replication of the circular nucleic acid.
6. The method of any of the proceeding paragraphs, wherein the template further comprises at least one adapter sequence.
7. The method of any of the proceeding paragraphs, wherein the template further comprises at least two adapter sequences.
8. The method of any of the proceeding paragraphs, wherein the adaptor sequence induces closure of cleaved DNA (see e.g., FIGS. 1-5, 7, and 9).
9. The method of any of the proceeding paragraphs, wherein the adaptor sequence further comprises a cleavage site.
10. The method of any of the proceeding paragraphs, wherein the recovered circular nucleic acid is used for delivery of the transgene.
11. The method of any of the proceeding paragraphs, wherein the recovered circular nucleic acid is used for recombinant viral vector production.
12. The method of any of the proceeding paragraphs, wherein the circular nucleic acid is self-annealed and double-stranded.
13. The method of any of the proceeding paragraphs, wherein the vector is single-stranded.
14. The method of any of the proceeding paragraphs, wherein there is a second TR and the promoter sequence operably linked to a transgene is flanked on both sides by a TR.
15. The method of any of the proceeding paragraphs, wherein the ORI is upstream of the left TR.
16. The method of any of the proceeding paragraphs, wherein the ORI is flanked by the TRs and upstream of the promoter sequence operably linked to a transgene.
17. The method of any of the proceeding paragraphs, wherein the host system is a bacterial packaging cell.
18. The method of any of the proceeding paragraphs, wherein the host system is a cell-free system.
19. The method of any of the proceeding paragraphs, wherein the host system is a cell-free system and contains helper phage particles.
20. The method of any of the proceeding paragraphs, wherein the host system is a host cell.
21. The method of any of the proceeding paragraphs, wherein the host cell is a mammalian cell, a bacterial cell, or an insect cell.
22. The method of any of the proceeding paragraphs, wherein the viral vector is an adeno associated virus (AAV), a lentivirus (LV), a herpes simplex virus (HSV), an adeno virus (AV), or a pox virus (PV).
23. The method of any of the proceeding paragraphs, wherein the vector is a DNA or RNA virus.
24. The method of any of the proceeding paragraphs, wherein the virus is an AAV and has a mutant ITR, wherein the mutant ITR is a Double D mutant ITR.
25. The method of any of the proceeding paragraphs, wherein the at least one TR is a mutant ITR, a synthetic ITR, a wild-type ITR, or a non-functional ITR.
26. The method of any of the proceeding paragraphs, wherein the vector has flanking DD-ITRs, and in between the flanking is a promoter operatively linked to a sense strand of the transgene, a replication defective ITR, and an anti-sense complement of the transgene.
27. The method of any of the proceeding paragraphs, wherein the ITR is an AAV ITR
28. The method of any of the proceeding paragraphs, wherein the ORI is located upstream of the ITR, and immediately downstream of the upstream ITR.
29. The method of any of the proceeding paragraphs, wherein the at least one phage ORI is selected from the group consisting of: M13 derived ORI, F1 derived ORI, or Fd derived ORI.
30. The method of any of the proceeding paragraphs, wherein the temple further comprises a second ORI that is a truncated ORI that does not initiate replication.
31. The method of any of the proceeding paragraphs, wherein the truncated ORI is ORIΔ29.
32. The method of any of the proceeding paragraphs, wherein the at least two cleavage sites are a restriction site.
33. The method of any of the proceeding paragraphs, wherein the at least two restriction sites are identical or different.
34. The method of any of the proceeding paragraphs, wherein the restriction site is not found within the transgene sequence.
35. The method of any of the proceeding paragraphs, wherein the cleavage site is cleaved by a nuclease.
36. The method of any of the proceeding paragraphs, wherein the promotor is selected from the group consisting of: a constitutive promoter, a repressible promoter, a ubiquitous promoter, an inducible promoter, a viral promoter, a tissue specific promoter, and a synthetic promoter.
37. The method of any of the proceeding paragraphs, wherein the transgene is a therapeutic gene.
38. A method of manufacturing circular nucleic acid vectors containing a transgene, the method comprising:
   a. transforming a host system with a plasmid template, wherein the plasmid template comprises:
      i. a phage origin of replication (ORI);
      ii. a truncated phage ORI (e.g., ORIΔ29);
      iii. at least one Terminal Repeat (TR), and;
      iv. a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand;
   b. incubating the host system for a time sufficient for replication to occur resulting in circular nucleic acid production; and
   c. recovering the circular nucleic acid production,
   wherein the circular nucleic acid self-anneals.
39. The method of any of the proceeding paragraphs, further comprising a linker and a self-complement linker flanking the ORI.
40. The method of any of the proceeding paragraphs, wherein the transgene contains the sense sequences and the anti-sense complement thereof separated by a linker sequences that will permit the sense and anti-sense strands to bind as a double strand.
41. The method of any of the proceeding paragraphs, wherein the truncated ORI is ORIΔ29.
42. A circular nucleic acid vector manufactured by the methods of any of paragraphs 1-41.
43. A circular nucleic acid vector comprising:
   at least one flanking cleavage sites:
      i. at least one phage origin of replication (ORI);
      ii. at least one Terminal Repeat (TR); and
      iii. a promoter sequence operatively linked to a transgene.
44. The vector of any of the proceeding paragraphs, wherein the template further comprises a second flanking cleavage sites, and within the two sites are (i)-(iii).
45. The vector of any of the proceeding paragraphs, wherein the vector further comprises at least one additional cleavage site immediately downstream of the at least one ORI (see e.g., FIG. 5).
46. The vector of any of the proceeding paragraphs, wherein the vector further comprises at least one adapter sequence.
47. The vector of any of the proceeding paragraphs, wherein the vector further comprises at least two adapter sequences.
48. The vector of any of the proceeding paragraphs, wherein the adaptor sequence induces closure of cleaved DNA (see e.g., FIGS. 1-5, 7, and 9)
49. The vector of any of the proceeding paragraphs, wherein the adaptor sequence further comprises a cleavage site.
50. The vector of any of the proceeding paragraphs, wherein the vector is used for delivery of the transgene.
51. The vector of any of the proceeding paragraphs, wherein the vector is used for recombinant viral vector production.
52. The vector of any of the proceeding paragraphs, wherein the vector is self-annealed and double-stranded.
53. The vector of any of the proceeding paragraphs, wherein the vector is single-stranded.
54. The vector of any of the proceeding paragraphs, wherein there is a second TR and the promoter sequence operably linked to a transgene is flanked on both sides by a TR.
55. The vector of any of the proceeding paragraphs, wherein the ORI is upstream of the left TR.
56. The vector of any of the proceeding paragraphs, wherein the ORI is flanked by the TRs and upstream of the promoter sequence operably linked to a transgene.
57. The vector of any of the proceeding paragraphs, wherein the at least one TR is a mutant ITR, a synthetic ITR, a wild-type ITR, or a non-functional ITR.
58. The vector of any of the proceeding paragraphs, wherein the vector has flanking DD-ITRs, and in between the flanking is a promoter operatively linked to a sense strand of the transgene, a replication defective ITR, and an anti-sense complement of the transgene.
59. The vector of any of the proceeding paragraphs, wherein the ITR is an AAV ITR.
60. The vector of any of the proceeding paragraphs, wherein the ORI is located upstream of the ITR, and immediately downstream of the upstream ITR.
61. The vector of any of the proceeding paragraphs, wherein the phage ORI is selected from the group consisting of: M13 derived ORI, F1 derived ORI, or Fd derived ORI.
62. The vector of any of the proceeding paragraphs, wherein the temple further comprises a second ORI that is a truncated ORI that does not initiate replication. 63. The vector of any of the proceeding paragraphs, wherein the truncated ORI is ORIΔ29.
64. The vector of any of the proceeding paragraphs, wherein the at least two cleavage sites are a restriction site.
65. The vector of any of the proceeding paragraphs, wherein the at least two restriction sites are identical or different.
66. The vector of any of the proceeding paragraphs, wherein the restriction site is not found within the transgene sequence.
67. The vector of any of the proceeding paragraphs, wherein the cleavage site is cleaved by a nuclease.
68. The vector of any of the proceeding paragraphs, wherein the promotor is selected from the group consisting of: a constitutive promoter, a repressible promoter, a ubiquitous promoter, an inducible promoter, a viral promoter, a tissue specific promoter, and a synthetic promoter.
69. The vector of any of the proceeding paragraphs, wherein the transgene is a therapeutic gene.
70. A circular nucleic acid vector comprising:
   i. a phage origin of replication (ORI);
   ii. a truncated phage ORI (e.g., ORIΔ29);
   iii. at least one Terminal Repeat (TR), and;
   iv. a promoter sequence operatively linked to a transgene, wherein the vector comprises, in the 5′ to 3′ direction, the sense sequence and the anti-sense sequence separated by a hairpin sequence that allows for annealing of the sense and anti-sense strand.
71. The vector of any of the proceeding paragraphs, further comprising a linker and a self-complement linker flanking the ORI.
72. The vector of any of the proceeding paragraphs, wherein the transgene contains the sense sequences and the anti-sense complement thereof separated by a linker sequences that will permit the sense and anti-sense strands to bind as a double strand.
73. The vector of any of the proceeding paragraphs, wherein the truncated ORI is ORIΔ29.
74. The vector of any of the proceeding paragraphs, wherein the nuclease is a protelomerase.
75. The vector of any of the proceeding paragraphs, wherein the nuclease is a protelomerase.

### EXAMPLES

### Example 1

### Making Templates that Produce Circular Nucleic Acids

Exemplified herein are methods for making a template useful for generating vectors for expressing the human factor IX minigene in a subject. A plasmid having, from 5′ to 3′, a BAMHI restriction site, a F1 ORI, a PvuII restriction site, an ITR-L, a liver-specific promoter of SEQ ID NO: 247 operatively linked to the coding region of factor IX, ITR-R, and a HINDIII restriction site, is digested with BAMHI and HINDIII restriction enzymes for 24 hours at 37° C. The digest is run on an electrophoresis gel to visualize and isolate the plasmid fragment. The plasmid fragment is cut out of the gel and purified. An adaptor sequence having BAMHI restriction site sequence and an adaptor sequence having a HINDIII restriction site sequence are additionally digested and purified in the same manner.

To form the circular nucleic acid template, adaptor sequences are annealed to the cuts of the plasmid fragment. The purified plasmid fragment and adapter sequences are ligated in the presence of a ligase, such as T4 ligase, and ATP at room temperature for at least 1 hour. The ligation reaction is heat-inactivated at 65° C. for 10 minutes to inactivate the ligase enzyme.

The circular nucleic template acid is transformed into E. coli cells and grown at 37° C., shaking, for 14-16 hours to induce replication of the circular nucleic acid. The circular nucleic acid encoding the factor IX transgene is released from E. coli cells using a bacterial lysis reagent to cause cell lysis. Following release, the factor IX circular nucleic acid is recovered using standard methods, for example, via purification using column chromatography. The self-annealed, circular nucleic acid can be recovered and used directly for delivery of the transgene in vivo, or used for viral production (see Example 2).

The recovered factor IX circular nucleic acid is further digested with a PvuII restriction enzyme for 24 hours at 25° C. to cut the PvuII cleavage site (see e.g., FIG. 5). Cutting PvuII removes the ORI and creates an open-end on the factor IX nucleic acid construct. The open ended circular nucleic acid can be used for in vivo delivery of transgene or for production of recombinant viral DNA. The circular nucleic acid need not be digested with PvuII to be used for recombinant viral production, or for in vivo delivery of transgene.

### Example 2

### Circular Nucleic Acids Persist in Receipt Cells

In order to demonstrate clinical relevance, an expression cassette containing the human factor IX minigene driven by a liver-specific promoter (SEQ ID NO: 247) is delivered to the liver of hemophilia B mice using different vectors (the self-annealed, circular nucleic acid of the instant invention, i.e., produced in Example 1), corresponding linear DNA, and corresponding plasmid DNA. The mice are analyzed for vector presence and for gene expression of factor IX by analysis of serum human factor IX concentrations at various times following the injections (3, 4, 5, 6, 7, 8, 9, 10 weeks, and at 3 months, 5 months, 10 months, over a year, etc).

The recipients of the circular nucleic acid containing the factor IX expression cassette are expected to have higher amounts of vector present over time, and have higher concentrations of the factor IX than recipients of ss linear DNA and plasmid DNA controls. The amount of vector present and the concentrations of the human factor IX is expected to persist over time in the recipients of the circular nucleic acid, and to decrease over time in the recipients of the controls. In addition, over time, the expression of the factor IX in the circular nucleic acid in recipient mice is expected to persist at substantially higher levels and over a substantially longer period of time than expression of the Factor IX in recipients of the controls. This is determined by quantitation of human factor IX in serum by ELISA analysis, and identification of correction of their bleeding diathesis, and also by quantitation of vector in the livers of treated mice by Southern blot analysis at different times following vector administration. In addition, the number of hepatic nuclei containing the vectors in the treated mice are determined by in situ hybridization of liver sections, to verify that the relative number of hepatic nucleic containing the vectors is similar.

Molecular Structure of Delivered DNA In Vivo

The vector is expected to form concatemers in recipient tissue cells that persist over time. The concatemers persist extrachromasomally, or integrate into the host cell genome. To demonstrate this, recipient mice liver tissue is analyzed by Southern blot analysis for the molecular structure of the vector DNA. The DNA is isolated and then digested with a restriction endonuclease that either does not cut within the vector, or cuts once in the expression cassette. Analysis of the cut DNA is performed to determine integration of DNA into the mouse genome or retention extrachromasomally. The production of a high molecular weight band in all samples from digestion with an endonuclease that fails to cut within the vector DNA, is consistent with either the integration of the DNA vector into the mouse genome or rapid formation of concatemers in vivo. These two possibilities are distinguished by digestion of the liver DNA samples with the restriction endonuclease that cuts once through the expression cassette. Conversion of the high molecular DNA signal into a DNA ladder by this digestion would indicate concatemerization in vivo.

The majority of the high-molecular-weight DNA may originate from concatemers. However, to determine if this is extrachromasomal or integrated into the genome, the mice are injected with either the circular nucleic acid of the invention, or an integrating factor IX transposon (Yant et al., Nat. Genet. 25: 35-41) to a ⅔ partial hepatectomy. In the transposon group, the transposase, expressed from one plasmid, mediates the release of the human factor IX expression cassette flanked by the transposon ITR from a second plasmid and the insertion of the released transgene expression cassette into the mouse genome. If the partial hepatectomy results in one or two rounds of hepatocyte cell division and a significant loss of extrachromosomal DNA, this would indicate that in contrast to integrating plasmid-infused mice, whose transgene expression should be unchanged by the induction of hepatocyte proliferation, the vector DNA treated mice will show a 10-fold drop in gene expression after partial hepatectomy over the same time period. These data would indicate that transcriptionally active circular nucleic acids of the invention remain predominantly extrachromosomal in the liver. The alternative results would indicate that the active circular nucleic acids are integrated into the liver cell chromosomes.

Methods

Animal studies. Eight- to ten-week-old female C57BL/6 mice are obtained from Taconic Farms, Inc. (Germantown, NY). All animal procedures are performed under the guidelines set forth by Stanford University and the National Institute of Health. Forty micrograms of DNA in 2 ml 0.85% saline is injected into mouse tail veins as previously described (Liu et al., (1999) Gene Ther. 6: 1258-1266; Zhang et al., (1999) Human Gene Therapy 10: 1735-1737). For each injection, the mass of DNA is the same while the molar ratio can vary (e.g., by twofold). In other studies, small variations in molar ratios is not expected to significantly affect gene expression. Mice are bled periodically by a retro-orbital technique. In some cases, mice are subjected to a surgical 2/3 partial hepatectomy as previously described (Park et al., Nat. Genet. 24: 49-52 (2000)). The bleeding times in mice are determined by measuring the time required for clotting of the blood from a 2- to 3-mm tail snip, as previously described (Yant et al., (2000) Nat. Genet. 25: 35-41).

In situ hybridization. Five-micrometer sections of paraffin-embedded livers of the mice 2 to 3 weeks after receiving 40 μg of the respective construct DNA via tail vein infusion are processed for in situ hybridization according to the protocol described previously (Miao et al. (2000) J. Virol. 74: 3793-3803). Following deparaffinization, rehydration, denaturation, and digestion with proteinase, the sections are incubated with a denatured DNA probe specific for the vector labeled with digoxigenin using the DIG labeling kit from Roche Molecular Biochemicals (Indianapolis, IN). After hybridization, the sections are incubated with a goat anti-digoxigenin antibody conjugated with alkaline phosphatase, and the alkaline phosphatase-bound vector DNA is visualized by nitroblue tetrazolium chloride-5-4-chloro-3-indolyl phosphate (Roche Molecular Biochemicals).

ELISA quantitation. After DNA delivery, mouse blood is collected periodically, and human factor IX (hFIX) (Walter et al., (1996) PNAS USA 93: 3056-3061) is quantitated by ELISA.

Southern blot analyses. Mice are sacrificed at period times after DNA injection and total liver DNA is prepared by a salting-out procedure. Twenty micrograms of liver DNA is digested with restriction enzymes, separated by gel electrophoresis, and analyzed by Southern blot hybridization using a cDNA as a probe. Radioactive DNA bands are quantitated by phosphoimager analyses.

The present invention is not to be limited in scope by the exemplified embodiments disclosed herein which are intended as illustrations of single aspects of the invention, and any clones, DNA or amino acid sequences which are functionally equivalent are within the scope of the invention. Indeed, various modifications of the invention, in addition to those shown and described herein, will become apparent to those skilled in the art from the foregoing description and accompanying drawings. Such modifications are intended to fall within the scope of the appended claims.

### Example 3

### Circular Nucleic Acids Have Enhanced Transduction

The self-annealed, circular nucleic acids expressing factor IX produced in Example 1 exhibit enhanced transduction as compared to a standard vector expressing a nucleic acid construct, e.g., a lentivirus vector. Each construct was injected into a mouse via tail-vein injection. Expression of the factor IX transgene is driven by a liver-specific promoter, thus expression is expected to be restricted to the liver. 7 days post injection (dpi), mice are sacrificed and livers are obtained and probed for protein expression of the factor IX transgene via westernblotting. Transgene expression from the circular nucleic acids is found to be enhanced relative to the standard vector by 70-fold. All results are confirmed in multiple experiments using independent preparations of vectors purified by several methods (e.g., density ultracentrifugation, affinity chromatography) to ensure that measured effects are not specific to batch or purification method. Transduction of the circular nucleic acids expressing factor IX is further found to be significantly enhanced as compared to the transduction of an AAV vector expressing a similar nucleic acid construct. These experiments are repeated with a circular nucleic acid expressing human Acid-a1,4-Glucosidase (GAA), and enhanced transduction is similarly observed as compared to a standard vector.

To further ensure an appropriate time point was examined, a time course of transgene expression from circular nucleic acids and standard nucleic acid vector is performed, with measurements collected at 7, 14, 21 and 42 dpi. Expression kinetics appear the same, with robust expression observed at 7 dpi, increasing by 7.5-fold between days 7 and 42 following injection of the standard vector and by 12-fold during same time period following injection of the circular nucleic acids. At all time points, circular nucleic acids outperform the standard vector by approximately two orders of magnitude (100-fold at day 7, 176-fold at day 14, 81-fold at day 21, and 159-fold at day 42). Taken together, these data demonstrate that the circular nucleic acids of the invention are highly effective vectors to enhance transgene transduction.

### Example 4

### Manufacturing of Viral Vectors Using Circular Nucleic Acids

The open- and closed-ended factor IX nucleic acid constructs produced in Example 1 are used to manufacture viral vectors in a stable cell line for AAV production, Pro 10 cells. These stable Pro 10 cells for AAV production, e.g., as described in U.S. Pat. No. 9,441,206, are ideal for scalable production of AAV vectors. The cell line is contacted with the factor IX nucleic acid constructs via transfection to express the circular nucleic acid. Expression of factor IX nucleic acid constructs is confirmed via PCR-based assays using primers specific for the plasmid.

Transfection. Stable Pro10 cells are transfected with factor IX nucleic acid constructs and are also transfected with a Packaging plasmid encoding Rep2 and serotype-specific Cap2: alternatively, AAV-Rep/Cap is also provided as self-annealed circular nucleic acids made by the methods described herein, and/or the Ad-Helper plasmid (XX680: encoding adenoviral helper sequences) is also provided as self-annealed circular nucleic acids made by the methods described herein.

On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to transfection. Two days post-seeding the wave bag, cell culture counts are taken and the cell culture is then expanded/diluted before so transfection. The wave bioreactor cell culture is then transfected. Cell culture are harvested from the wave bio-reactor bag at least 48 hours post-transfection.

Titer: AAV titers are calculated after DNase digestion using qPCR against a standard curve (AAV ITR specific) and primers specific to the factor IX nucleic acid construct.

Harvesting Suspension Cells from Shaker Flasks and 60 Wave Bioreactor Bags. 48 hours post-transfection, cell cultures are collected into 500 mL polypropylene conical tubes (Corning) either by pouring from shaker flasks or pumping from wave bioreactor bags. The cell cultures are then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 mM. At this point, the pellet could either be stored in NLT-60° C. or continued through purification.

Titering rAAV from Cell Lysate Using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl2, 5 mM MgCl2, 50 mM Tris-HCl pH 8.0) followed by sonication to lyse the cells efficiently. 300 uL is then removed and placed into a 1.5 mL microfuge tube. 140 units of DNase I is then added to each sample and incubated at 37° C. for 1 hour. To determine the effectiveness of the DNase digestion, 4-5 mg of the factor IX nucleic acid construct is spiked into a non-transfected cell lysate with and without the addition of DNase. 504 of EDTA/Sarkosyl solution (6.3% sarkosyl, 62.5 mM EDTA pH 8.0) is added to each tube and incubated at 70° C. for 20 minutes. 50 μL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers 2s designed to bind to a homologous sequence on the backbones of plasmids XX680, pXR2 and factor IX nucleic acid constructs. The second qPCR reaction is set up using a set of primers to bind and amplify a region within the factor IX mini gene. qPCR is conducted using Sybr green reagents and Light cycler 480 from 30 Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc), which are incorporated herein by reference in their entireties.

### Example 5

### Concatameric DNA Amplification

Circular nucleic acids containing a nucleic acid encoding the human factor IX gene, a DD-ITR, and telomeric ends comprising the binding sequence of a protelomerase TelN is used as the DNA template. A single palindromic oligonucleotide complementary to a section of one half of the palindromic sequence that comprises the telomeric ends of the template is used as a specific primer. The primer binds to two identical sites on the DNA template.

Denaturation of the circular nucleic acid and the annealing of the single primer is carried out in an annealing/denaturation buffer containing, for example, 30 mM Tris-HCl pH 7.5, 20 mM KCl, 2.5 mM MgCl2. Denaturation is carried out by heating to 95° C. for 1 min and maintaining at this temperature for 1 to 10 minutes followed by a carefully controlled cooling profile optimized for the maximum binding of the specific primer to the template. The temperature is then reduced to the optimum for DNA amplification by a suitable DNA polymerase. One such suitable enzyme is phi29 isolated from the Bacillus subtilis phage phi29 that works optimally at 30° C.

A suitable volume of reaction buffer containing the enzymes phi29 and PPi (Yeast Inorganic pyrophosphatase) is then added to the annealed DNA/primer reaction. The reaction mixture is incubated at around 30° C. for between 5 and 20 hours or longer. A suitable reaction buffer typically contains 35 mM Tris-HCl, 50 mM KCl, 2.5 mM MgCl2, 10 mM (NH4)2SO4, 4 mM DTT, 1 mM dNTP.

Concatameric DNA amplified by RCA is then incubated at 30° C. with the protelomerase TelN in a suitable buffer such as 10 mM Tris HCl pH 7.6, 5 mM CaCl2, 50 mM potassium glutamate, 0.1 mM EDTA, 1 mM DTT until the reaction is complete. The resulting closed linear DNA product may be purified, for example, by gel electrophoresis or a suitable chromatographic method depending on the amount to be purified.

These methods provide for a cyclic reaction wherein the product is identical to the template. This reaction is easily scaled up from a very small amount of template by carrying out additional cycles of the methods steps.

1. A method of manufacturing circular nucleic acid vectors containing a transgene, the method comprising:
a. contacting a host system with a template, wherein the template comprises at least one flanking cleavage sites and:
i. at least one phage origin of replication (ORI);
ii. at least one Terminal Repeat (TR), and;
iii. a promoter sequence operatively linked to a transgene;
b. incubating the host system for a time sufficient for replication to occur resulting in circular nucleic acid production; and
c. recovering the circular nucleic acid produced in b.,
wherein the circular nucleic acid self-anneals.

2. The method of claim 1, wherein the template further comprises a second flanking cleavage sites, and within the two sites are (i)-(iii).

3. The method of claim 1 or 2, wherein the template further comprises at least one additional cleavage site immediately downstream of the at least one ORI (see e.g., FIG. 5).

4. The method of any of claims 1-3, further comprising the step of cutting at least one cleavage site of the recovered circular nucleic acid (see e.g., FIG. 5).

5. The method of any of claims 1-4, further comprising, following recovery, the step of in vitro replication of the circular nucleic acid.

6. The method of any one of claims 1-5, wherein the template further comprises at least one adapter sequence.

7. The method of any one of claims 1-6, wherein the template further comprises at least two adapter sequences.

8. The method of claim 6 or 7, wherein the adaptor sequence induces closure of cleaved DNA (see e.g., FIGS. 1-5, 7, and 9).

9. The method of claim 6 or 7, wherein the adaptor sequence further comprises a cleavage site.

10. The method of any of claims 1-9, wherein the recovered circular nucleic acid is used for delivery of the transgene.

11. The method of any of claims 1-9, wherein the recovered circular nucleic acid is used for recombinant viral vector production.

12. The method of any one of claims 1-11, wherein the circular nucleic acid is self-annealed and double-stranded.

13. The method of any one of claims 1-12, wherein the vector is single-stranded.

14. The method of any one of claims 1-13, wherein there is a second TR and the promoter sequence operably linked to a transgene is flanked on both sides by a TR.

15. The method of any one of claims 1-14, wherein the ORI is upstream of the left TR.

16. The method of any one of claims 1-15, wherein the ORI is flanked by the TRs and upstream of the promoter sequence operably linked to a transgene.

17. The method of any one of claims 1-16, wherein the host system is a bacterial packaging cell.

18. The method of any one of claims 1-17, wherein the host system is a cell-free system.

19. The method of any one of claims 1-18, wherein the host system is a cell-free system and contains helper phage particles.

20. The method of any one of claims 1-19, wherein the host system is a host cell.

21. The method of claim 20, wherein the host cell is a mammalian cell, a bacterial cell, or an insect cell.

22. The method of claim 11, wherein the viral vector is an adeno associated virus (AAV), a lentivirus (LV), a herpes simplex virus (HSV), an adeno virus (AV), or a pox virus (PV).

23. The method of claims 11 and 22, wherein the vector is a DNA or RNA virus.

24. The method of claim 22, wherein the virus is an AAV and has a mutant ITR, wherein the mutant ITR is a Double D mutant ITR.

25. The method of any one of claims 1-24, wherein the at least one TR is a mutant ITR, a synthetic ITR, a wild-type ITR, or a non-functional ITR.

26. The method of any one of claims 1-25, wherein the vector has flanking DD-ITRs, and in between the flanking DD-ITRs is a promoter operatively linked to a sense strand of the transgene, a replication defective ITR, and an anti-sense complement of the transgene.

27. The method of any one of claims 25-26, wherein the ITR is an AAV ITR

28. The method of any one of claims 1-27, wherein the ORI is located upstream of the ITR, and immediately downstream of the upstream ITR.

29. The method of any one of claims 1-28, wherein the at least one phage ORI is selected from the group consisting of: M13 derived ORI, F1 derived ORI, and Fd derived ORI.

30. The method of any one of claims 1-29, wherein the temple further comprises a second ORI that is a truncated ORI that does not initiate replication.

31. The method of claim 30, wherein the truncated ORI is ORIΔ29.

32. The method of any one of claims 1-31, wherein the at least two cleavage sites are a restriction site.

33. The method of claim 32, wherein the at least two restriction sites are identical or different.

34. The method of claim 32, wherein the restriction site is not found within the transgene sequence.

35. The method of any one of claims 1-34, wherein the cleavage site is cleaved by a nuclease.

36. The method of any one of claims 1-35, wherein the promotor is selected from the group consisting of: a constitutive promoter, a repressible promoter, a ubiquitous promoter, an inducible promoter, a viral promoter, a tissue specific promoter, and a synthetic promoter.

37. The method of any one of claims 1-36, wherein the transgene is a therapeutic gene.

38. A method of manufacturing circular nucleic acid vectors containing a transgene, the method comprising:
a. transforming a host system with a plasmid template, wherein the plasmid template comprises:
i. a phage origin of replication (ORI);
ii. a truncated phage ORI (e.g., ORIΔ29);
iii. at least one Terminal Repeat (TR), and;
iv. a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand;
b. incubating the host system for a time sufficient for replication to occur resulting in circular nucleic acid production; and
c. recovering the circular nucleic acid produced,
wherein the circular nucleic acid self-anneals.

39. The method of claim 38, further comprising a linker and a self-complement linker flanking the ORT.

40. The method of claim 38 or 39, wherein the transgene contains the sense sequences and the anti-sense complement thereof separated by a linker sequence that will permit the sense and anti-sense strands to bind as a double strand.

41. The method of any one of claims 38-40, wherein the truncated ORI is ORIΔ29.

42. A circular nucleic acid vector manufactured by the methods of any one of claims 1-41.

43. A circular nucleic acid vector comprising:
at least one flanking cleavage sites, and:
i. at least one phage origin of replication (ORI);
ii. at least one Terminal Repeat (TR); and
iii. a promoter sequence operatively linked to a transgene.

44. The vector of claim 43, wherein the template further comprises a second flanking cleavage sites, and within the two sites are (i)-(iii).

45. The vector of claim 43 or 44, wherein the vector further comprises at least one additional cleavage site immediately downstream of the at least one ORI (see e.g., FIG. 5).

46. The vector of any one of claims 43-45, wherein the vector further comprises at least one adapter sequence.

47. The vector of any one of claims 43-46, wherein the vector further comprises at least two adapter sequences.

48. The vector of claim 46 or 47, wherein the adaptor sequence induces closure of cleaved DNA (see e.g., FIGS. 1-5, 7, and 9)

49. The vector of claim 46 or 47, wherein the adaptor sequence further comprises a cleavage site.

50. The vector of any of claims 43-49, wherein the vector is used for delivery of the transgene.

51. The vector of any of claims 43-49, wherein the vector is used for recombinant viral vector production.

52. The vector of any one of claims 43-51, wherein the vector is self-annealed and double-stranded.

53. The vector of any one of claims 43-52, wherein the vector is single-stranded.

54. The vector of any one of claims 43-53, wherein there is a second TR and the promoter sequence operably linked to a transgene is flanked on both sides by a TR.

55. The vector of any one of claims 43-54, wherein the ORI is upstream of the left TR.

56. The vector of any one of claims 43-55, wherein the ORI is flanked by the TRs and upstream of the promoter sequence operably linked to a transgene.

57. The vector of any one of claims 43-56, wherein the at least one TR is a mutant ITR, a synthetic ITR, a wild-type ITR, or a non-functional ITR.

58. The vector of any one of claims 43-57, wherein the vector has flanking DD-ITRs, and in between the flanking DD-ITRs is a promoter operatively linked to a sense strand of the transgene, a replication defective ITR, and an anti-sense complement of the transgene.

59. The vector of claim 57 or 58, wherein the ITR is an AAV ITR.

60. The vector of any one of claims 43-59, wherein the ORI is located upstream of the ITR, and immediately downstream of the upstream ITR.

61. The vector of any one of claims 43-60, wherein the phage ORI is selected from the group consisting of: M13 derived ORI, F1 derived ORI, and Fd derived ORI.

62. The vector of any one of claims 43-61, wherein the temple further comprises a second ORI that is a truncated ORI that does not initiate replication.

63. The vector of any one of claims 43-62, wherein the truncated ORI is ORIΔ29.

64. The vector of any one of claims 43-63, wherein the at least two cleavage sites are a restriction site.

65. The vector of claim 64, wherein the at least two restriction sites are identical or different.

66. The vector of claim 64, wherein the restriction site is not found within the transgene sequence.

67. The vector of any one of claims 43-66, wherein the cleavage site is cleaved by a nuclease.

68. The vector of any one of claims 43-67, wherein the promotor is selected from the group consisting of: a constitutive promoter, a repressible promoter, a ubiquitous promoter, an inducible promoter, a viral promoter, a tissue specific promoter, and a synthetic promoter.

69. The vector of any one of claims 43-68, wherein the transgene is a therapeutic gene.

70. A circular nucleic acid vector comprising:
i. a phage origin of replication (ORI);
ii. a truncated phage ORI (e.g., ORIΔ29);
iii. at least one Terminal Repeat (TR), and;
iv. a promoter sequence operatively linked to a transgene, wherein the vector comprises, in the 5′ to 3′ direction, the sense sequence and the anti-sense sequence separated by a hairpin sequence that allows for annealing of the sense and anti-sense strand.

71. The vector of claim 70, further comprising a linker and a self-complement linker flanking the ORT.

72. The vector of claim 70 or 71, wherein the transgene contains the sense sequences and the anti-sense complement thereof separated by a linker sequence that will permit the sense and anti-sense strands to bind as a double strand.

73. The vector of any one of claims 70-72, wherein the truncated ORI is ORIΔ29.

74. A method of delivering a transgene, the method comprising administering any of the circular nucleic acids of any of claims 42-73.

75. The method of claim 74, wherein administering is in vitro, in vivo, or ex vivo.

76. Use of any of the circular nucleic acids of any of claims 42-73 for delivering a transgene.
